(19) United States
(12) Patent Application Publication
(10) Pub. No.: US 2025/0283115 A1
(43) Pub. Date: Sep. 11, 2025

(54) ENGINEERED HIGH FIDELITY OMNI-50 NUCLEASE VARIANTS

(71) Applicant: EmendoBio Inc., Wilmington, DE (US)

(72) Inventors: Lior IZHAR, Tel Aviv (IL); Rafi EMMANUEL, Ramla (IL); Liat ROCKAH, Rishon Lezion (IL); Asael HERMAN, Ness-ziona (IL)

(73) Assignee: EmendoBio Inc., Wilmington, DE (US)

(21) Appl. No.: 18/682,982

(22) PCT Filed: Aug. 12, 2022
(86) PCT No.: PCT/US2022/074930

§ 371 Date: Feb. 12, 2024

Related U.S. Application Data
(60) Provisional application No. 63/232,571, filed on Aug. 12, 2021.
(60) Provisional application No. 63/332,214, filed on Apr. 18, 2022.
(60) Provisional application No. 63/333,037, filed on Apr. 20, 2022.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

The present invention is directed to, inter alia, composition and methods for genome editing. Specifically, a non-naturally occurring OMNI-50 nuclease variant having a wild-type OMNI-50 protein sequence (SEQ ID NO: 1) comprising an amino acid substitution in at least one of the following positions: R61, Y437, R478, A493, Y545, G606, K688, L690, E695, L718, R788, Q803, L805, L844, V981, K965, and K1036.

This application is a U.S. National Stage of PCT International Application No. PCT/US2022/074930 filed Aug. 12, 2022, which claims priority to of U.S. Provisional Application Nos. 63/333,037 filed Apr. 20, 2022, 63/332,214 filed Apr. 18, 2022, and 63/232,571 filed Aug. 12, 2021, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “ENGOMNI50_5798-0046US01.xml”, which is 290,097 bytes in size, and which was created on May 12, 2025 in the IBM-PC machine format, having an operating system compatibility with MS-Windows, which is contained in the XML file filed May 12, 2025 as part of this application.

### BACKGROUND OF INVENTION

Targeted genome modification is a powerful tool that can be used to reverse the effect of pathogenic genetic variations and therefore has the potential to provide new therapies for human genetic diseases. Current genome engineering tools, including engineered zinc finger nucleases (ZFNs), transcription activator-like effector nucleases (TALENs), and most recently, RNA-guided DNA endonucleases such as CRISPR/Cas, produce sequence-specific DNA breaks in a genome. The modification of the genomic sequence occurs at the next step and is the product of the activity of a cellular DNA repair mechanism triggered in response to the newly formed DNA break. These mechanisms may include, for example: (1) classical non-homologous end-joining (NHEJ) in which the two ends of the break are ligated together in a fast but also inaccurate manner (i.e. frequently resulting in mutation of the DNA at the cleavage site in the form of small insertion or deletions) or (2) homology-directed repair (HDR) in which an intact homologous DNA donor is used to replace the DNA surrounding the cleavage site in an accurate manner. Minimal off-target activity of the initial DNA damage inducer is required for efficient and safe genome editing.

### SUMMARY OF THE INVENTION

Disclosed herein are engineered Clustered Regularly Interspaced Short Palindromic Repeats (CRISPRs)/CRISPR-associated OMNI-50 nucleases with altered and improved target specificity and their use in genomic engineering, epigenomic engineering, genome targeting, genome editing, and in vitro diagnostics.

In some embodiments, there is provided a variant of an OMNI-50 nuclease with increased specificity as compared to the wild-type OMNI-50 nuclease, as well as methods of using the improved variants. Advantageously, when the engineered variant OMNI-50 nucleases are active in a CRISPR endonuclease system, the CRISPR endonuclease system displays reduced off-target editing activity and maintained on-target editing activity relative to a wild-type CRISPR endonuclease system in which a wild-type OMNI-50 nuclease is active. For example, an engineered variant OMNI-50 nuclease may display improved allele-specific discrimination, e.g. specific binding and activity at a target region which contains a heterozygous SNP present in only the targeted allele and not present in the non-targeted allele.

In some embodiments, there is provided a variant of an OMNI-50 nickase with increased specificity as compared to the wild-type OMNI-50 nickase. In some embodiments, there is provided a variant of an OMNI-50 dead nuclease with increased specificity as compared to the wild-type OMNI-50 dead nuclease. For example, the catalytic site of any one of the OMNI-50 nuclease variants provided herein may be modified such that the variant has nickase activity, such that it is capable of performing single-strand DN A cuts. Alternatively, the catalytic site of any one of the OMNI-50 nuclease variants provided herein may be modified such that the variant has no nuclease activity, i.e. a dead nuclease.

According to some embodiments of the present invention, there is provided a variant of OMNI-50 nuclease protein comprising a sequence that is at least 80% identical to the amino acid sequence of wild-type OMNI-50 nuclease protein (SEQ ID NO: 1).

According to some embodiments of the present invention, there is provided a non-naturally occurring OMNI-50 nuclease variant having a wild-type OMNI-50 protein sequence (SEQ ID NO: 1) comprising an amino acid substitution in at least one of the following positions: R61, Y437, R478, A493, Y545, G606, K688, L690, E695, L718, R788, Q803, L805, L844, K965, V981, and K1036.

According to some embodiments of the present invention, there is provided a CRISPR system comprising any one of the OMNI-50 nuclease variant disclosed herein complexed with a guide RNA molecule that targets a DNA target site, wherein the CRISPR system displays reduced off-target editing activity relative to a wild-type CRISPR system comprising a wild-type OMNI-50 nuclease protein and the guide RNA molecule.

In some embodiments, the OMNI-50 variant nuclease exhibits increased specificity to a target site when complexed with a guide RNA targeting the OMNI-50 variant to the target site compared to a wild-type OMNI-50 nuclease (SEQ ID NO: 1).

In some embodiments, the OMNI-50 nuclease variant is a nickase having an inactivated RuvC domain created by an amino acid substitution at a position provided for the CRISPR nuclease in column 1 of the table below. In some embodiments, the nickase further comprises an amino acid substitution in at least one of the following positions: R61, Y437, R478, A493, Y545, G606, K688, L690, E695, L718, R788, Q803, L805, L844, K965, V981, and K1036.

In some embodiments, the OMNI-50 nuclease variant is a nickase having an inactivated HNH domain created by an amino acid substitution at a position provided for the CRISPR nuclease in column 2 of the table below. In some embodiments, the nickase further comprises an amino acid substitution in at least one of the following positions: R61, Y437, R478, A493, Y545, G606, K688, L690, E695, L718, R788, Q803, L805, L844, K965, V981, and K1036.

In some embodiments, the OMNI-50 nuclease variant is a catalytically dead nuclease having an inactivated RuvC domain and an inactivated HNH domain created by substitutions at the positions provided for the CRISPR nuclease in column 3 of the table below. In some embodiments, the catalytically dead nuclease comprises an amino acid substitution in at least one of the following positions: R61, Y437, R478, A493, Y545, G606, K688, L690, E695, L718, R788, Q803, L805, L844, K965, V981, and K1036.

TABLE
Positions affecting OMNI-50 nuclease activity

| Nickase having | Nickase having | Dead nuclease having |
|---|---|---|
| inactivated | inactivated | inactivated RuvC and |
| RuvC domain | HNH domain | HNH domains |
| D12 or E785 or | D875* or H876 | (D12 or E785 or H1008 |
| H1008 or D1011 | or N899 | or D1011) and (D875* |
| | | or H876 or N899) |

The above table lists alternative positions to be substituted to generate a nickase having an inactivated RUVC domain, alternative positions to be substituted to generate a nickase having an inactivated HNH domain, and alternative positions to be substituted to generate a catalytically dead nuclease having inactivated RUVC and HNH domains. Substitution to any other amino acid is permissible for each of the amino acid positions indicated in columns 1-3, except if followed by an asterisk, which indicates that any substitution other than aspartic acid (D) to glutamic acid (E) or glutamic acid (E) to aspartic acid (D) results in inactivation.

Throughout the text OMNI-50 nuclease variants are referred to, however, any of these variants may be modified to have nickase activity (i.e. nucleases which create a single-strand DNA break as opposed to a double-strand break) or to have no nuclease activity (i.e. a catalytically dead nuclease).

Accordingly, point mutations can be introduced into any one of the variants described herein to modify or abolish their nuclease activity while still retaining their ability to specifically bind DNA in a sgRNA-programmed manner. Any one of these variants can specifically target a desired DNA target sequence via a guide RNA molecule. The variant-guide complex will also carry any molecule attached to the complex to the target site. Thus, this disclosure also contemplates fusion proteins comprising such variants and a DNA modifying domain (e.g., a deaminase, a nuclease, a nickase, a recombinase, a methyltransferase, a methylase, an acetylase, an acetyltransferase, a transcriptional activator, or a transcriptional repressor domain), as well as the use of such fusion proteins in correcting mutations in a genome (e.g., the genome of a human subject) that are associated with disease, or generating mutations in a genome (e.g., the human genome) to decrease or prevent expression of a gene.

In some embodiments, any of the variants provided herein may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Thus, any one of the OMNI-50 nuclease, nickase, or dead-nuclease variants may be fused (e.g. directly fused or fused via a linker) to another DNA modulating or DNA modifying enzyme, including, but not limited to, base editors such as a deaminase, a reverse transcriptase (e.g. for use in prime editing, see Anzaolone et al. (2019)), an enzyme that modifies the methylation state of DNA (e.g. a methyltransferase), or a modifier of histones (e.g. a histone acetyl transferase). Indeed, the OMNI-50 nuclease, nickase, inactive variants described herein may be fused to a DNA modifying enzyme or an effector domain thereof. Examples of DNA modifiers include but are not limited to: a deaminase, a nuclease, a nickase, a recombinase, a methyltransferase, a methylase, an acetylase, an acetyltransferase, a reverse transcriptase, an helicase, an integrase, a ligase, a transposase, a demethylase, a phosphatase, a transcriptional activator, or a transcriptional repressor. In some embodiments, any of the OMNI-50 variants provided herein are fused to a protein that has an enzymatic activity. In some embodiments, the enzymatic activity modifies a target DNA molecule. the OMNI-50 variants described herein or fusion proteins thereof, may be used to correct or generate one or more mutations in a gene associated with disease, or to increase, correct, decrease or prevent expression of a gene.

According to some embodiments of the present invention, there is provided a method for gene editing having reduced off-target editing activity, comprising contacting a DNA target site with an active CRISPR system comprising any one of the OMNI-50 nuclease variant proteins described herein.

According to some embodiments, there is provided a method for gene editing having reduced off-target editing activity and/or increased on-target editing activity, comprising: contacting a target site locus with an active CRISPR system comprising a variant OMNI-50 nuclease protein of any one of the variants described herein, wherein the active CRISPR system displays reduced off-target editing activity and maintained on-target editing activity relative to a wild-type CRISPR system having a wild-type OMNI-50 nuclease protein.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: A schematic showing the nuclease optimization platform is shown. Libraries of nuclease variants are created using a combination of rational design and random mutagenesis, followed by selection for activity, specificity, multiplexing (and any other desired trait) using specialized selection assays.

FIG. 2: A schematic of the ELANE gene and target sites used to test activity and fidelity of wild-type OMNI-50 nuclease and its variants. “Alt” and “ref” indicate guide RNA molecules that target a nuclease to a SNP position in an allele having either the “alt” version of the SNP or having the “ref” version of the SNP, respectively.

FIGS. 3A-3B: Allele specificity and off-target effects of wild-type OMNI-50 nuclease, demonstrating that the OMNI-50 nuclease could be improved by optimization. FIG. 3A: HSCs edited by wild-type OMNI-50 alt composition showed elimination of the alternative allele while the reference allele was kept intact. However, editing by wild-type OMNI-50 ref composition resulted in a reduction in the reference allele and only partial preservation of the alternative allele, providing a differential but not complete allele specific editing. FIG. 3B: rhAmpseq analysis in healthy (HD) and Patient (ESCN-2)-edited HSCs (RNP(ref) or RNP(alt)) on off-targets identified for constant guide (Sg(constant)), reference guide (Sg(ref)) and alternative guide (Sg(alt)). Results indicate that wild-type OMNI-50 has one off-target for each guide that should be eliminated to ensure target-specific editing. % Editing in RNPref treated HSCs is shown in the top panel; % Editing in RNPalt treated HSCs is shown in the bottom panel. Statistical significance is indicated as **P<0.01, ****P<0.0001.

FIGS. 4A-4C: Optimized variants show improved allele specificity and comparable activity compared to wild-type OMNI-50 nuclease. FIG. 4A: Allele specificity was determined in healthy HSCs edited with wild-type OMNI-50 or optimized variants. Variants show superior allele discrimination compared with wild-type OMNI-50. Specifically, a graph representing allele specific editing with sgRNA-564DS-ref or sgRNA-564DS-alt measured by ddPCR is shown. Editing specificity is determined by two competitive probes, FAM probe which binds the alternative allele and HEX probe which binds the reference allele, governing decrease signal from the edited allele. The ratio between the concentration of the reference allele to the alternative allele in heterozygote untreated cells is 1. The graph represents average concentration of reference allele (HEX), alternative allele (FAM), normalized to endogenous gene control RPP30 and STAT1 for each gDNA sample. FIG. 4B: % excision was measured in healthy HSCs edited with wild-type OMNI-50 (WT OMNI-50) or optimized variants. Variant 3795 shows comparable activity compared with WT OMNI-50. Variant 3795 showed the highest allele specificity without compromising on activity. FIG. 4C: Variant fidelity was measured by next-generation sequence (NGS) analysis specific to known OMNI-50 nuclease off-target. Note, all variants depicted high fidelity.

FIGS. 5A-5C: rhAmpSeq analysis of healthy HSCs edited using either a WT OMNI-50 or the Variant 3795 nuclease on off-targets identified for the constant guide (Sg(constant)), reference guide (Sg(ref)) (RNP(ref), FIG. 5A), and alternative guide (Sg(alt)) molecules (RNP(alt)), FIG. 5B). FIG. 5C shows additional results validating the fidelity of the Variant 3795 nuclease are demonstrated by examining the off-targets when using three different guide molecules (g35, g62Ref, or g62Alt) on HSC samples with either a WT OMNI-50 or Variant 3795 nuclease (FIG. 5C). These results show that there is a strong validated off-target effect for each guide when using the WT OMNI-50 nuclease, but no off-targets were validated with the Variant 3795 nuclease, indicating that the Variant 3795 nuclease eliminates off-targets compared to WT OMNI-50 nuclease.

FIGS. 6A-6C: Mono-allelic excision in ELANE restores neutrophil differentiation. FIG. 6A: A schematic of gene editing treatment to restore neutrophil differentiation. FIG. 6B: Flow cytometry analysis showing Healthy donor (HD-2) and Patient-derived (ESCN-2) HSCs that were edited ex-vivo only on the targeted mutated allele and differentiated into mature neutrophils. FIG. 6C: Quantification of data shown in FIG. 6B.

FIGS. 7A-7B: Linear representation of ELANE's five exons and four introns showing location of representative heterozygous mutations associated with SCN depicted as black inverted triangles (FIG. 7A). Based on Makaryan et al. FIG. 7B: Schematic of three identified SNPs (white inverted triangles), associated with the majority of ELANE mutations and a common cut site (gray inverted triangle), based on which three allele specific sgRNA guides and a constant guide were designed representing three mono-allelic excision strategies (I-III).

FIGS. 8A-8H: Allele specificity and excision efficiency of OMNI Variant 3795 nuclease compositions. FIG. 8A: Scheme depicting experimental workflow. HSCs from healthy donors and SCN patients were electroporated with RNPs or left non-treated followed by 3 days recovery in CD34⁺ expansion media. Cells were then subjected to differentiation by culturing for 7 days with IL-3, SCF, GMCSF and GCSF for proliferation and myeloid progenitor differentiation, followed by a 7-days culture in GCSF for neutrophil differentiation. FIG. 8B: Bar graphs representing percentages of un-edited reference (black) and alternative (gray) alleles at day 6 of differentiation in HSCs taken from either healthy donor (HD-V3) or SCN patient (SCN-P41) treated with RNP (ref) composition or left non-treated (NT), as measured by ddPCR. Average of each allele concentration was normalized to endogenous gene control RPP30 and STAT1 and presented relatively to non-treated cells. (n=3 groups of cells from HD-V3 healthy/SCN-P41 patient donors). Statistical significance is indicated as ****P<0.0001, ns=Not statistically significant. FIG. 8C: Bar graphs representing percentages of excision at days 6 and 14 of differentiation in HSCs taken from either healthy donor (HD-V3): non-treated (NT, black) or RNP (ref)-treated (gray) or SCN patient (SCN-P41): non-treated (NT, white) or RNP (ref)-treated (dark oblique lines), as measured by ddPCR. (n=3 groups of cells from HD-V3 healthy/SCN-P41 patient donors). Statistical significance is indicated as **P<0.01, ****P<0.0001. FIG. 8D: Bar graphs representing percentages of wild-type (black) and mutated (gray) alleles in cDNA taken from SCN-P41 patient HSCs that were either treated with RNP (ref) composition or left non-treated (NT), as measured by next-generation sequencing (NGS) targeting the mutation site. (n=3 groups of cells from SCN-P41 patient). Statistical significance is indicated as ***P<0.001. FIG. 8E: Bar graphs representing percentages of un-edited reference (black) and alternative (gray) alleles at day 6 of differentiation in HSCs taken from SCN patient (SCN-P55) treated with RNP (alt) composition or left non-treated (NT), as measured by ddPCR. Average of each allele concentration was normalized to endogenous gene control RPP30 and STAT1 and presented relatively to non-treated cells. (n=3 groups of cells from SCN-P55 patient donor). Statistical significance is indicated as ****P<0.0001, ns=Not statistically significant. FIG. 8F: Bar graphs representing percentages of excision at days 6 and 14 of differentiation in HSCs taken from SCN patient (SCN-P55): non-treated (NT, white) or RNP (alt)-treated (dark oblique lines), as measured by ddPCR. (n=3 groups of cells from SCN-P55 patient donor). Statistical significance is indicated as ***P<0.001,****P<0.0001. FIG. 8G: Bar graphs representing percentages of wild-type (black) and mutated (gray) alleles in cDNA taken from SCN-P55 patient HSCs that were either treated with RNP (alt) composition or left non-treated (NT), as measured by NGS targeting the mutation site. (n=3 groups of cells from SCN-P55 patient). Statistical significance is indicated as ***P<0.001. FIG. 8H: Bar graphs representing ELANE mRNA levels in day 6 differentiated HSCs of SCN-P41 and SCN-P55 patients that were either not-treated (NT, Black) or RNP(ref)/RNP(alt)-treated, respectively (Excised, Gray). Data is presented relatively to the NT group. (n=3 groups of cells from SCN-P41 and SCN-P55 patients). Statistical significance is indicated as *P<0.05. Bars represent mean values with standard deviation. Bars represent mean values with standard deviation.

FIGS. 9A-9K: OMNI Variant 3795-facilitated editing boosts neutrophil differentiation and maturation in vitro. FIG. 9A: Representative FACS plots of non-treated (NT, left panel) and RNP(ref)-treated (right panel) healthy donor (HD-V3, upper panel) and SCN-P41 patient (lower panel) differentiated HSCs, analyzed for neutrophilic (CD66b+) and monocytic (CD14+/CD66b−) subsets. FIG. 9B: Quantitative analysis of respective FACS data for percentages of neutrophils (CD66b+ cells) in healthy (HD-V3) and SCN patient (SCN-P41) differentiated HSCs that were non-treated (NT, black) or treated with RNP(ref) (gray). (n=3 groups of cells from HD-V3 healthy/SCN-P41 patient donors). Statistical significance is indicated as ****P<0.0001, ns=Not statistically significant. FIG. 9C: Quantitative analysis of respective FACS data for percentages of monocytes (CD14+/CD66b− cells) in healthy (HD-V3) and SCN patient (SCN-P41) differentiated HSCs that were non-treated (NT, black) or treated with RNP(ref) (gray). (n=3 groups of cells from HD-V3 healthy/SCN-P41 patient donors). Statistical significance is indicated as ****P<0.0001, ns=Not statistically significant. FIG. 9D: Quantification of percentages of Zymosan Green uptake by healthy (HD-V3) and SCN patient (SCN-P41) differentiated HSCs that were non-treated (NT, black) or treated with RNP(ref) (gray). ns=Not statistically significant. FIG. 9E: Graph depicts real time change in light emission, relative light units (RLUs), from 200,000 Luciferase expressing bacterial cells incubated with differentiated neutrophils from healthy non-treated (HD-V3, NT; square) or patient RNP(ref)-treated (SCN-P41, RNP(ref); triangle) HSCs compared to bacterial cells only control (e-coli, circle). Statistical significance for each one of the groups versus e-coli control at the last time point presented, when RLU levels reached plateau, is indicated as *P<0.05, **P<0.01. FIG. 9F: Representative FACS plots of non-treated healthy donor (HD-V4 NT, left panel), non-treated SCN patient (SCN-P55 NT, middle panel) and RNP(alt)-treated SCN patient (right panel) differentiated HSCs, analyzed for neutrophilic (CD66b+) and monocytic (CD14+/CD66b−) subsets. FIG. 9G: Quantitative analysis of respective FACS data for percentages of neutrophils (CD66b+ cells) in differentiated HSCs from non-treated healthy donor (HD-V4, NT; black) and SCN patient (SCN-P55) either non-treated (NT, black) or treated with RNP(alt) (gray). (n=3 groups of cells from HD-V4 healthy/SCN-P55 patient donors). Statistical significance is indicated as ****P<0.0001, ns=Not statistically significant. FIG. 9H: Quantitative analysis of respective FACS data for percentages of monocytes (CD14+/CD66b− cells) in differentiated HSCs from non-treated healthy donor (HD-V4, NT; black) and SCN patient (SCN-P55) either non-treated (NT, black) or treated with RNP(alt) (gray). (n=3 groups of cells from HD-V4 healthy/SCN-P55 patient donors). Statistical significance is indicated as ****P<0.0001, ns=Not statistically significant. FIG. 9I: Diff-Quik staining of P55 SCN patient-derived differentiated HSCs treated with RNP(alt) or electroporated without a nuclease composition (SCN-P55 Mock). Microphotographs were taken on LEITZ LABORLUX S polarizing light microscope at 400× magnification using Nikon DSLR digital camera. FIG. 9J: Quantification of percentages of Zymosan Green uptake by differentiated HSCs from non-treated healthy donor (HD-V4, NT; black) and SCN patient (SCN-P55) either non-treated (NT, black) or treated with RNP(alt) (gray). (n=3 groups of cells from HD-V4 healthy/SCN-P55 patient donors). Statistical significance is indicated as *P<0.05. FIG. 9K: Graph depicts real time change in light emission, relative light units (RLUs), from 200,000 Luciferase expressing bacterial cells incubated with differentiated neutrophils from healthy non-treated (HD-V4, NT; square) HSCs and patient RNP(alt)-treated (SCN-P55, RNP(alt); triangle) HSCs compared to bacterial cells only control (E. coli, circle). Statistical significance for each one of the groups versus e-coli control at the last time point presented, when RLU levels reached plateau, is indicated as *P<0.05, ***P<0.001. Bars represent mean values with standard deviation.

FIGS. 10A-10B: Heterozygosity frequency and coverage of patient and healthy populations by the three SNPs. FIG. 10A: Heterozygosity frequency of each of the three chosen SNPs in the healthy (left) or patient (right) population. Heterozygosity frequency was similar among the healthy and patient populations in each of the three SNPs. rs10414837, p value=0.126, Odds ratio=0.653; rs3761005, p value=0.9615, Odds ratio=1.014; rs1683564, p value=0.9475, Odds ratio=1.019, all analyzed by Chi-square. FIG. 10B: Pie chart presenting percentage of the population being heterozygous for at least one of the three chosen SNPs (gray) in the healthy (left) or patient (right) population. Similar coverage of the healthy and patient populations by the three SNPs (more than 75%, p value=0.1285, Odds ratio=0.56, Chi-square).

FIG. 11: Mutation-SNP linkage determination in SCN-P41 and SCN-P55 patients. Electropherograms of sequencing analyses of the mutation site and the rs1683564 SNP. SCN-P41 patient harbors a mutation on the same allele as the reference form of the SNP (C, cytosine), whereas SCN-P55 patient harbors a mutation on the same allele as the alternative form of the SNP (A, adenosine).

FIG. 12: Same editing outcomes with RNP(ref) and RNP(alt) compositions. The ELANE gene is cleaved in two locations: 1) intron 4, a biallelic site guided by sgRNA(constant) guide and 2) a heterozygous SNP site, rs1683564, a single allelic site guided by either sgRNA(ref) or sgRNA(alt) depending on the linkage to the mutation site. If the mutation is located at the allele harboring the reference form of the SNP (C, cytosine), RNP(ref) composition, including a nuclease, sgRNA(ref) and sgRNA(constant), is chosen and a section including the mutation is cleaved from the reference allele (Patient A, upper panel). If the mutation is located at the allele harboring the alternative form of the SNP (A, adenosine), RNP(alt) composition, including a nuclease, sgRNA(alt) and sgRNA(constant), is chosen and a section including the mutation is cleaved from the alternative allele (Patient B, lower panel). Illustration created with BioRender.com.

FIGS. 13A-13C: Inversion events following excision. FIG. 13A: Schematic of detection of inversion events: Specific primers were designed to amplify inverted variations of the excised fragment. EvaGreen dye, a fluorescent DNA-binding dye that binds dsDNA, was used in a ddPCR assay to measure all inversion events. Illustration created with BioRender.com. FIG. 13B: Quantification of total inversion events measured by EvaGreen-based ddPCR assay in unedited (black) and RNP(ref)-treated (gray) HD-V3 healthy donor and SCN-P41 patient derived differentiated HSCs. Statistical significance is indicated as ****P<0.0001. FIG. 13C: Quantification of total inversion events measured by EvaGreen-based ddPCR assay in unedited HD-V4 healthy donor and SCN-P55 patient-derived differentiated HSCs (black) and in RNP(alt)-treated (gray) SCN-P55 patient derived differentiated HSCs. Statistical significance is indicated as ****P<0.0001. Bars represent mean values with standard deviation. (n=3-4 groups of cells from HD-V3 or HD-V4 healthy/SCN-P41 or SCN-P55 patient donors).

FIGS. 14A-14B: Excision levels and allele specificity in additional healthy donors. FIG. 14A: Bar graphs representing percentages of excision in HSCs taken from healthy donors that were either non-treated (NT, black), RNP (ref)-treated (gray) or RNP(alt)-treated (white) as measured by ddPCR. (n=4 groups of cells from 2 healthy donors in each group). Statistical significance is indicated as ****P<0.0001. FIG. 14B: Bar graphs representing percentages of un-edited reference (black) and alternative (gray) alleles in HSCs taken from healthy donors that were non-treated (NT), treated with RNP(ref) or RNP(alt), as measured by ddPCR. Average of each allele concentration was normalized to endogenous gene control RPP30 and STAT1 and presented relatively to non-treated cells. (n=4 groups of cells from two (2) healthy donors in each group). Statistical significance is indicated as ****P<0.0001, ns=Not statistically significant. Bars represent mean values with standard deviation.

FIGS. 15A-15C: Excision levels in Long Term HSC population. FIG. 15A: Representative FACS plots of healthy donor derived CD34⁺ HSCs prior to sorting (Total, left panel) and following sorting to CD90⁻ (middle panel) and CD90⁺ (right panel) populations. (FIG. 15B and FIG. 15C) Bar graphs representing percentages of excision from HSCs taken from two healthy donors (MLP1; FIG. 15B—heterozygous to the alternative form of the SNP and MLP2, FIG. 15C—homozygous to the alternative form of the SNP) prior to sorting (Total, black) and following sorting to CD90⁺ (light gray) and CD90⁻ (dark gray) populations as measured by ddPCR. Non-treated HSCs prior to sorting served as control (NT) (n=2 groups of cells from each healthy donor in each group). Bars represent mean values with standard deviation.

FIGS. 16A-16D: Differentiation into CD11b⁺/CD15⁺ neutrophils. FIG. 16A: Representative FACS plots of non-treated (NT, left panel) and RNP(ref)-treated (right panel) healthy donor (HD-V3, upper panel) and SCN-P41 patient (lower panel) differentiated HSCs, analyzed for neutrophilic (CD11b⁺/CD15⁺) subset. FIG. 16B: Quantitative analysis of respective FACS data for percentages of neutrophils (CD11b⁺/CD15⁺ cells) in healthy (HD-V3) and SCN patient (SCN-P41) differentiated HSCs that were non-treated (NT, black) or treated with RNP(ref) (gray). (n=3 groups of cells from HD-V3 healthy/SCN-P41 patient donors). Statistical significance is indicated as ****P<0.0001, ns=Not statistically significant. FIG. 16C: Representative FACS plots of non-treated healthy donor (HD-V4 NT, left panel), non-treated SCN patient (SCN-P55 NT, middle panel) and RNP(alt)-treated SCN patient (right panel) differentiated HSCs, analyzed for neutrophilic (CD11b⁺/CD15⁺) subset. FIG. 16D: Quantitative analysis of respective FACS data for percentages of neutrophils (CD11b⁺/CD15⁺ cells) in differentiated HSCs from non-treated healthy donor (HD-V4, NT; black) and SCN patient (SCN-P55) either non-treated (NT, black) or treated with RNP(alt) (gray). (n=3 groups of cells from HD-V4 healthy/SCN-P55 patient donors). Statistical significance is indicated as ****P<0.0001. Bars represent mean values with standard deviation.

FIGS. 17A-17B. OMNI-50 variants editing activity of ref and alt allele of g62 in LCL cells. Editing activity was determined by NGS analysis. Displayed average and standard deviation of three replicates.

FIGS. 18A-18B: OMNI-50 variants editing activity of g62 off-targets. Two different off-targets were tested: g62 OT1 and g62 OT2. Displayed average and standard deviation of three replicates.

FIGS. 19A-19B: Specificity of probes and guides. FIG. 19A: The binding of each probe (FAM, black; HEX, gray) to DNA extracted from healthy donor (HD) cells that are homozygous to either the reference or alternative forms of the SNP, was measured by ddPCR. Bar graphs representing concentration of positive events. (n=3 groups of cells). Statistical significance is indicated as ****P<0.0001. FIG. 19B: Bar graphs representing percentages of excision in a healthy donor HSCs that are homozygous to the reference form of the rs1683564 SNP and were either not-treated (NT), treated with RNP(alt) or RNP(ref) as measured by ddPCR. (n=3 groups of cells). Statistical significance is indicated as ****P<0.0001, ns=Not statistically significant. Bars represent mean values with standard deviation.

FIGS. 20A-20D: No detected off targets following editing of OMNI Variant 3795 nuclease and each of the sgRNAs. (FIGS. 20A-C) An unbiased survey (GUIDE-seq) of whole-genome off-target cleavage using OMNI Variant 3795 nuclease and each of the constant guide (FIG. 20A, SgRNA(constant)), reference guide (FIG. 20B, SgRNA(ref)) and alternative guide (FIG. 20C, SgRNA(alt)), showing all reads are of the target sequence and no off-targets detected (4 mismatches). Note, analysis was done in U2OS cells that are homozygous to the reference form of rs1683564 SNP. Since OMNI Variant 3795 nuclease is highly allele discriminatory, when using sgRNA(alt) there is only minor on-target editing of the reference allele (13 reads of the reference cytosine genotype) and no detectable off targets. (FIG. 20D) A table summarizing the results of an in-silico off-target analysis for constant, alternative and reference guides depicting a few potential off-targets. None of these off targets were validated by rhAmpSeq analysis performed on HSCs derived from SCN-P41 and SCN-P55 patients edited with RNP(ref) and RNP(alt), respectively, see two right columns. rhAmpSeq validation threshold was set to editing ≥0.2%.

FIGS. 21A-21J. Excision using RNP(ref) in SCN-P42 and HD-V5. (FIG. 21A) Bar graphs representing percentages of un-edited reference (black) and alternative (gray) alleles at day 6 of differentiation in HSCs taken from either healthy donor (HD-V5) or SCN patient (SCN-P42) treated with RNP (ref) composition or electroporated without a nuclease composition (Mock), as measured by ddPCR. (n=3 groups of cells from HD-V5 healthy/SCN-P42 patient donors). Statistical significance is indicated as ****P<0.0001, ns=Not statistically significant. (FIG. 21B) Bar graphs representing percentages of excision at days 6 and 14 of differentiation in HSCs taken from either healthy donor (HD-V5): Mock-treated (Mock, black) or RNP (ref)-treated (gray), or SCN patient (SCN-P42): Mock-treated (Mock, white) or RNP (ref)-treated (dark oblique lines), as measured by ddPCR. (n=3 groups of cells from HD-V5 healthy/SCN-P42 patient donors). Statistical significance is indicated as ****P<0.0001. (FIG. 21C) Bar graphs representing ELANE mRNA levels in day 6 differentiated HSCs of SCN-P42 patient that were either Mock-treated (Black) or RNP(ref)-treated (Gray). Data is presented relatively to the mock group. (n=3 groups of cells from SCN-P42 patient). Statistical significance is indicated as **P<0.01. (FIG. 21D) Bar graphs representing percentages of wild-type (black) and mutated (gray) alleles in cDNA taken from SCN-P42 patient HSCs that were either RNP (ref)-treated or Mock-treated (Mock), as measured by NGS targeting the mutation site. (n=3 groups of cells from SCN-P42 patient). Statistical significance is indicated as *P<0.05. (FIG. 21E) Representative FACS plots of mock-treated (Mock, left panel) and RNP(ref)-treated (right panel) healthy donor (HD-V5, upper panel) and SCN-P42 patient (lower panel) differentiated HSCs, analyzed for neutrophilic (CD66b+) and monocytic (CD14⁺/CD66b⁻) subsets. (FIG. 21F) Quantitative analysis of respective FACS data for percentages of neutrophils (CD66b+ cells) in healthy (HD-V5) and SCN patient (SCN-P42) differentiated HSCs that were mock-treated (Mock, black) or treated with RNP(ref) (gray). (n=3 groups of cells from HD-V5 healthy/SCN-P42 patient donors). Statistical significance is indicated as ****P<0.0001, ns=Not statistically significant. (FIG. 21G) Quantitative analysis of respective FACS data for percentages of monocytes (CD14⁺/CD66b⁻ cells) in healthy (HD-V5) and SCN patient (SCN-P42) differentiated HSCs that were mock-treated (Mock, black) or treated with RNP(ref) (gray). (n=3 groups of cells from HD-V5 healthy/SCN-P42 patient donors). Statistical significance is indicated as ****P<0.0001, ns=Not statistically significant. (FIG. 21H) Diff-Quik staining of P42 SCN patient-derived differentiated HSCs treated with RNP(ref) or electroporated without a nuclease composition (SCN-P42 Mock). Microphotographs were taken on LEITZ LABORLUX S polarizing light microscope at 400× magnification using Nikon DSLR digital camera. (FIG. 21I) Quantification of percentages of Zymosan Green uptake by healthy (HD-V5) and SCN patient (SCN-P42) differentiated HSCs that were mock-treated (Mock, black) or treated with RNP(ref) (gray). Statistical significance is indicated as ****P<0.0001, ns=Not statistically significant. (FIG. 21J) Graph depicts real time change in light emission, relative light units (RLUs), from 200,000 Luciferase expressing bacterial cells incubated with differentiated neutrophils from healthy mock-treated (HD-V5, Mock; white square), patient mock-treated (SCN-42 Mock; crossed circle) or patient RNP(ref)-treated (SCN-P42 RNP(ref); triangle) HSCs compared to bacterial cells only control (e-coli, circle). Statistical significance for each one of the groups versus e-coli control at the last time point presented, when RLU levels reached plateau, is indicated as ****P<0.0001. Bars represent mean values with standard deviation.

FIGS. 22A-22I: Excision using RNP(alt) in SCN-P12 and HD-V1. (FIG. 22A) Bar graphs representing percentages of un-edited reference (black) and alternative (gray) alleles at day 6 of differentiation in HSCs taken from either healthy donor (HD-V1) or SCN patient (SCN-P12) treated with RNP (alt) composition or electroporated without a nuclease composition (Mock), as measured by ddPCR. (n=3 groups of cells from HD-V1 healthy/SCN-P12 patient donors). Statistical significance is indicated as ****P<0.0001, ns=Not statistically significant. (FIG. 22B) Bar graphs representing percentages of excision at days 6 and 14 of differentiation in HSCs taken from either healthy donor (HD-V1): Mock-treated (Mock, black) or RNP (alt)-treated (gray), or SCN patient (SCN-P12): Mock-treated (Mock, white) or RNP (alt)-treated (dark oblique lines), as measured by ddPCR. (n=3 groups of cells from HD-V1 healthy/SCN-P12 patient donors). Statistical significance is indicated as ***P<0.001, ****P<0.0001. (FIG. 22C) Bar graphs representing ELANE mRNA levels in day 6 differentiated HSCs of SCN-P12 patient that were either Mock-treated (Black) or RNP(alt)-treated (Gray). Data is presented relatively to the mock group. (n=3 groups of cells from SCN-P12 patient). Statistical significance is indicated as *P<0.05. (FIG. 22D) Bar graphs representing percentages of wild-type (black) and mutated (gray) alleles in cDNA taken from SCN-P12 patient HSCs that were either RNP (alt)-treated or Mock-treated (Mock), as measured by NGS targeting the mutation site. (n=3 groups of cells from SCN-P12 patient). Statistical significance is indicated as ***P<0.001. (FIG. 22E) Representative FACS plots of mock-treated (Mock, left panel) and RNP(alt)-treated (right panel) healthy donor (HD-V1, upper panel) and SCN-P12 patient (lower panel) differentiated HSCs, analyzed for neutrophilic (CD66b+) and monocytic (CD14⁺/CD66b⁻) subsets. (FIG. 22F) Quantitative analysis of respective FACS data for percentages of neutrophils (CD66b+cells) in healthy (HD-V1) and SCN patient (SCN-P12) differentiated HSCs that were mock-treated (Mock, black) or treated with RNP(alt) (gray). (n=3 groups of cells from HD-V1 healthy/SCN-P12 patient donors). Statistical significance is indicated as **P<0.01, ***P<0.001. (FIG. 22G) Quantitative analysis of respective FACS data for percentages of monocytes (CD14+/CD66b−cells) in healthy (HD-V1) and SCN patient (SCN-P12) differentiated HSCs that were mock-treated (Mock, black) or treated with RNP(alt) (gray). (n=3 groups of cells from HD-V1 healthy/SCN-P12 patient donors). Statistical significance is indicated as ****P<0.0001, ns=Not statistically significant. (FIG. 22H) Quantification of percentages of Zymosan Green uptake by healthy (HD-V1) and SCN patient (SCN-P12) differentiated HSCs that were mock-treated (Mock, black) or treated with RNP(alt) (gray). Statistical significance is indicated as ***P<0.001, ns=Not statistically significant. (FIG. 22I) Graph depicts real time change in light emission, relative light units (RLUs), from 200,000 Luciferase expressing bacterial cells incubated with differentiated neutrophils from healthy mock-treated (HD-V1, Mock; white square), patient mock-treated (SCN-12 Mock; crossed circle) or patient RNP(alt)-treated (SCN-P12 RNP(alt); triangle) HSCs compared to bacterial cells only control (e-coli, circle). Statistical significance for each one of the groups versus e-coli control at the last time point presented, when RLU levels reached plateau, is indicated as *P<0.05, **P<0.01, ***P<0.001. Bars represent mean values with standard deviation.

FIGS. 23A-23I: Excision using RNP(alt) in SCN-P56 and HD-V3. (FIG. 23A) Bar graphs representing percentages of un-edited reference (black) and alternative (gray) alleles at day 6 of differentiation in HSCs taken from either healthy donor (HD-V3) or SCN patient (SCN-P56) treated with RNP (alt) composition or electroporated without a nuclease composition (Mock), as measured by ddPCR. (n=3 groups of cells from HD-V3 healthy/SCN-P56 patient donors). Statistical significance is indicated as ****P<0.0001, ns=Not statistically significant. (FIG. 23B) Bar graphs representing percentages of excision at day 6 of differentiation in HSCs taken from either healthy donor (HD-V3): Mock-treated (Mock, black) or RNP (alt)-treated (gray), or SCN patient (SCN-P56): Mock-treated (Mock, white) or RNP (alt)-treated (dark oblique lines), as measured by ddPCR. (n=3 groups of cells from HD-V3 healthy/SCN-P56 patient donors). Statistical significance is indicated as ***P<0.001. (FIG. 23C) Bar graphs representing percentages of wild-type (black) and mutated (gray) alleles in cDNA taken from SCN-P56 patient HSCs that were either RNP (alt)-treated or Mock-treated (Mock), as measured by NGS targeting the mutation site. (n=3 groups of cells from SCN-P56 patient). Statistical significance is indicated as ****P<0.0001. (FIG. 23D) Representative FACS plots of mock-treated (Mock, left panel) and RNP(alt)-treated (right panel) healthy donor (HD-V3, upper panel) and SCN-P56 patient (lower panel) differentiated HSCs, analyzed for neutrophilic (CD66b+) and monocytic (CD14⁺/CD66b⁻) subsets. (FIG. 23E) Quantitative analysis of respective FACS data for percentages of neutrophils (CD66b+ cells) in healthy (HD-V3) and SCN patient (SCN-P56) differentiated HSCs that were mock-treated (Mock, black) or treated with RNP(alt) (gray). (n=3 groups of cells from HD-V3 healthy/SCN-P56 patient donors). Statistical significance is indicated as ****P<0.0001, ns=Not statistically significant. (FIG. 23F) Quantitative analysis of respective FACS data for percentages of monocytes (CD14⁺/CD66b⁻ cells) in healthy (HD-V3) and SCN patient (SCN-P56) differentiated HSCs that were mock-treated (Mock, black) or treated with RNP(alt) (gray). (n=3 groups of cells from HD-V3 healthy/SCN-P56 patient donors). Statistical significance is indicated as P=0.04, ****P<0.0001. (FIG. 23G) Diff-Quik staining of P56 SCN patient-derived differentiated HSCs treated with RNP(alt) or electroporated without a nuclease composition (SCN-P56 Mock). Microphotographs were taken on LEITZ LABORLUX S polarizing light microscope at 400× magnification using Nikon DSLR digital camera. (FIG. 23H) Quantification of percentages of Zymosan Green uptake by healthy (HD-V3) and SCN patient (SCN-P56) differentiated HSCs that were mock-treated (Mock, black) or treated with RNP(alt) (gray). Statistical significance is indicated as ****P<0.0001, ns=Not statistically significant. (FIG. 23I) Graph depicts real time change in light emission, relative light units (RLUs), from 200,000 Luciferase expressing bacterial cells incubated with differentiated neutrophils from healthy mock-treated (HD-V3, Mock; white square), patient mock-treated (SCN-56 Mock; crossed circle) or patient RNP(alt)-treated (SCN-P56 RNP(alt); triangle) HSCs compared to bacterial cells only control (e-coli, circle). Statistical significance for each one of the groups versus e-coli control at the last time point presented, when RLU levels reached plateau, is indicated as **P<0.01. Bars represent mean values with standard deviation.

### DETAILED DESCRIPTION

The present disclosure provides an engineered OMNI-50 nuclease exhibiting increased specificity to a target site compared to the wild-type OMNI-50 nuclease (SEQ ID NO: 1). The wild-type OMNI-50 nuclease is disclosed in PCT International Application Publication No. WO/2020-030782, incorporated herein by reference. When the engineered OMNI-50 nuclease variant is active in a CRISPR endonuclease system, the CRISPR endonuclease system displays reduced off-target editing activity and maintained on-target editing activity relative to a CRISPR endonuclease system comprising the wild-type OMNI-50 nuclease. In some embodiments, the engineered OMNI-50 nuclease is an OMNI-50 nuclease variant comprising at least one amino acid substitution relative to the wild-type OMNI-50 nuclease. In some embodiments, the engineered OMNI-50 nuclease comprises multiple amino acid substitutions compared to wild-type OMNI-50 nuclease.

In some embodiments, and OMNI-50 nuclease variant is at least 80%, e.g., at least 85%, 86%, 87%, 88%, 89%, 90%, 91%, 92%, 93%, 94%, 95%, 96%, 97%, 98% or 99% identical to the amino acid sequence of SEQ ID NO: 1. As a non-limiting example, an OMNI-50 nuclease variant may have amino acid sequence differences at up to 1%, 2%, 3%, 4%, 5%, 6%, 7%, 8%, 9%, 10%, 11%, 12%, 13%, 14%, 15%, or 20% of its residues relative to SEQ ID NO:1. Such sequence differences may be revealed by a sequence alignment. An OMNI-50 variant nuclease may be generated by replacing at least one amino acid residue of an OMNI-50 wild-type nuclease with another amino acid residue e.g. with a conservative or non-conservative amino acid substitution, and/or by inserting or deleting an amino acid residue of the OMNI-50 wild-type nuclease. Any such mutations, including but not limited to substitutions, insertions, or deletions, in addition to any other mutations described herein, or with mutations in addition to the mutations described herein, may be used to generate an OMNI-50 variant nuclease from an OMNI-50 wild-type nuclease. In some embodiments, the OMNI-50 variant nuclease retains a desired activity of the parent wild-type OMNI-50 nuclease, e.g., the ability to interact with a guide RNA and target DNA and/or the activity of the nuclease (e.g. ability to cause a double-strand DNA break, a single-strand DNA break, or lack of any nuclease or nickase activity). In some embodiments, the variant retains the desired activity of the parent, e.g. nuclease activity, at a level greater than or equal to the level of activity of the parent. In some embodiments, the variant retains the desired activity of the parent at a level of at least 100%, 95%, 90%, 80%, 70%, 60%, 50%, 40%, or 30% the level of activity of the parent. In some embodiments, the OMNI-50 variant nuclease displays reduced off-target effects relative to OMNI-50 wild-type nuclease.

In some embodiments, there is provided a variant of OMNI-50 nuclease protein comprising a sequence that is at least 80% identical to the amino acid sequence of wild-type OMNI-50 (SEQ ID NO: 1) and having at least one amino acid substitution. In some embodiments, the amino acid substitution comprises an amino acid residue replacement to a positive, negative, uncharged, hydrophilic, hydrophobic, polar, or non-polar amino acid. In some embodiments, the amino acid substitution is selected from replacement of an amino acid to any one of a different amino acid selected from the group consisting of R, K, H, D, E, S, T, N, Q, C, U, G, P, A, I, L, M, F, W, Y and V.

Positive amino acids include any amino acid having a positively charged R-group, e.g. lysine (K), arginine (R), or Histidine (H). Negative amino acids include any amino acid having a negatively charged R-group, e.g. aspartic acid (D) or glutamic acid (E). Uncharged amino acids or neutral amino acids include amino acids whose R-group does not normally carry a charge. Polar amino acids include any amino acid having a polar R-group, e.g. serine (S), threonine (T), tyrosine (Y), asparagine (N), or glutamine (Q). Non-polar amino acids include any amino acid having a non-polar R-group, e.g. glycine (G), alanine (A), valine (V), cysteine (C), proline (P), leucine (L), isoleucine (I), methionine (M), tryptophan (W), or phenylalanine (F).

Properties of an original variant protein having an original amino acid substitution at a given position may be extended to a different variant having a different amino acid substitution at the same position if the different amino acid substitution has an R-group with similar properties to the original amino acid substitution. For example, if a variant protein is shown to have higher specificity compared to a wild-type protein by substituting a glutamic acid (E) residue for a lysine (K) residue, it is reasonable to consider that a similar variant substituting the glutamic acid (E) residue for an arginine (R) residue will also display higher specificity since both lysine (K) and arginine (R) share similar properties (e.g. they both contain positively charged R-groups). Conversely, a variant having a substitution of the glutamic acid (E) residue to an aspartic acid (D) is less likely to display the higher specificity property because both glutamic acid (E) and aspartic acid (D) share the similar property of both containing a negatively charged R-group.

In some embodiments, a variant OMNI-50 nuclease protein contains an amino acid substitution in at least one of the following positions in the wild-type OMNI-50 protein sequence (SEQ ID NO:1): R61, Y437, R478, A493, Y545, G606, K688, L690, E695, L718, R788, Q803, L805, L844, K965, V981, and K1036. Each possibility represents a separate embodiment of the present disclosure. In some embodiments, there is provided a variant of OMNI-50 nuclease protein comprising a sequence that is at least 80% identical to the amino acid sequence of the wild-type OMNI-50 nuclease (SEQ ID NO: 1) and having at least one amino acid substitution in at least one of the following positions in the wild-type OMNI-50 protein sequence: R61, Y437, R478, A493, Y545, G606, K688, L690, E695, L718, R788, Q803, L805, L844, K965, V981, and K1036. In some embodiments, the variant OMNI-50 nuclease protein comprises at least one of the following amino acid substitutions in the following positions in the wild-type OMNI-50 protein sequence: R478I, Y545H, Q803V, L805I, R61A, Y437W, R788K, L844N, V981M, G606P, L690V, E695Q, R478T, A493G, K688E, L718C, K965V, and K1036V. Each possibility represents a separate embodiment of the present disclosure. In some embodiments, the substitution corresponds to the mutations listed in Table 1.

In some embodiments, the variant OMNI-50 nuclease protein comprises at least one amino acid substitution in the following positions in the wild-type OMNI-50 protein sequence: R478, Y545, Q803, and L805. In some embodiments, the variant OMNI-50 nuclease protein comprises amino acid substitutions in the following positions in the wild-type OMNI-50 protein sequence: R478, Y545, Q803, and L805. In some embodiments, the variant OMNI-50 nuclease protein comprises the following amino acid substitutions from the wild-type OMNI-50 protein sequence: R478I, Y545H, Q803V, and L805I.

In some embodiments, the variant OMNI-50 nuclease protein comprises at least one amino acid substitution in the following positions in the wild-type OMNI-50 protein sequence: R61, Y437, R788, L844, and V981. In some embodiments, the variant OMNI-50 nuclease protein comprises amino acid substitutions in the following positions in the wild-type OMNI-50 protein sequence: R61, Y437, R788, L844, and V981. In some embodiments, the variant OMNI-50 nuclease protein comprises the following amino acid substitutions from the wild-type OMNI-50 protein sequence: R61A, Y437W, R788K, L844N, and V981M.

In some embodiments, the variant OMNI-50 nuclease protein comprises at least one amino acid substitution in the following positions in the wild-type OMNI-50 protein sequence: G606, L690, and E695. In some embodiments, the variant OMNI-50 nuclease protein comprises amino acid substitutions in the following positions in the wild-type OMNI-50 protein sequence: G606, L690, and E695. In some embodiments, the variant OMNI-50 nuclease protein comprises the following amino acid substitutions from the wild-type OMNI-50 protein sequence: G606P, L690V, and E695Q.

In some embodiments, the variant OMNI-50 nuclease protein comprises at least one amino acid substitution in the following positions in the wild-type OMNI-50 protein sequence: R478, A493, K688, L718, K965, and K1036. In some embodiments, the variant OMNI-50 nuclease protein comprises amino acid substitutions in the following positions in the wild-type OMNI-50 protein sequence: R478, A493, K688, L718, K965V, and K1036. In some embodiments, the variant OMNI-50 nuclease protein comprises the following amino acid substitutions from the wild-type OMNI-50 protein sequence: R478T, A493G, K688E, L718C, K965V, and K1036V.

In some embodiments, the OMNI-50 variant nuclease further comprises one or more of a nuclear localization sequence (NLS), cell penetrating peptide sequence, and/or affinity tag. In an embodiment, the OMNI-50 variant nuclease comprises one or more nuclear localization sequences of sufficient strength to drive accumulation of a CRISPR complex comprising the CRISPR nuclease in a detectable amount in the nucleus of a eukaryotic cell.

In some embodiments, the OMNI-50 variant nuclease comprises amino acid substitutions selected from amino acid substitutions corresponding to the substitutions displayed relative to wild-type OMNI-50 in Table 1.

According to some embodiments, there is provided an isolated OMNI-50 variant nuclease protein comprising one or more substitutions or mutations relative to the wild-type OMNI-50 nuclease sequence, wherein the isolated variant OMNI-50 variant nuclease is active in a CRISPR system, wherein the CRISPR system displays reduced off-target editing activity and maintained on-target editing activity relative to a wild-type CRISPR system.

According to some embodiments, additional mutations to the OMNI-50 variant nuclease described herein may be implemented. Examples include, but are not limited to, mutations which alter the PAM recognition sequence, alter the nuclease activity of the enzyme, and truncations or removal of portions of the nuclease. According to some embodiments, the variant OMNI-50 variant nuclease may be encoded by any nucleic acid sequence which produces the desired amino acid sequence of the variant. For example, the nuclei acid sequence may be codon-optimized for a cell, such as a bacterial cell, plant cell, or mammalian cell.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and a single-guide RNA (sgRNA) molecule. Alternatively, a CRISPR nucleases may form a CRISPR complex comprising the CRISPR nuclease, an crRNA molecule, and a tracrRNA molecule.

According to some embodiments of the present invention, there is provided a method of gene editing having reduced off-target editing activity and/or increased on-target editing activity, comprising: contacting a target site locus with an active CRISPR endonuclease system having a variant OMNI-50 protein complexed with a suitable guide RNA or guide RNA complex, wherein the active CRISPR endonuclease system displays reduced off-target editing activity and maintained on-target editing activity relative to a wild-type OMNI-50 CRISPR system.

According to some embodiments, there is provided a non-naturally occurring OMNI-50 nuclease variant having a wild-type OMNI-50 protein sequence (SEQ ID NO: 1) comprising an amino acid substitution in at least one of the following positions: R61, Y437, R478, A493, Y545, G606, K688, L690, E695, L718, R788, Q803, L805, L844, V981, K965, and K1036.

In some embodiments, the amino acid substitution at position R478 and/or position Y545, preferably at both position R478 and position Y545.

In some embodiments, the OMNI-50 nuclease variant comprises an amino acid substitution at each of positions R478 and Y545.

In some embodiments, the amino acid substitution at position R478 is any one of the following substitutions: R478D, R478E, R478S, R478T, R478N, R478Q, R478G, R478P, R478C, R478A, R478V, R478I, R478L, R478M, R478F, R478Y, or R478W, preferably R478V, R478H, R478L, R478M, R478P, R478F, R478W, R478Y, R478S, R478C, R478T, R478N, or R478Q.

In some embodiments, the amino acid substitution is at position R478 and the amino acid substituted for arginine is an amino acid having a negatively charged R-group or an R-group lacking a charge.

In some embodiments, the amino acid substitution is at position R478 and the amino acid substituted for arginine is a polar amino acid or non-polar amino acid.

In some embodiments, the amino acid substitution is at position R478 and the amino acid substituted for arginine is a non-polar amino acid.

In some embodiments, the amino acid substitution at position Y545 is any one of the following substitutions: Y545D, Y545E, Y545S, Y545T, Y545N, Y545Q, Y545G, Y545P, Y545C, Y545A, Y545V, Y545I, Y545L, Y545M, Y545F, Y545R, Y545K, Y545H, or Y545W, preferably Y545W, Y545F, Y545H, Y545V, or Y545G.

In some embodiments, the amino acid substitution is at position Y545 and the amino acid substituted for tyrosine is an amino acid having a negatively charged R-group or a positively charged R-group.

In some embodiments, the amino acid substitution is at position Y545 and the amino acid substituted for tyrosine is a non-polar amino acid.

In some embodiments, the amino acid substitution is at position Y545 and the amino acid substituted for tyrosine lacks a phenyl ring or is phenylalanine.

In some embodiments, the amino acid substitutions are R478I and Y545H.

In some embodiments, the amino acid substitution is any one of the following substitutions: R478I, Y545H, Q803V, L805I, R61A, Y437W, R788K, L844N, V981M, G606P, L690V, E695Q, R478T, A493G, K688E, L718C, K965V, and K1036V.

In some embodiments, the OMNI-50 nuclease variant comprises an amino acid substitution at each of positions R478, Y545, Q803, and L805.

In some embodiments, the amino acid substitutions are R478I, Y545H, Q803V, and L805I.

In some embodiments, the OMNI-50 nuclease variant comprises an amino acid substitution at each of positions R61, Y437, R788, L844, and V981.

In some embodiments, the amino acid substitutions are R61A, Y437W, R788K, L844N, and V981M.

In some embodiments, the OMNI-50 nuclease variant comprises an amino acid substitution at each of positions G606, L690, and E695.

In some embodiments, the amino acid substitutions are G606P, L690V, and E695Q.

In some embodiments, the OMNI-50 nuclease variant comprises an amino acid substitution at each of positions R478, A493, K688, L718, K965, and K1036.

In some embodiments, the amino acid substitutions are R478T, A493G, K688E, L718C, K965V, and K1036V.

In some embodiments, the OMNI-50 nuclease variant comprises an amino acid substitution at position Q803.

In some embodiments, the amino acid substitution is Q803V.

In some embodiments, the OMNI-50 nuclease variant comprises an amino acid substitution at position Y545.

In some embodiments, the amino acid substitution is Y545H.

In some embodiments, the OMNI-50 nuclease variant comprises an amino acid substitution at position L805.

In some embodiments, the amino acid substitution is L805I.

In some embodiments, the OMNI-50 nuclease variant comprises an amino acid substitution at each of positions R478, Y545, and Q803.

In some embodiments, the amino acid substitutions are R478I, Y545H, and Q803V.

In some embodiments, the OMNI-50 nuclease variant comprises an amino acid substitution at each of positions R478, Y545, and L805.

In some embodiments, the amino acid substitutions are R478I, Y545H, and L805I.

In some embodiments, the OMNI-50 nuclease variant comprises an amino acid substitution at each of positions R478, Q803, and L805.

In some embodiments, the amino acid substitutions are R478I, Q803V, and L805I.

In some embodiments, the OMNI-50 nuclease variant comprises an amino acid substitution at each of positions Y545, Q803, and L805.

In some embodiments, the amino acid substitutions are R478I, Y545H, and L805I.

In some embodiments, the OMNI-50 nuclease variant comprises an amino acid substitution at position R478 and/or position Y545.

In some embodiments, the amino acid substitution at position R478 is any one of the following substitutions: R478D, R478E, R478S, R478T, R478N, R478Q, R478G, R478P, R478C, R478A, R478V, R478I, R478L, R478M, R478F, R478Y, or R478W.

In some embodiments, the amino acid substitution is at position R478 and the amino acid substituted for arginine is an amino acid having a negatively charged R-group or an R-group lacking a charge.

In some embodiments, the amino acid substitution is at position R478 and the amino acid substituted for arginine is a polar amino acid or non-polar amino acid.

In some embodiments, the amino acid substitution is at position R478 and the amino acid substituted for arginine is a non-polar amino acid.

In some embodiments, the amino acid substitution is at position R478 and the amino acid substituted for arginine is selected from a large hydrophobic amino acid (e.g., leucine, methionine, proline, valine), an aromatic amino acid (e.g., histidine, phenylalanine, tryptophan and tyrosine), and a polar uncharged amino acid (e.g., serine, cysteine, threonine, asparagine, and glutamine).

In some embodiments, the amino acid substitution at position Y545 is any one of the following substitutions: Y545D, Y545E, Y545S, Y545T, Y545N, Y545Q, Y545G, Y545P, Y545C, Y545A, Y545V, Y545I, Y545L, Y545M, Y545F, Y545R, Y545K, Y545H, or Y545W.

In some embodiments, the amino acid substitution is at position Y545 and the amino acid substituted for tyrosine is an amino acid having a negatively charged R-group or a positively charged R-group.

In some embodiments, the amino acid substitution is at position Y545 and the amino acid substituted for tyrosine is an amino acid having a positively charged R-group. Non-limiting examples of positively charged amino acids include histidine, lysine and arginine.

In some embodiments, the amino acid substitution is at position Y545 and the amino acid substituted for tyrosine is a non-polar amino acid. Non-limiting examples of non-polar amino acids include glycine, alanine, valine, proline, leucine, isoleucine, methionine, tryptophan and phenylalanine.

In some embodiments, the amino acid substitution is at position Y545 and the amino acid substituted for tyrosine lacks a phenyl ring or is phenylalanine.

In some embodiments, the amino acid substitution is at position Y545 and the amino acid substituted for tyrosine includes a phenyl ring (e.g., phenylalanine).

In some embodiments, the OMNI-50 nuclease variant comprises an amino acid substitution at each of positions R478 and Y545.

In some embodiments, the amino acid substitutions are R478I and Y545H. However, others amino acid substitutions at the R478 and Y545 positions are also contemplated. As a non-limiting example, amino acids having R-groups with similar non-polar to properties to isoleucine, such as alanine (A), valine (V), and leucine (L), are contemplated substitutions at the R478 position. In another non-limiting example, amino acids having R-groups with similar non-polar to properties to isoleucine, such as methionine (M), phenylalanine (F), tyrosine (Y), and tryptophan (W) are contemplated substitutions at the R478 position.

In some embodiments, the OMNI-50 nuclease variant has an amino acid sequence selected from the group consisting of SEQ ID NOs: 2-5, 63-70, and 89-97.

In some embodiments, the OMNI-50 nuclease variant has at least 80% sequence identity to the wild-type OMNI-50 protein sequence (SEQ ID NO:1).

In some embodiments, the OMNI-50 nuclease variant further comprises a nuclear localization sequence (NLS).

In some embodiments, the OMNI-50 nuclease variant exhibits increased specificity toward a DNA target site when complexed with a guide RNA molecule that targets variant to the said DNA target site relative to a wild-type OMNI-50 nuclease complexed with the guide RNA molecule.

According to some embodiments of the present invention, there is provided a CRISPR system comprising any one of the OMNI-50 nuclease variants described herein complexed with a guide RNA molecule that targets a DNA target site, wherein the CRISPR system displays reduced off-target editing activity relative to a wild-type CRISPR system comprising a wild-type OMNI-50 nuclease protein and the guide RNA molecule.

According to some embodiments of the present invention, there is provided a method for gene editing having reduced off-target editing activity, comprising contacting a DNA target site with an active CRISPR system comprising any one of the OMNI-50 nuclease variant proteins described herein.

In some embodiments, the active CRISPR system displays reduced off-target editing activity relative to a wild-type CRISPR system comprising a wild-type OMNI-50 nuclease protein.

In some embodiments, the gene editing occurs in a eukaryotic cell or prokaryotic cell.

In some embodiments, the eukaryotic cell is a plant cell or mammalian cell.

In some embodiments, the mammalian cell is a human cell.

In some embodiments, the DNA target site is located within or in proximity to a pathogenic allele of a gene.

In some embodiments, the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, RPE65, LDLR, ANGPTL3, B2M, TRAC, TCF4, TGFBi, PAX6, C3, LRRK2, SARM1, SAMD9, SAMD9L, HAVCR2, CD3E, APLP2, CISH, TIGIT, TNNT2, TNN, MYH7, and HLA-E.

In some embodiments, the DNA target is repaired with an exogenous donor molecule.

In some embodiments, the off-target editing activity is reduced by at least 2-fold, 10-fold, 10²-fold, 10³-fold, 10⁴-fold, 10⁵-fold, or 10⁶-fold.

According to some embodiments of the present invention, there is provided a modified cell obtained by any one of the methods described herein.

In some embodiments, the cell is capable of engraftment.

In some embodiments, the cell is capable of giving rise to progeny cells after engraftment.

In some embodiments, the cell is capable of giving rise to progeny cells after an autologous engraftment.

In some embodiments, the cell is capable of giving rise to progeny cells for at least 12 months or at least 24 months after engraftment.

In some embodiments, the cell is selected from the group consisting of a hematopoietic stem cell, a progenitor cell, a CD34+ hematopoietic stem cell, a bone marrow cell, and a peripheral mononucleated cell.

According to some embodiments of the present invention, there is provided a composition comprising any one of the modified cells described herein and a pharmaceutically acceptable carrier. According to some embodiments of the present invention, there is provided an in vitro or ex vivo method of preparing the composition, comprising mixing the cells with the pharmaceutically acceptable carrier.

According to some embodiments of the present invention, there is provided a polynucleotide molecule encoding any one of the OMNI-50 variant proteins described herein.

### Delivery

The OMNI-50 variant compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-O-methyl (M), 2′-O-methyl, 3′phosphorothioate (MS) or 2′-O-methyl, 3′thioPACE (MSP), pseudouridine, and 1-methyl pseudo-uridine. Each possibility represents a separate embodiment of the present invention.

The OMNI-50 variants and/or polynucleotides encoding same described herein, and/or additional molecules, such as a single-guide RNA molecule, crRNA molecule, tracrRNA molecules or a nucleotide molecule that encodes any one of them, may be delivered to a target cell by any suitable means. The target cell may be any type of cell e.g., eukaryotic or prokaryotic, in any environment e.g., isolated or not, maintained in culture, in vitro, ex vivo, in vivo or in planta. A target site in a target cell may be within the nucleus of the cell.

The compositions described herein may be introduced into a cell as part of a vector molecule having additional sequences such as, for example, replication origins, promoters and genes encoding antibiotic resistance. Moreover, compositions may introduced into a cell as naked nucleic acids or proteins, as nucleic acids or proteins complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. As non-limiting examples, the composition may be packaged into an adeno-associated virus (AAV), or into a lentivirus, such as a non-integrating lentivirus or a lentivirus lacking reverse transcription capability. Additional non-limiting examples include packaging the composition into liposomes, extracellular vesicles, or exosomes, which may be pseudotyped with vesicular stomatitis glycoprotein (VSVG) or conjugated to a cell-penetrating peptide, an antibody, a targeting moiety, or any combination thereof.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. Optionally the lentivirus includes mRNA of the nuclease and a guide RNA molecule, e.g. a single-guide RNA molecule or crRNA molecule, which is used to target the nuclease to a target site. In some embodiments, the composition delivered to a cell includes mRNA of the nuclease, a guide RNA molecule and a donor template molecule. Optionally, the lentivirus includes the nuclease protein variant and a guide RNA molecule. Optionally, the composition delivered to a cell includes the nuclease protein variant, a guide RNA molecule and/or donor template for homology directed repair. Optionally, the composition delivered to a cell includes mRNA of the nuclease variant, a DNA-targeting crRNA molecule, and a tracrRNA molecule. the composition delivered to a cell includes mRNA of the nuclease variant, DNA-targeting crRNA molecule, and a tracrRNA molecule, and a donor template molecule. the composition delivered to a cell includes the nuclease protein variant, DNA-targeting crRNA molecule, and a tracrRNA molecule. Optionally, the composition delivered to a cell includes the nuclease protein variant, DNA-targeting crRNA molecule, and a tracrRNA molecule, and DNA donor template molecule for homology directed repair.

Any suitable viral vector system may be used to deliver such compositions. Conventional viral and non-viral based gene transfer methods can be used to introduce nucleic acids and/or OMNI-50 variant protein in cells (e.g., mammalian cells, plant cells, etc.) and target tissues. Such methods can also be used to administer nucleic acids encoding and/or OMNI-50 variant protein to cells in vitro. In certain embodiments, nucleic acids and/or a OMNI-50 variant protein are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids and/or proteins include electroporation, lipofection, microinjection, biolistics, particle gun acceleration, virosomes, virus-like particles, exosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al (2009) Nature Biotechnology 27(7) p. 643).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. A OMNI-50 variant or a nucleic acid expressing the variant, as well as any associated nucleic acids, may be delivered by a non-integrating lentivirus. Optionally, RNA delivery with lentivirus is utilized. Optionally, the lentivirus includes mRNA of the nuclease and a guide RNA molecule, e.g. a single-guide RNA molecule or crRNA molecule, which is used to target the nuclease to a target site. Optionally the lentivirus includes mRNA of the nuclease, guide RNA molecule and a donor template molecule. Optionally, the lentivirus includes the nuclease protein variant and a guide RNA molecule. Optionally, the lentivirus includes the nuclease protein variant, a guide RNA molecule and/or donor template molecule for homology directed repair. Optionally, the lentivirus includes mRNA of the nuclease variant, a DNA-targeting crRNA molecule, and a tracrRNA molecule. Optionally the lentivirus includes mRNA of the nuclease variant, DNA-targeting crRNA molecule, and a tracrRNA molecule, and a donor template molecule. Optionally, the lentivirus includes the nuclease protein variant, DNA-targeting crRNA molecule, and a tracrRNA molecule. Optionally, the lentivirus includes the nuclease protein variant, DNA-targeting crRNA molecule, and a tracrRNA molecule, and DNA donor template molecule for homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Agl4, HeLa, HEK293 (e.g., HEK293-F, HEK293-H, HEK293-T), and perC6 cells, any plant cell (differentiated or undifferentiated) as well as insect cells such as Spodoptera fugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with nuclease systems (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-.gamma. and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example see Inaba et al., J. Exp. Med. 176:1693-1702 (1992)). Stem cells that have been modified may also be used in some embodiments.

Notably, any one of the OMNI-50 variant described herein may be suitable for genome editing in post-mitotic cells or any cell which is not actively dividing, e.g., arrested cells. Examples of post-mitotic cells which may be edited using an OMNI-50 variant of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17^{th} ed., 1989).

### DNA Repair by Homologous Recombination

In some embodiments of the present invention, a variant OMNI-50 nuclease is utilized to affect a DNA break at a target site to induce cellular repair mechanisms, for example, but not limited to, non-homologous end-joining (NHEJ) or homology-directed repair (HDR).

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double strand break in a gene prior to repair and (2) a donor RNA template for repair, and the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. Non-viral vectors, such as transposon-based systems, e.g. recombinant Sleeping Beauty transposon systems or recombinant PiggyBac transposon systems, may also be utilized for transposition of a polynucleotide sequence in a target cell.

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

### Dna-Targeting RNA Molecules

In embodiments of the present invention, the DNA-targeting RNA sequence comprises a guide sequence portion. The “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, 25, 26, 27, 28, 29 or 30 nucleotides in length, or approximately 17-30, 17-29, 17-28, 17-27, 17-26, 27-25, 17-24, 18-22, 19-22, 18-20, 17-20, or 21-22 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, the RNA molecule is capable of targeting the CRISPR nuclease to the specific target DNA sequence. Each possibility represents a separate embodiment. An RNA molecule can be custom designed to target any desired sequence.

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell. In some embodiments, genome modifying occurs within the nucleus of a cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, beta thalassemia, sickle cell anemia, cell dysregulation and oncology diseases and disorders, inflammation and immune-related diseases and disorders, metabolic, liver, hypercholesteremia, kidney and protein diseases and disorders, muscular and skeletal diseases and disorders, dermatological diseases and disorders, neurological and neuronal diseases and disorders, pulmonary disease and disorders, corneal disease and disorders, retinal diseases and disorders, and ocular diseases and disorders.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nuclease disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-50 nuclease to a target site is designed to contain a spacer sequence complementary to a DNA strand of a DNA double-stranded region that neighbors a OMNI-140 PAM sequence, e.g. “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia. Guide RNA molecules which target the ELANE gene and are useful for treating neutropenia are disclosed in PCT International Application No. PCT/US2020/059186, incorporated herein by reference.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/ | |
|---|---|
| DISORDERS | GENE(S) |
| Neoplasia | PTEN; ATM; ATR; EGFR; ERBB2; ERBB3; ERBB4; |
| | Notch1; Notch2; Notch3; Notch4; AKT; AKT2; AKT3; |
| | HIF; HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); gf2 (3 variants); Igf 1 Receptor; Igf 2 Receptor; |
| | Bax; Bcl2; caspases family (9 members: 1, 2, 3, 4, 6, 7, 8, |
| | 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; |
| | GSK3b |
| Neurological, Neuro- | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and |
| | Atn1 (DRPLA Dx); CBP (Creb-BP - global instability); |
| | VLDLR (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion related disorders | Prp |
| ALS | SOD1; ALS2; STEX; FUS; TARDBP; VEGF (VEGF-a; |
| | VEGF-b; VEGF-c) |
| Addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile |
| | X (FMR2 (AFF2); FXR1; FXR2; Mglur5) |
| Alzheimer's Disease | E1; CHIP; UCH; UBB; Tau; LRP; PICALM; Clusterin; |
| | PS1; SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); |
| | IL-17b; IL-17c; IL-17d; IL-17f); II-23; Cx3cr1; ptpn22; |
| | TNFa; NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, |
| diseases and disorders | NT5C3, UMPH1, PSN1, RHAG, RH50A, NRAMP2, |
| | SPTB, ALAS2, ANH1, ASB, ABCB7, ABC7, ASAT); |
| | Bare lymphocyte syndrome (TAPBP, TPSN, TAP2, |
| | ABCB3, PSF2, RING11, MHC2TA, C2TA, RFX5, |
| | RFXAP, RFX5), Bleeding disorders (TBXA2R, P2RX1, |
| | P2X1); Factor H and factor H-like 1 (HF1, CFH, HUS); |
| | Factor V and factor VIII (MCFD2); Factor VII deficiency |
| | (F7); Factor X deficiency (F10); Factor XI deficiency |
| | (F11); Factor XII deficiency (F12, HAF); Factor XIIIA |
| | deficiency (F13A1, F13A); Factor XIIIB deficiency |
| | (F13B); Fanconi anemia (FANCA, FACA, FA1, FA, FAA, |
| | FAAP95, FAAP90, FLJ34064, FANCB, FANCC, FACC, |
| | BRCA2, FANCD1, FANCD2, FANCD, FACD, FAD, |
| | FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, |
| | HPLH2, UNC13D, MUNC13-4, HPLH3, HLH3, FHL3); |
| | Hemophilia A (F8, F8C, HEMA); Hemophilia B (F9, |
| | HEMB), Hemorrhagic disorders (PI, ATT, F5); Leukocyde |
| | deficiencies and disorders (ITGB2, CD18, LCAMB, LAD, |
| | EIF2B1, EIF2BA, EIF2B2, EIF2B3, EIF2B5, LVWM, |
| | CACH, CLE, EIF2B4); Sickle cell anemia (HBB); |
| | Thalassemia (HBA2, HBB, HBD, LCRB, HBA1) |
| Cell dysregulation and | B-cell non-Hodgkin lymphoma (BCL7A, BCL7); |
| oncology diseases and | Leukemia (TAL1, TCL5, SCL, TAL2, FLT3, NBS1, NBS, |
| disorders | ZNFN1A1, IK1, LYF1, HOXD4, HOX4B, BCR, CML, |
| | PHL, ALL, ARNT, KRAS2, RASK2, GMPS, AF10, |
| | ARHGEF12, LARG, KIAA0382, CALM, CLTH, CEBPA, |
| | CEBP, CHIC2, BTL, FLT3, KIT, PBT, LPP, NPM1, |
| | NUP214, D9S46E, CAN, CAIN, RUNX1, CBFA2, AML1, |
| | WHSC1L1, NSD3, FLT3, AF1Q, NPM1, NUMA1, |
| | ZNF145, PLZF, PML, MYL, STAT5B, AF10, CALM, |
| | CLTH, ARL11, ARLTS1, P2RX7, P2X7, BCR, CML, |
| | PHL, ALL, GRAF, NF1, VRNF, WSS, NENS, PTPN11, |
| | PTP2C, SHP2, NS1, BCL2, CCND1, PRAD1, BCL1, |
| | TCRA, GATA1, GF1, ERYF1, NFE1, ABL1, NQO1, |
| | DIA4, NMOR1, NUP214, D9S46E, CAN, CAIN) |
| Inflammation and | AIDS (KIR3DL1, NKAT3, NKB1, AMB11, KIR3DS1, |
| immune related diseases | IFNG, CXCL12, SDF1); Autoimmune lymphoproliferative |
| and disorders | syndrome (TNFRSF6, APT1, FAS, CD95, ALPS1A); |
| | Combined immunodeficiency, (IL2RG, SCIDX1, SCIDX, |
| | IMD4); HIV-1 (CCL5, SCYA5, D17S136E, TCP228), HIV |
| | susceptibility or infection (IL10, CSIF, CMKBR2, CCR2, |
| | CMKBR5, CCCKR5 (CCR5)); Immunodeficiencies |
| | (CD3E, CD3G, AICDA, AID, HIGM2, TNFRSF5, CD40, |
| | UNG, DGU, HIGM4, TNFSF5, CD40LG, HIGM1, IGM, |
| | FOXP3, IPEX, AIID, XPID, PIDX, TNFRSF14B, TACI); |
| | Inflammation (IL-10, IL-1 (IL-1a, IL-1b), IL-13, IL-17 |
| | (IL-17a (CTLA8), IL-17b, IL-17c, IL- 17d, IL-17f), II-23, |
| | Cx3cr1, ptpn22, TNFa, NOD2/CARD15 for IBD, IL-6, IL-12 |
| | (IL-12a, IL-12b), CTLA4, Cx3cl1); Severe combined |
| | immunodeficiencies (SCIDs)(JAK3, JAKL, DCLRE1C, |
| | ARTEMIS, SCIDA, RAG1, RAG2, ADA, PTPRC, CD45, |
| | LCA, IL7R, CD3D, T3D, IL2RG, SCIDX1, SCIDX, IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, |
| | G6PT, G6PT1, GAA, LAMP2, LAMPB, AGL, GDE, |
| | GBE1, GYS2, PYGL, PFKM); Hepatic adenoma, 142330 |
| | (TCF1, HNF1A, MODY3), Hepatic failure, early onset, |
| | and neurologic disorder (SCOD1, SCO1), Hepatic lipase |
| | deficiency (LIPC), Hepatoblastoma, cancer and carcinomas |
| | (CTNNB1, PDGFRL, PDGRL, PRLTS, AXIN1, AXIN, |
| | CTNNB1, TP53, P53, LFS1, IGF2R, MPRI, MET, CASP8, |
| | MCH5; Medullary cystic kidney disease (UMOD, HNFJ, |
| | FJHN, MCKD2, ADMCKD2); Phenylketonuria (PAH, |
| | PKU1, QDPR, DHPR, PTS); Polycystic kidney and hepatic |
| | disease (FCYT, PKHD1, ARPKD, PKD1, PKD2, PKD4, |
| | PKDTS, PRKCSH, G19P1, PCLD, SEC63) |
| Muscular/Skeletal | Becker muscular dystrophy (DMD, BMD, MYF6), |
| diseases and disorders | Duchenne Muscular Dystrophy (DMD, BMD); Emery- |
| | Dreifuss muscular dystrophy (LMNA, LMN1, EMD2, |
| | FPLD, CMD1A, HGPS, LGMD1B, LMNA, LMN1, |
| | EMD2, FPLD, CMD1A); Facioscapulohumeral muscular |
| | dystrophy (FSHMD1A, FSHD1A); Muscular dystrophy |
| | (FKRP, MDC1C, LGMD2I, LAMA2, LAMM, LARGE, |
| | KIAA0609, MDC1D, FCMD, TTID, MYOT, CAPN3, |
| | CANP3, DYSF, LGMD2B, SGCG, LGMD2C, DMDA1, |
| | SCG3, SGCA, ADL, DAG2, LGMD2D, DMDA2, SGCB, |
| | LGMD2E, SGCD, SGD, LGMD2F, CMD1L, TCAP, |
| | LGMD2G, CMD1N, TRIM32, HT2A, LGMD2H, FKRP, |
| | MDC1C, LGMD2I, TTN, CMD1G, TMD, LGMD2J, |
| | POMT1, CAV3, LGMD1C, SEPN1, SELN, RSMD1, |
| | PLEC1, PLTN, EBS1); Osteopetrosis (LRP5, BMND1, |
| | LRP7, LR3, OPPG, VBCH2, CLCN7, CLC7, OPTA2, |
| | OSTM1, GL, TCIRG1, TIRC7, OC116, OPTB1); Muscular |
| | atrophy (VAPB, VAPC, ALS8, SMN1, SMA1, SMA2, |
| | SMA3, SMA4, BSCL2, SPG17, GARS, SMAD1, CMT2D, |
| | HEXB, IGHMBP2, SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), |
| | Ehlers-Danlos syndrome (COL5A1, COL5A2, COL1A1, |
| | COL1A2, COL3A1, TNXB, ADAMTS2, PLOD1, |
| | FKBP14), Ichthyosis-associated disorders (FLG, STS, |
| | TGM1, ALOXE3/ALOX12B, KRT1, KRT10, ABCA12, |
| | KRT2, GJB2, TGM1, ABCA12, CYP4F22, ALOXE3, |
| | CERS3, NSHDL, EBP, MBTPS2, GJB2, SPINK5, |
| | AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging |
| | syndromes (POLR3A, PYCR1, LMNA, POLD1, WRN, DMPK) |
| Neurological and | ALS (SOD1, ALS2, STEX, FUS, TARDBP, VEGF |
| Neuronal diseases and | (VEGF-a, VEGF-b, VEGF-c); Alzheimer disease (APP, |
| disorders | AAA, CVAP, AD1, APOE, AD2, PSEN2, AD4, STM2, |
| | APBB2, FE65L1, NOS3, PLAU, URK, ACE, DCP1, |
| | ACE1, MPO, PACIP1, PAXIP1L, PTIP, A2M, BLMH, |
| | BMH, PSEN1, AD3); Autism (Mecp2, BZRAP1, MDGA2, |
| | Sema5A, Neurexin 1, GLO1, MECP2, RTT, PPMX, |
| | MRX16, MRX79, NLGN3, NLGN4, KIAA1260, |
| | AUTSX2); Fragile X Syndrome (FMR2, FXR1, FXR2, |
| | mGLUR5); Huntington's disease and disease like disorders |
| | (HD, IT15, PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, PARK4, |
| | DJ1, PARK7, LRRK2, PARK8, PINK1, PARK6, UCHL1, |
| | PARK5, SNCA, NACP, PARK1, PARK4, PRKN, PARK2, |
| | PDJ, DBH, NDUFV2); Rett syndrome (MECP2, RTT, |
| | PPMX, MRX16, MRX79, CDKL5, STK9, MECP2, RTT, |
| | PPMX, MRX16, |
| | MRX79, x-Synuclein, DJ-1); Schizophrenia (Neuregulin1 |
| | (Nrg1), Erb4 (receptor for Neuregulin), Complexin1 |
| | (Cplx1), Tph1 Tryptophan hydroxylase, Tph2, Tryptophan |
| | hydroxylase 2, Neurexin 1, GSK3, GSK3a, GSK3b, 5-HTT |
| | (Slc6a4), COMT, DRD (Drd1a), SLC6A3, DAOA, |
| | DTNBP1, Dao (Dao1)); Secretase Related Disorders |
| | (APH-1 (alpha and beta), Presenilin (Psen1), nicastrin, |
| | (Ncstn), PEN-2, Nos1, Parp1, Natl, Nat2); Trinucleotide |
| | Repeat Disorders (HTT (Huntington's Dx), |
| | SBMA/SMAX1/AR (Kennedy's Dx), FXN/X25 |
| | (Friedrich's Ataxia), ATX3 (Machado-Joseph's Dx), |
| | ATXN1 and ATXN2 (spinocerebellar ataxias), DMPK |
| | (myotonic dystrophy), Atrophin-1 and Atn1 (DRPLA Dx), |
| | CBP (Creb-BP - global instability), VLDLR (Alzheimer's), |
| | Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, |
| | CP49, CP47, CRYAA, CRYA1, PAX6, AN2, MGDA, |
| | CRYBA1, CRYB1, CRYGC, CRYG3, CCL, LIM2, MP19, |
| | CRYGD, CRYG4, BFSP2, CP49, CP47, HSF4, CTM, |
| | HSF4, CTM, MIP, AQP0, CRYAB, CRYA2, CTPP2, |
| | CRYBB1, CRYGD, CRYG4, CRYBB2, CRYB2, CRYGC, |
| | CRYG3, CCL, CRYAA, CRYA1, GJA8, CX50, CAE1, |
| | GJA3, CX46, CZP3, CAE3, CCM1, CAM, KRIT1); |
| | Corneal clouding and dystrophy (APOA1, TGFBI, CSD2, |
| | CDGG1, CSD, BIGH3, CDG2, TACSTD2, TROP2, MIS1, |
| | VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, |
| | GPOA, OPTN, GLC1E, FIP2, HYPL, NRP, CYP1B1, |
| | GLC3A, OPA1, NTG, NPG, CYP1B1, GLC3A); Leber congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular |
| | dystrophy (ELOVL4, ADMD, STGD2, STGD3, RDS, |
| | RP7, PRPH2, PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease with the targeting sequence serving as the targeting portion of the CRISPR complex. When the molecule having the targeting sequence is present contemporaneously with the CRISPR molecule the RNA molecule is capable of targeting the CRISPR nuclease to the specific target sequence. Each possibility represents a separate embodiment. An RNA molecule can be custom designed to target any desired sequence.

The term “targets” as used herein, refers to a targeting sequence or targeting molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

As used herein the term “wild-type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild-type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion), also referred to as a “mutation,” compared to the wild-type OMNI-50 nuclease of SEQ ID NO:1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

The terms “mutant” or “variant” are used interchangeably and indicate a molecule that is non-naturally occurring or engineered.

As used herein the term “amino acid” includes natural and/or unnatural or synthetic amino acids, including glycine and both the D- or L-, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

As used herein, the term “modified cells” refers to cells in which a double strand break is affected by a complex of an RNA molecule and the CRISPR nuclease variant as a result of hybridization with the target sequence, i.e. on-target hybridization. The term “modified cells” may further encompass cells in which a repair or correction of a mutation was affected following the double strand break induced by the variant. The modified cell may be any type of cell e.g., eukaryotic or prokaryotic, in any environment e.g., isolated or not, maintained in culture, in vitro, ex vivo, in vivo or in planta.

This invention provides a modified cell or cells obtained by use of any of the variants or methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cell (HSC), or any cell suitable for an allogenic cell transplant or autologous cell transplant. The variants and methods described herein may also be utilized to generate chimeric antigen receptor T (CAR-T) cells.

This invention also provides a composition comprising these modified cells and a pharmaceutically acceptable carrier. Also provided is an in vitro or ex vivo method of preparing this, comprising mixing the cells with the pharmaceutically acceptable carrier.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The terms “protospacer adjacent motif” or “PAM” as used herein refers to a nucleotide sequence of a target DNA located in proximity to the targeted DNA sequence and recognized by the CRISPR nuclease. The PAM sequence may differ depending on the nuclease identity. For example, wild-type Streptococcus pyogenes Cas9 recognizes a “NGG” PAM sequence. A skilled artisan will appreciate that single-guide RNA molecules or crRNA:tracrRNA complexes capable of complexing with a CRISPR nuclease such as to associate with a target genomic DNA sequence of interest next to a protospacer adjacent motif (PAM). The nuclease then mediates cleavage of target DNA to create a double-stranded break within the protospacer.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPAl M9 NLS, the IBB domain from importin-alpha, myoma T protein, human p53, mouse c-abl IV, influenza vims NS1, Hepatitis virus delta antigen, mouse Mx1 protein, human poly(ADP-ribose) polymerase, and the steroid hormone receptors (human) glucocorticoid.

The term “CRISPR system” refers to a CRISPR endonuclease system that includes a CRISPR nuclease protein, such as the mutants or variants described herein, and a suitable guide RNA molecule or guide RNA complex, e.g. a single-guide RNA or a crRNA:tracrRNA complex, for targeting the CRISPR nuclease protein to a desired target DNA sequence based on complementarity between a portion of the guide RNA molecule or guide RNA complex and the target DNA sequence. The term “wild-type CRISPR endonuclease system” refers to a CRISPR endonuclease system that includes wild-type CRISPR protein and a suitable guide RNA molecule or guide RNA complex, e.g. a single-guide RNA or a crRNA:tracrRNA complex, for targeting the wild-type CRISPR nuclease protein to a desired target DNA sequence based on complementarity between a portion of the guide RNA molecule or guide RNA complex and the target DNA sequence.

In the context of the invention, “maintained on-target editing activity” refers to the ability of an OMNI-50 variant to target a DNA target site that is targeted by a guide RNA molecule associated with, and thereby programming, the OMNI-50 variant. In some embodiments, the OMNI-50 variant maintains on-target editing activity of a DNA target at a percent editing level greater than or equal to the percent editing level of a wild-type OMNI-50 nuclease for the DNA target. In some embodiments, the OMNI-50 variant maintains on-target editing activity of a DNA target of at least 100%, 95%, 90%, 80%, 70%, 60%, 50%, 40%, or 30% the level of percent editing of a wild-type OMNI-50 nuclease for the DNA target.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells—A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1: Variant Selection

In order to select OMNI-50 nuclease variants with increased specificity to the target site (e.g. increased ratio between On-target cuts and Off-target cuts), amino acid substitutions were introduced into the open reading frame of the wild-type OMNI-50 sequence (SEQ ID NO: 1) as shown in Table 1.

TABLE 1
Summary of amino acid positions wild-type OMNI-50 nuclease and variants thereof. A blank box indicates the
OMNI-50 variant has the same amino acid as the wild-type OMNI-50 nuclease in the indicated position.

| | Substitutions | | | | | | | | | | | | | | | | | |
|---|---|---|---|---|---|---|---|---|---|---|---|---|---|---|---|---|---|---|
| Variant | relative to | Amino acid identity by position | | | | | | | | | | | | | | | | |
| name | WT OMNI-50 | 61 | 437 | 478 | 493 | 545 | 606 | 688 | 690 | 695 | 718 | 788 | 803 | 805 | 844 | 965 | 981 | 1036 |
| WT | Not | R | Y | R | A | Y | G | K | L | E | L | R | Q | L | L | K | V | K |
| OMNI-50 | applicable | | | | | | | | | | | | | | | | | |
| Variant | R478I; | | | I | | H | | | | | | | V | I | | | | |
| 3795 | Y545H; | | | | | | | | | | | | | | | | | |
| | Q803V; | | | | | | | | | | | | | | | | | |
| | L805I | | | | | | | | | | | | | | | | | |
| Variant | R61A; | A | W | | | | | | | | | K | | | N | | M | |
| 3942 | Y437W; | | | | | | | | | | | | | | | | | |
| | R788K; | | | | | | | | | | | | | | | | | |
| | L844N; | | | | | | | | | | | | | | | | | |
| | V981M | | | | | | | | | | | | | | | | | |
| Variant | G606P; | | | | | | P | | V | V | | | | | | | | |
| 2902 | L690V; | | | | | | | | | | | | | | | | | |
| | E695V | | | | | | | | | | | | | | | | | |
| Variant | R478T; | | | T | G | | | | | | C | | | | | V | | V |
| 3954 | A493G; | | | | | | | | | | | | | | | | | |
| | L718C; | | | | | | | | | | | | | | | | | |
| | K965V; | | | | | | | | | | | | | | | | | |
| | K1036V | | | | | | | | | | | | | | | | | |
| Variant | Q803V | | | | | | | | | | | | V | | | | | |
| 6918 | | | | | | | | | | | | | | | | | | |
| Variant | Y545H | | | | | H | | | | | | | | | | | | |
| 6919 | | | | | | | | | | | | | | | | | | |
| Variant | L805I | | | | | | | | | | | | | I | | | | |
| 6920 | | | | | | | | | | | | | | | | | | |
| Variant | R478I; | | | I | | H | | | | | | | V | | | | | |
| 6864 | Y545H; | | | | | | | | | | | | | | | | | |
| | Q803V | | | | | | | | | | | | | | | | | |
| Variant | R478I; | | | I | | H | | | | | | | | | | | | |
| 6865 | Y545H | | | | | | | | | | | | | | | | | |
| Variant | R478I; | | | I | | | | | | | | | V | I | | | | |
| 6866 | Q803V; | | | | | | | | | | | | | | | | | |
| | L805I | | | | | | | | | | | | | | | | | |
| Variant | Y545H; | | | | | H | | | | | | | V | I | | | | |
| 6867 | Q803V; | | | | | | | | | | | | | | | | | |
| | L805I | | | | | | | | | | | | | | | | | |
| Variant | R478H; | | | H | | H | | | | | | | V | I | | | | |
| 7085 | Y545H; | | | | | | | | | | | | | | | | | |
| | Q803V; | | | | | | | | | | | | | | | | | |
| | L805I | | | | | | | | | | | | | | | | | |
| Variant | R478V; | | | V | | H | | | | | | | V | I | | | | |
| 7086 | Y545H; | | | | | | | | | | | | | | | | | |
| | Q803V; | | | | | | | | | | | | | | | | | |
| | L805I | | | | | | | | | | | | | | | | | |
| Variant | R478D; | | | D | | H | | | | | | | V | I | | | | |
| 7087 | Y545H; | | | | | | | | | | | | | | | | | |
| | Q803V; | | | | | | | | | | | | | | | | | |
| | L805I | | | | | | | | | | | | | | | | | |
| Variant | R478S; | | | S | | H | | | | | | | V | I | | | | |
| 7088 | Y545H; | | | | | | | | | | | | | | | | | |
| | Q803V; | | | | | | | | | | | | | | | | | |
| | L805I | | | | | | | | | | | | | | | | | |
| Variant | R478I; | | | I | | C | | | | | | | V | I | | | | |
| 7090 | Y545C; | | | | | | | | | | | | | | | | | |
| | Q803V; | | | | | | | | | | | | | | | | | |
| | L805I | | | | | | | | | | | | | | | | | |
| Variant | R478I; | | | I | | D | | | | | | | V | I | | | | |
| 7091 | Y545D; | | | | | | | | | | | | | | | | | |
| | Q803V; | | | | | | | | | | | | | | | | | |
| | L805I | | | | | | | | | | | | | | | | | |
| Variant | R478I; | | | I | | T | | | | | | | V | I | | | | |
| 7092 | Y545T; | | | | | | | | | | | | | | | | | |
| | Q803V; | | | | | | | | | | | | | | | | | |
| | L805I | | | | | | | | | | | | | | | | | |
| Variant | R478I; | | | I | | F | | | | | | | V | I | | | | |
| 7093 | Y545F; | | | | | | | | | | | | | | | | | |
| | Q803V; | | | | | | | | | | | | | | | | | |
| | L805I | | | | | | | | | | | | | | | | | |
| Variant | R478I; | | | I | | A | | | | | | | V | I | | | | |
| 7094 | Y545A; | | | | | | | | | | | | | | | | | |
| | Q803V; | | | | | | | | | | | | | | | | | |
| | L805I | | | | | | | | | | | | | | | | | |

TABLE 2
Guides Designed for discriminating SNPs and
used for testing OMNI-50 nuclease
variant activity and allele-specific editing.

| Target SNP | gRNA | Guide Sequence | Location | Mechanism |
|---|---|---|---|---|
| rs1683564 | g62 | GUCAAGCCCCA | Downstream | Excision, allele |
| | REF | GAGGCCACA | to 3′UTR | knock out |
| | 20 nt | (SEQ ID NO: 29) | | |
| | g62 | GUCAAGCCCCA | | |
| | ALT | GAGGACACA | | |
| | 20 nt | (SEQ ID NO: 30) | | |
| | sgRNA- | GUGUCAAGCCC | | |
| | 564DS-ref | CAGAGGCCACA | | |
| | 22 nt | (SEQ ID NO: 31) | | |
| | sgRNA- | GUGUCAAGCCC | | |
| | 564DS-alt | CAGAGGACACA | | |
| | 22 nt | (SEQ ID NO: 32) | | |
| | g35 | AGUCCGGGCUG | Intron 4 | Excision, allele |
| | 20 nt | GGAGCGGGU | | knock out |
| | | (SEQ ID NO: 33) | | |
| | sgRNA- | GCAGUCCGGGC | | |
| | Constant | UGGGAGCGGGU | | |
| | 22 nt | (SEQ ID NO: 34) | | |

### Example 2: OMNI-50 Variants Depict Allele Specific Editing of ELANE Down-Stream Composition

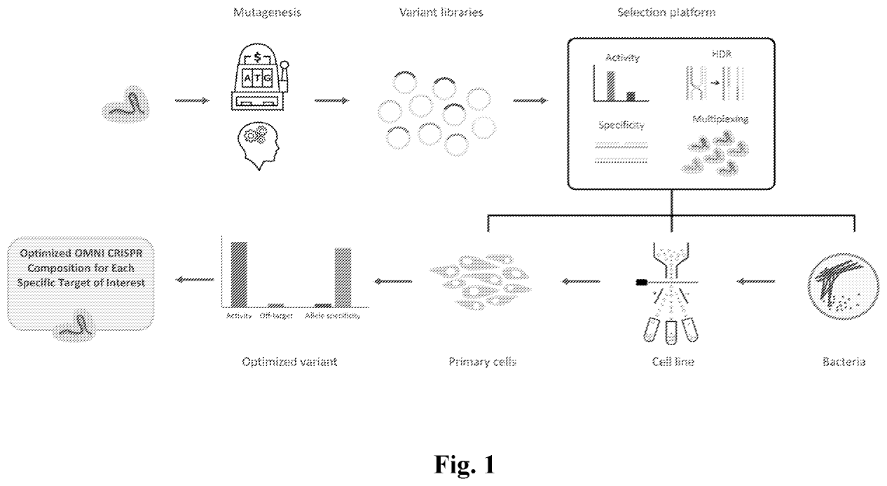

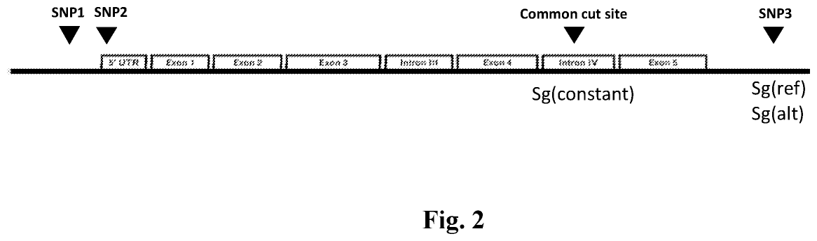

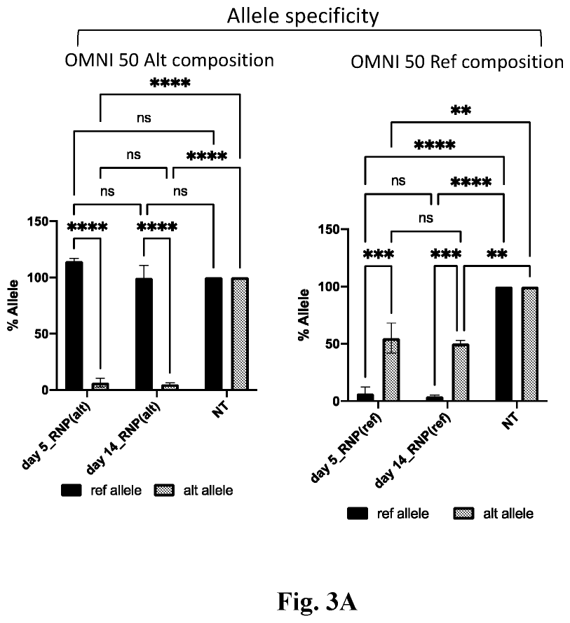

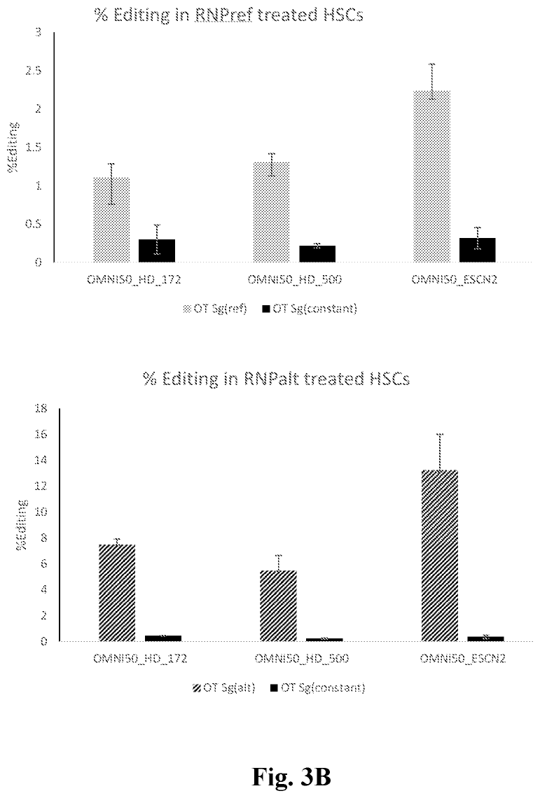

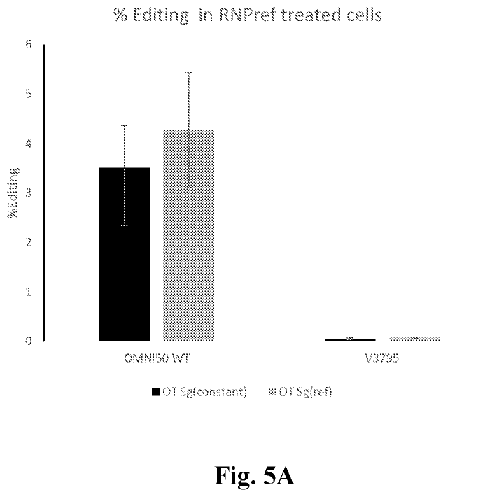

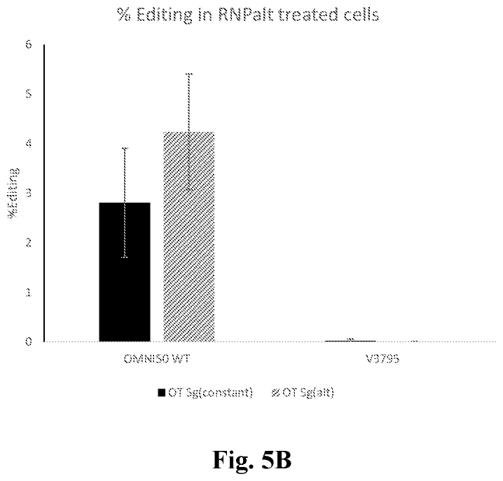

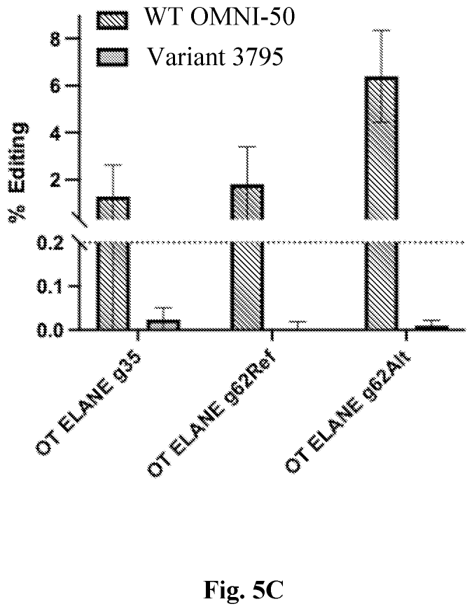

Improved specificity of OMNI-50 variant nucleases relative to wild-type OMNI-50 nuclease was demonstrated by examining the ability of the nucleases to discriminate between the Alternative and Reference sequences of rs1683564 by utilizing g62alt and g62ref RNA guide sequences. See FIGS. 1-6C.

While editing with sgRNA-564DS-alt depicted high degree of specificity with wild-type OMNI-50 nuclease, sgRNA-564DS-ref showed lack of discrimination represented by the editing of both reference and alternative allele. Allele specificity was tested by excision with four OMNI-50 nuclease variants v3942, v2902, v3954 and v3795 in HSCs.

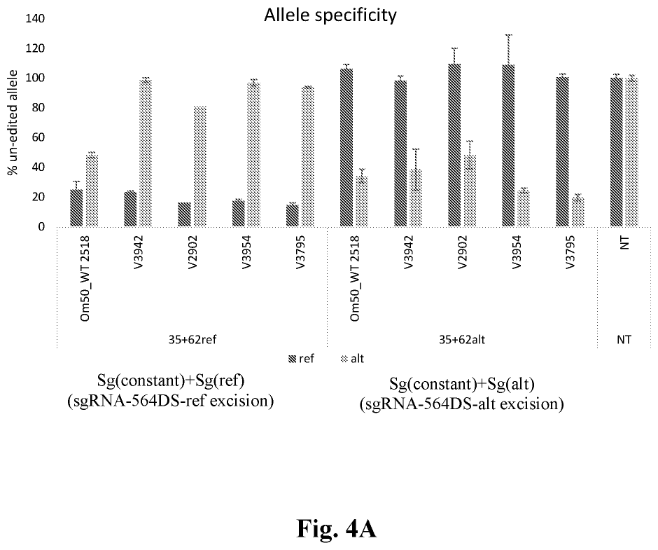

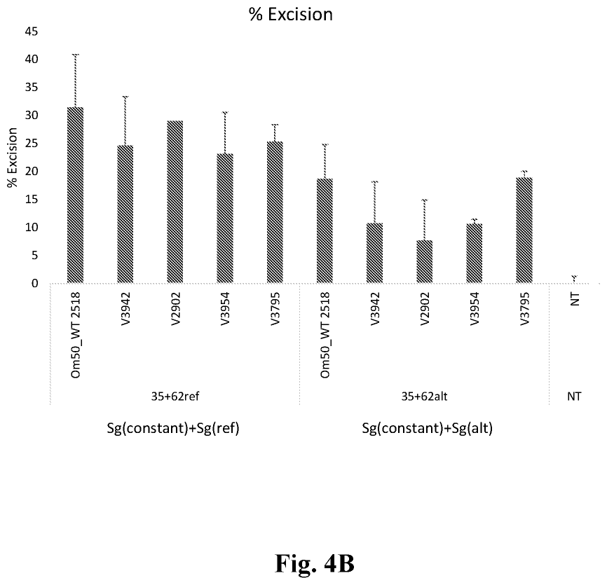

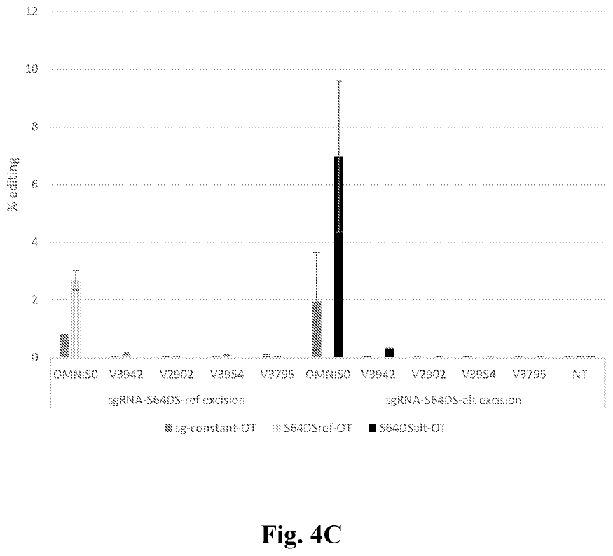

All variants of OMNI-50 revealed improved specificity for sgRNA-564DS-ref in comparison to OMNI-50 while maintaining sgRNA-564DS-alt allele discrimination determined by ddPCR (FIG. 4A). In addition to allele discrimination, one off-target to sgRNA-564DS-ref, sgRNA-564DS-alt and sg-constant were validated and showed high degree of variant fidelity in comparison to OMNI-50 characterized by <0.5% editing activity (FIG. 4B and FIG. 4C).

These results emphasize the relative high fidelity of OMNI-50 nuclease variants.

### Example 3: Mutant Allele Knock-Out with a Novel CRISPR Nuclease Promotes Myelopoiesis in ELANE Neutropenia

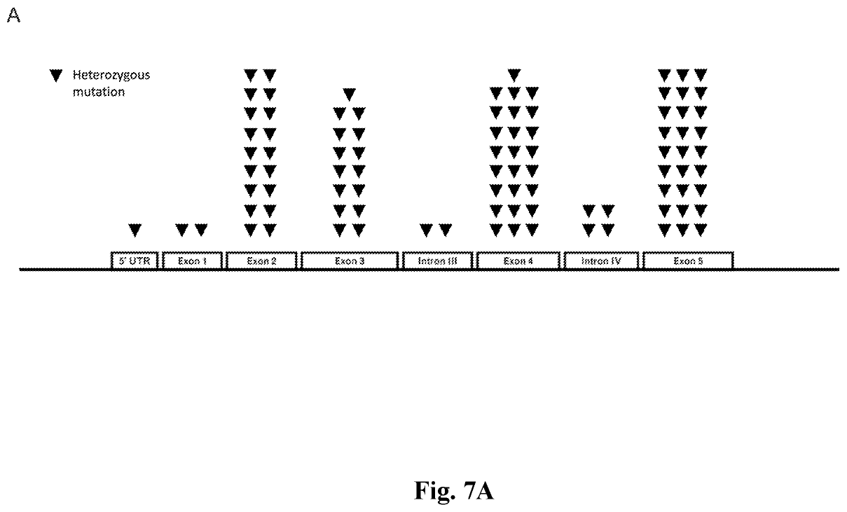

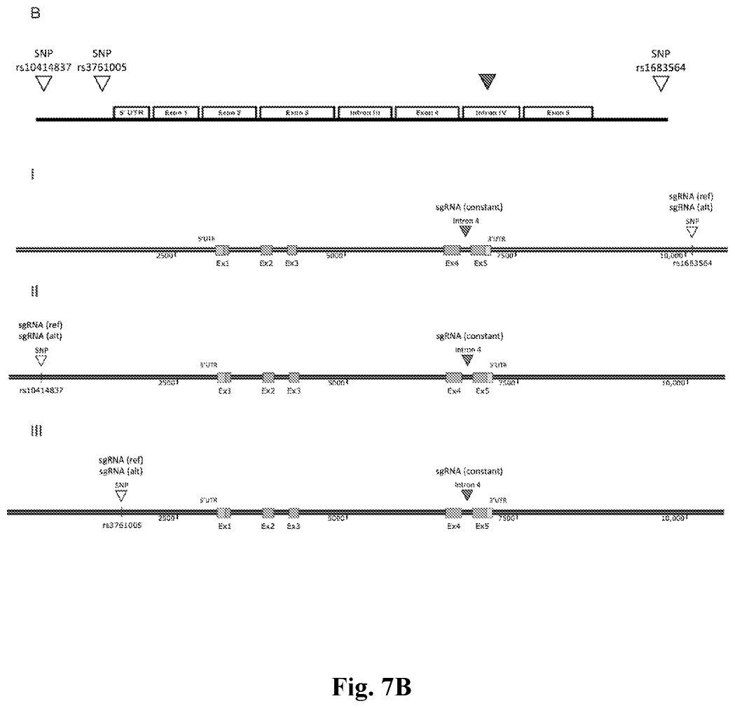

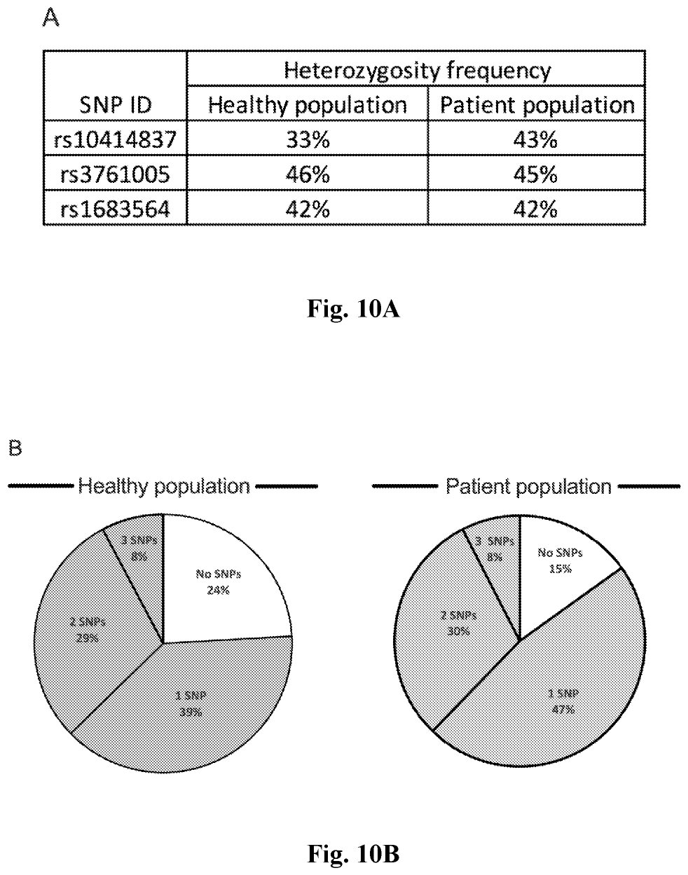

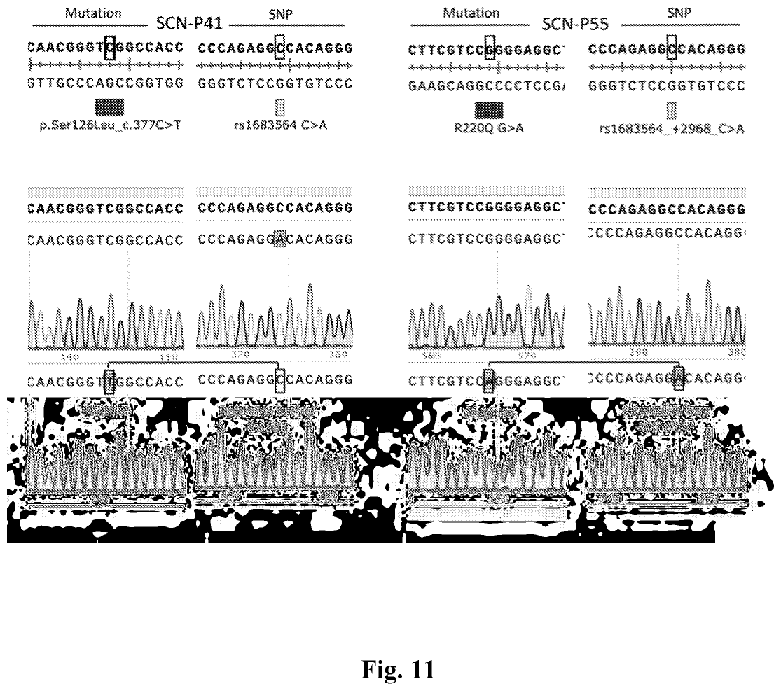

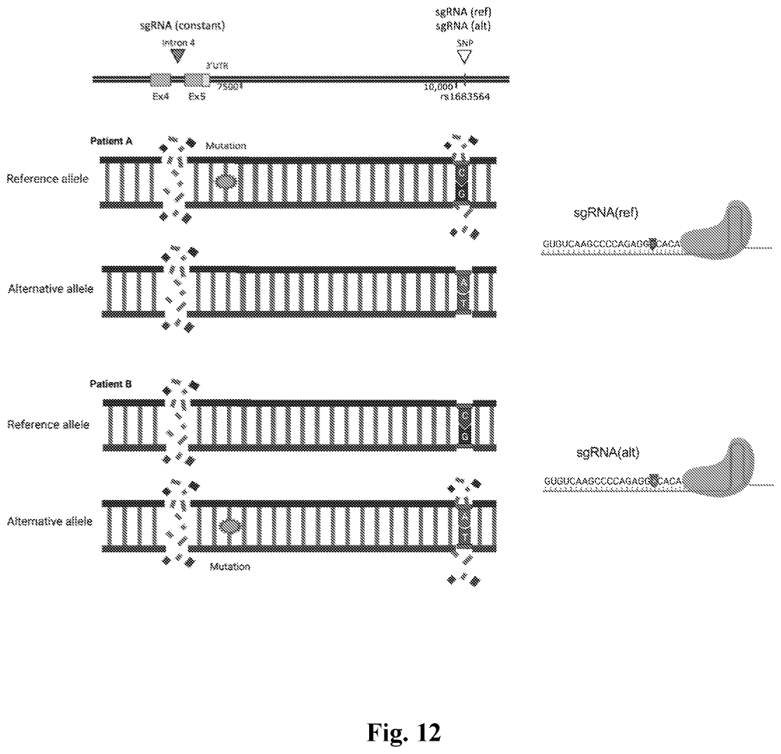

Representative ELANE mutations and respective therapeutic strategies. Severe congenital neutropenia (SCN) is associated with numerous heterozygous mutations in EL4NE gene (FIG. 7A). The current study presents a novel approach for removal of the mutated ELANE allele by targeting heterozygous sites of SNPs that are adjacent to the majority of ELANE-mediated SCN mutations, instead of independently editing each pathogenic mutation. Out of hundreds of potential SNPs in the ELANE gene, three SNPs retrieved from the healthy database 1000 Genomes Project Consortium were identified that are frequently heterozygous in the healthy population, termed herein: rs1683564, rs10414837 and rs3761005 (FIG. 7B upper panel and FIG. 10A). Analysis of the healthy and patient populations revealed similar heterozygosity frequency between the two populations in each of the SNPs. About 76-85% of each of the populations (healthy and patient, respectively) was heterozygous to at least one of the SNPs, indicating on the applicability of our strategy to more than about 75% of the SCN patient population. (FIG. 10B, Supplemental Table 1). For each of the three SNPs we developed an editing ribonucleoprotein (RNP) composition that includes two different guides and a novel optimized CRISPR/Cas9 nuclease termed OMNI Variant 3795. One guide (termed herein sgRNA constant) is common to all three compositions and cuts both ELANE alleles at intron 4. The second guide targets the heterozygous form of one of the three SNPs and therefore cuts only one allele. The composition which targets SNP rs1683564 excises exon 5 and the entire 3′ UTR, causing the degradation of the destabilized mRNA transcript (FIG. 7B, I). The compositions which target either SNP rs10414837 or SNP rs3761005 lead to excision of most of the coding region and the promotor, thus preventing the transcription of the mutated allele (FIG. 7B, II and III). The current study is focused on composition 1, targeting the rs1683564 SNP. The more prevalent form of rs1683564 SNP is cytosine and is referred to herein as reference (ref) allele. Adenosine is the less prevalence form of the SNP and is referred to herein as alternative (alt) allele. Prior to treatment, patient cells are genotyped to determine if the mutation and the SNP are on the same allele or different alleles in a process termed linkage determination (FIG. 11). If the pathogenic mutation is linked to the reference allele, a nuclease-guide RNA composition including a guide (sgRNA (ref)) that targets the cytosine form of the SNP is chosen (termed herein RNP(ref)). If the pathogenic mutation is linked to the alternative allele, a composition including a guide (sgRNA (alt)) that targets the adenosine form of the SNP is chosen (termed herein RNP(alt)). The guides differ by only one nucleotide and when used with the sgRNA constant result in the same editing outcome (FIG. 12).

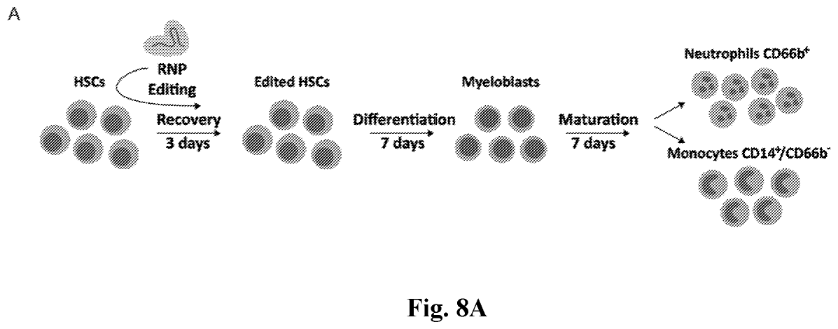

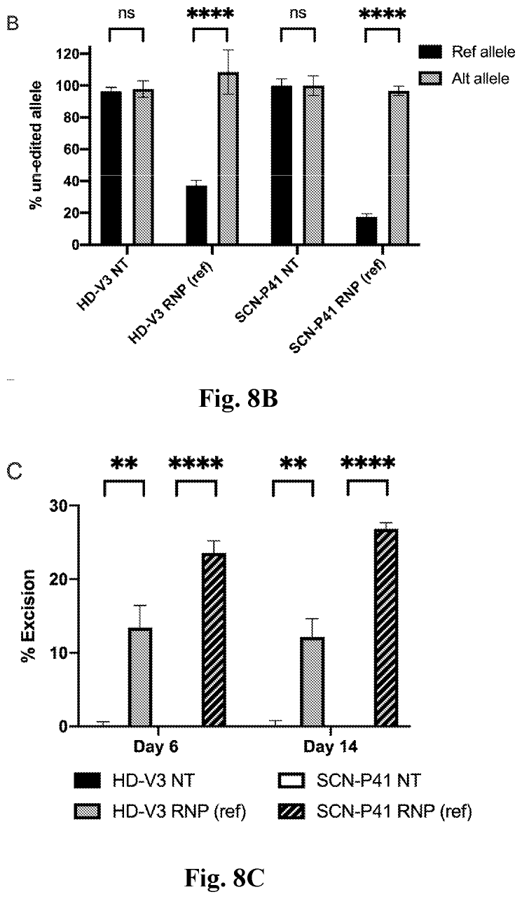

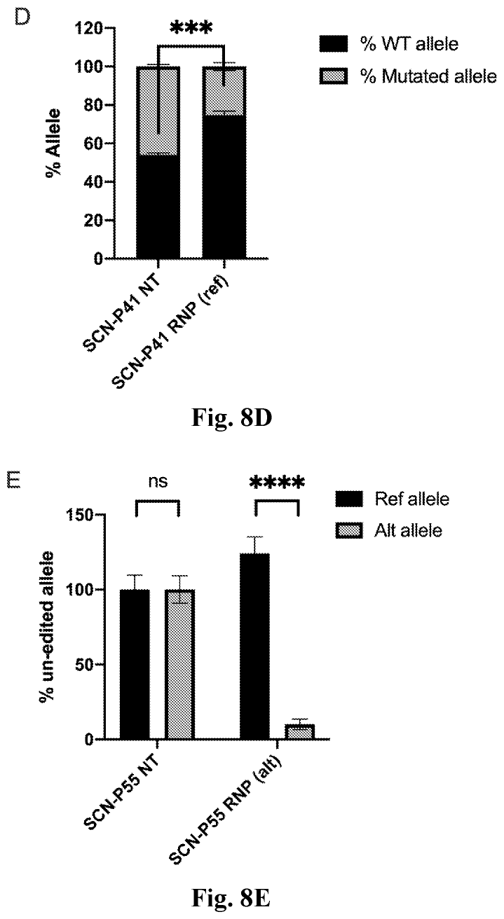

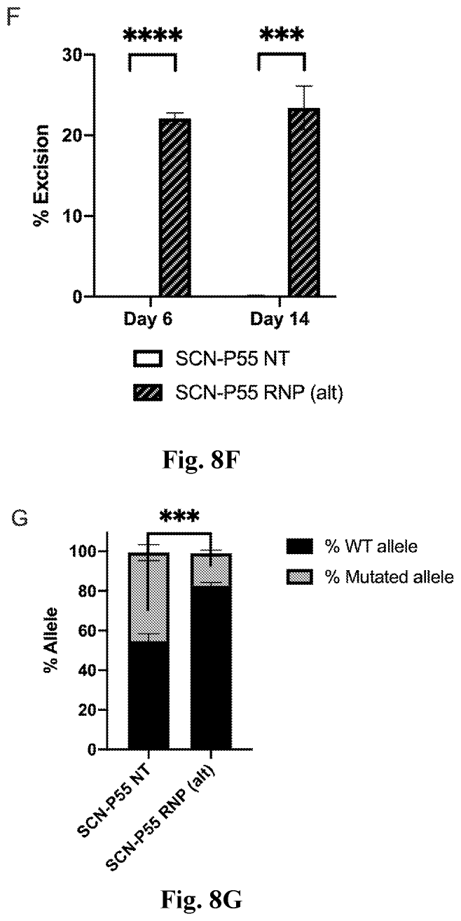

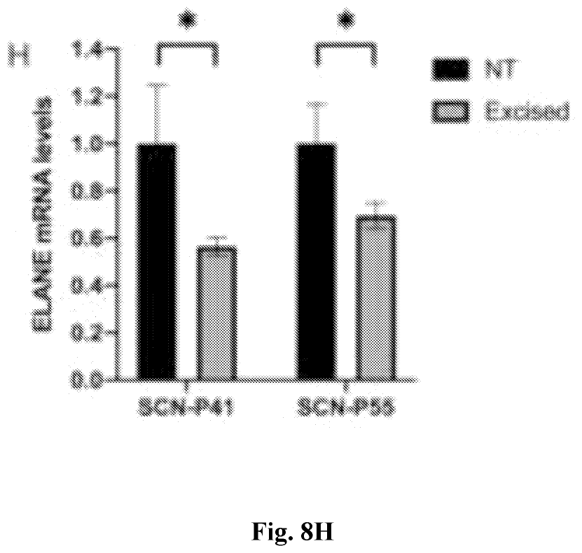

Allele specificity and excision efficiency using OMNI Variant 3795 nuclease in a SNP-based knock-out strategy. Unlike most CRISPR-associated editing strategies that cut the target gene in both alleles, our approach is directed at removal of the mutated allele while keeping the wild-type functional allele intact. To demonstrate the feasibility of our mono-allelic editing strategy, HSCs heterozygous to SNP rs1683564, taken from healthy donors and SCN patients were excised using either RNP(ref) or RNP(alt) (according to their linkage) or left non-treated (NT). HSCs recovered for 3 days in CD34⁺ expansion media, then cultured for 7 days in the presence of IL-3, SCF, GM-CSF and G-CSF for proliferation and myeloid progenitor differentiation, and subsequently stimulated with G-CSF for further 7 days for neutrophil differentiation (FIG. 8A).

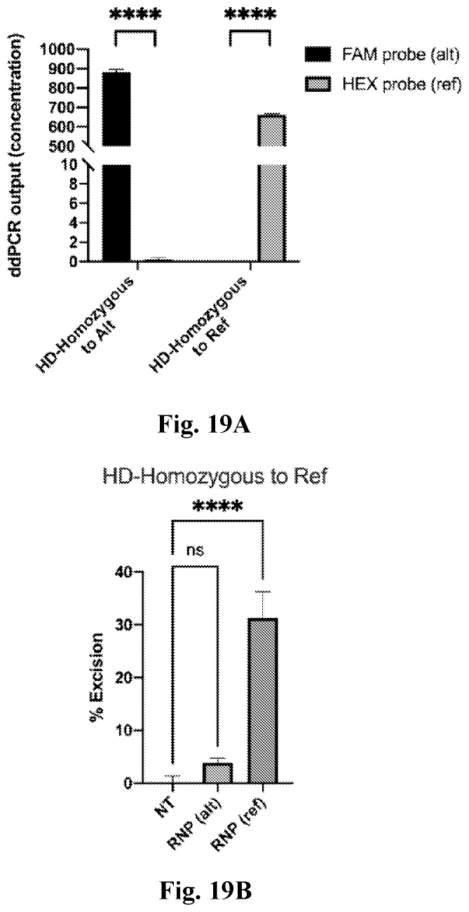

A fraction of cells was harvested at day 6 and 14 of differentiation for genomic DNA or RNA extraction. Allele specificity was determined by two competitive probes binding either the alternative allele or the reference allele. (For probes' specificity see Supplemental Methods and FIG. 19A and FIG. 19B). To test the RNP(ref) composition we used HSCs from P41 SCN patient (SCN-P41) harboring a mutation on the reference allele and HSCs from V3 healthy donor (HD-V3), both heterozygous to the reference form of the SNP. ddPCR revealed that editing using RNP(ref) was specific as only about 40% and about 20% of the reference allele remained intact in HSCs of HD-V3 and SCN-P41, respectively (FIG. 8B). The alternative allele, however, was not affected by the RNP(ref) composition as indicated by its high levels that were similar to those of non-treated cells (NT) (FIG. 8B). These results demonstrated that treatment with RNP(ref) composition leads to allele-specific editing.

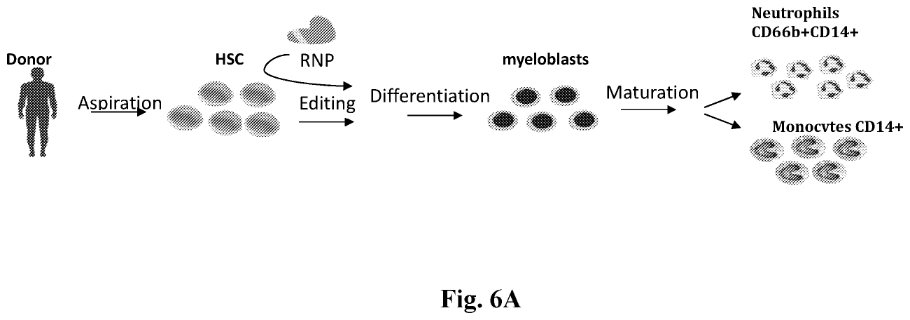

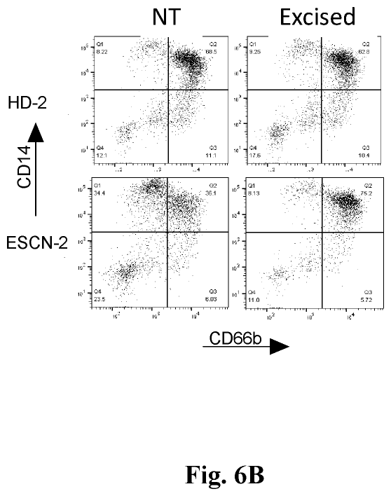

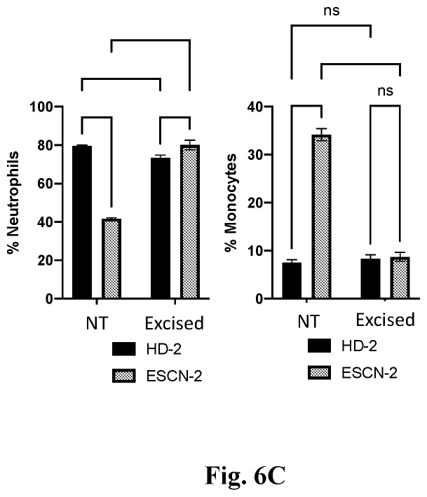

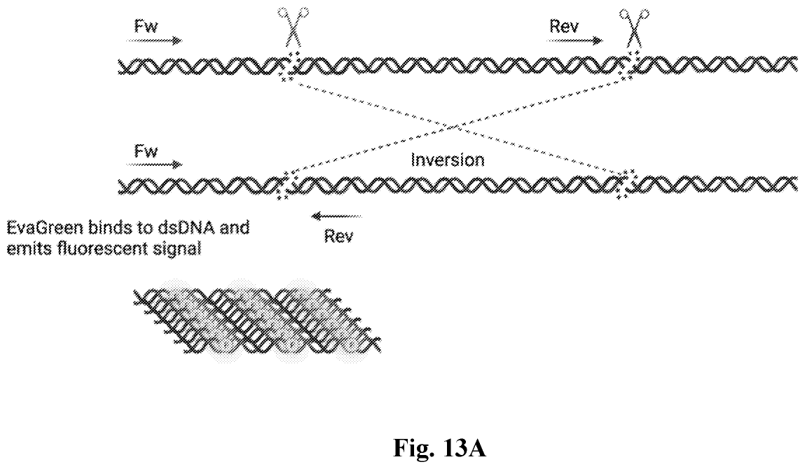

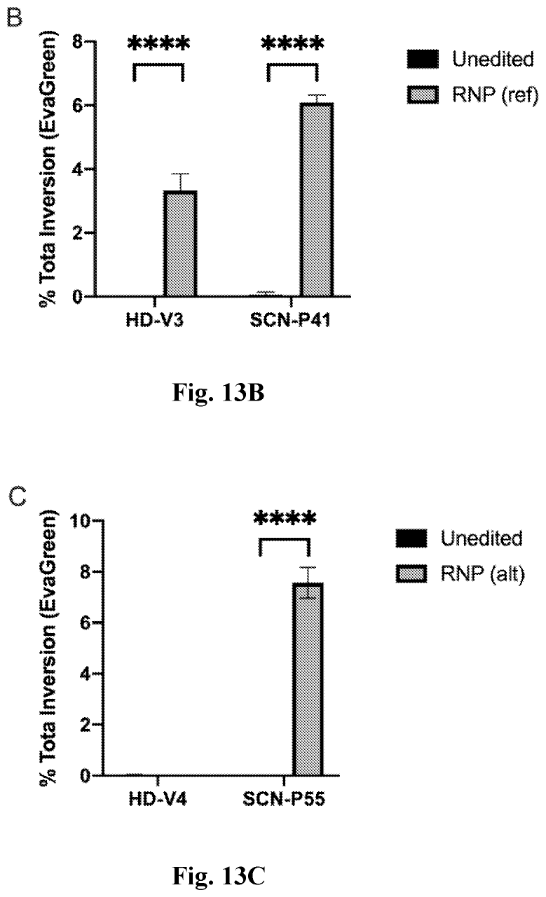

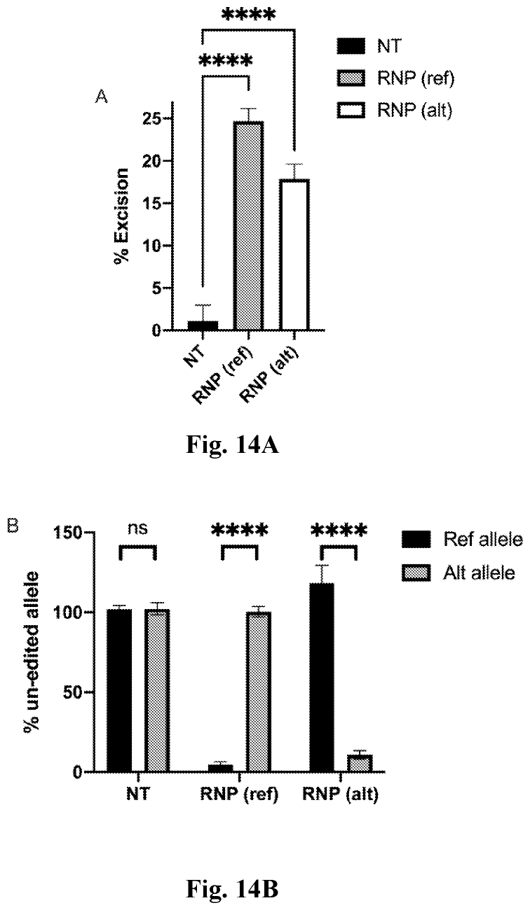

Next, we evaluated excision efficiency at day 6 and 14 of neutrophil differentiation. Excision was determined by amplification of two regions in ELANE gene using two differently labeled probes, one for exon 1 that is not affected by the current excision strategy and a second probe for exon 5 that is degraded upon excision (FIG. 6B, I). The ratio between the signals of the two probes was translated to excision efficiency. Treatment with RNP(ref) resulted in about 13% excision in HSCs of HD-V3 and about 25% excision in HSCs of SCN-P41 (FIG. 8C). Given the high specificity of the nuclease (FIG. 8B), this excision correlated to about 50% of the cell population that has undergone excision at the reference allele in HSCs of SCN-P41. RNP(ref) treatment also involved about 6% inversion events in HSCs of SCN-P41 as measured by EvaGreen staining (FIG. 13A and FIG. 13B). Of note, other experiments evaluating RNP(ref)-mediated excision levels in HSCs from healthy donors showed higher excision levels than reported above, that were comparable to those measured in HSCs of SCN-P41 (FIG. 14). In addition, NGS analysis of cDNA from SCN-P41 cells targeting exon 4-harboring mutation showed a 1:1 ratio between wild-type and mutated allele in non-treated cells. RNP(ref) treatment shifted the wild-type to mutated allele ratio to 3:1 by differentially reducing the mutated allele transcript, thereby enriching the wild-type allele (FIG. 8D).

To evaluate the specificity and efficiency of the RNP(alt) composition we used HSCs from P55 SCN patient (SCN-P55) harboring a mutation on the alternative allele and heterozygous to the alternative form of the SNP.

RNP(alt)-based treatment resulted in editing of about 90% of the alternative allele (only 10% remained intact), whereas the reference allele was kept intact at day 6 of differentiation (FIG. 8E). Excision levels in SCN-P55 HSCs were about 23% at day 6 and day 14 of neutrophil differentiation (FIG. 8F), indicating about 46% of the cell population has undergone excision at the alternative allele. RNP(alt) treatment resulted in 7.6% inversion events in SCN-P55 HSCs as measured by EvaGreen staining (FIG. 13C). Specificity and excision efficiency of RNP(alt)-edited HSCs of healthy donors was tested and found comparable to that obtained in patient-derived cells (FIG. 14). In addition, NGS analysis of cDNA from SCN-P55 cells targeting exon 5-harboring mutation showed enrichment of the wild-type allele (FIG. 8G). ELANE mRNA levels were decreased following excision in cells from SCN-P41 and SCN-P55 patients (FIG. 8H). In view of the increased WT:mutant allele ratio, obtained following excision (FIG. 8D and FIG. 8G), the reduced mRNA levels were mainly a result of the degradation of the mutated transcript.

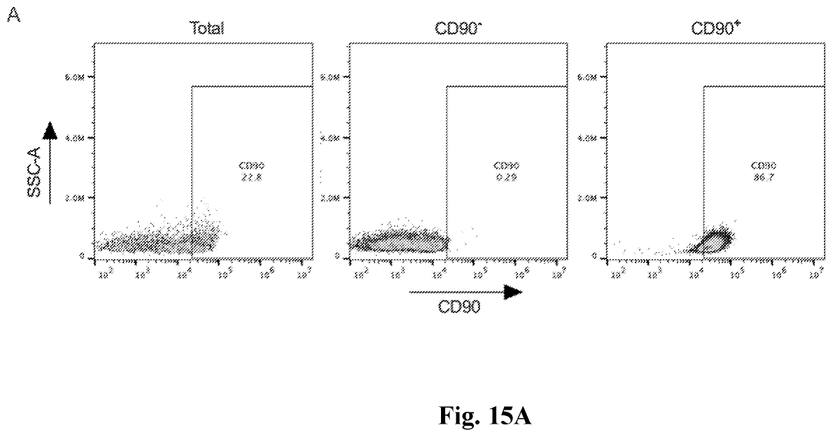

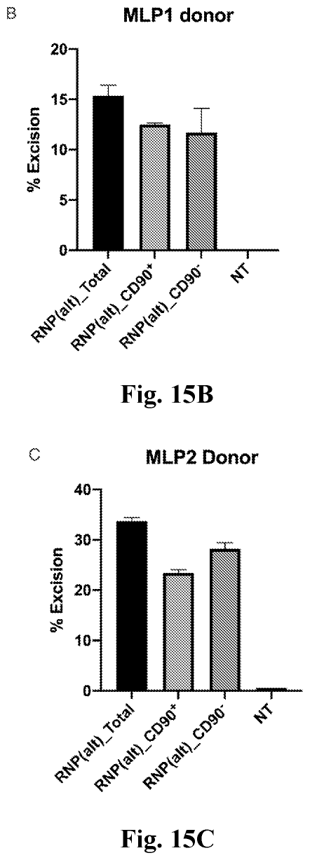

Next, we confirmed RNP(alt)-based excision has occurred in a sub-population of HSCs (CD34⁺/CD90⁺ cells) that is considered essential for multilineage engraftment and hematopoietic reconstitution (FIGS. 15A-15C).

HSCs from both P41 and P55, edited using RNP(ref) and RNP(alt), respectively, were analyzed by Amplicon NGS to measure editing on off targets identified for constant guide (SgRNA(constant)), reference guide (SgRNA(ref)) and alternative guide (SgRNA(alt)).

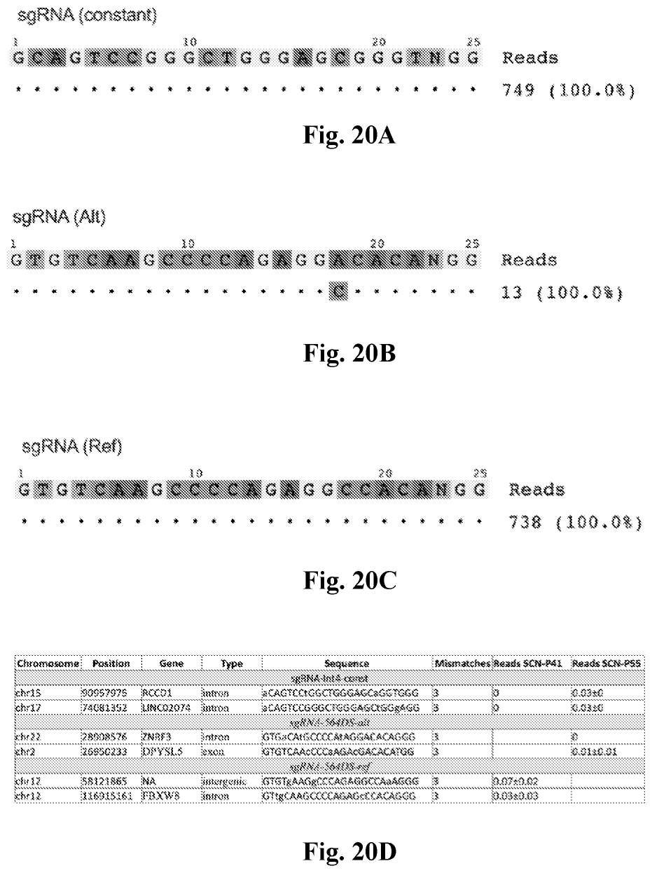

Specifically, an unbiased survey (GUIDE-seq) of whole-genome off-target cleavage using the Variant 3795 nuclease and each of the constant guide (SgRNA(constant)), reference guide (SgRNA(ref)) and alternative guide (SgRNA(alt)), resulted in no identified off-targets (≤4 mismatches) (FIGS. 20A-20C).

In addition, in-silico off-target analysis was performed for each of the guides and identified a few potential off-targets. None of these off-targets were validated by a rhAmpSeq analysis done on edited HSCs from patients SCN-P41 and SCN-P55 (FIG. 20D), demonstrating the high fidelity of the novel nuclease compositions.

Taken together, the results provided above present an active, highly accurate nuclease that can target the mutant allele while preserving the wild-type functional allele intact.

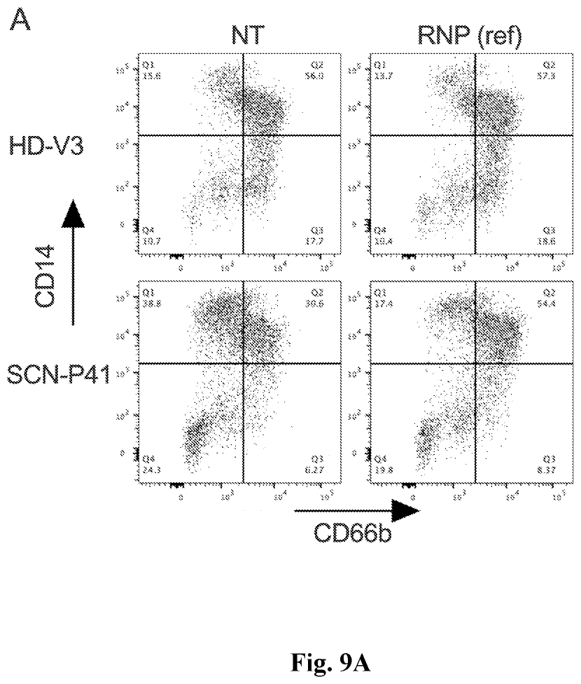

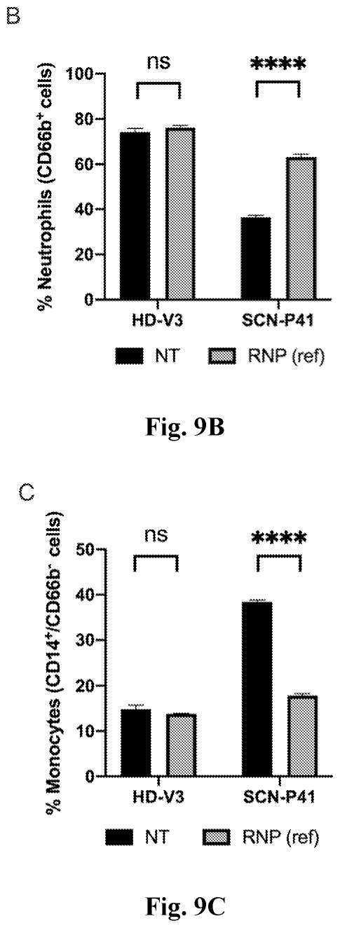

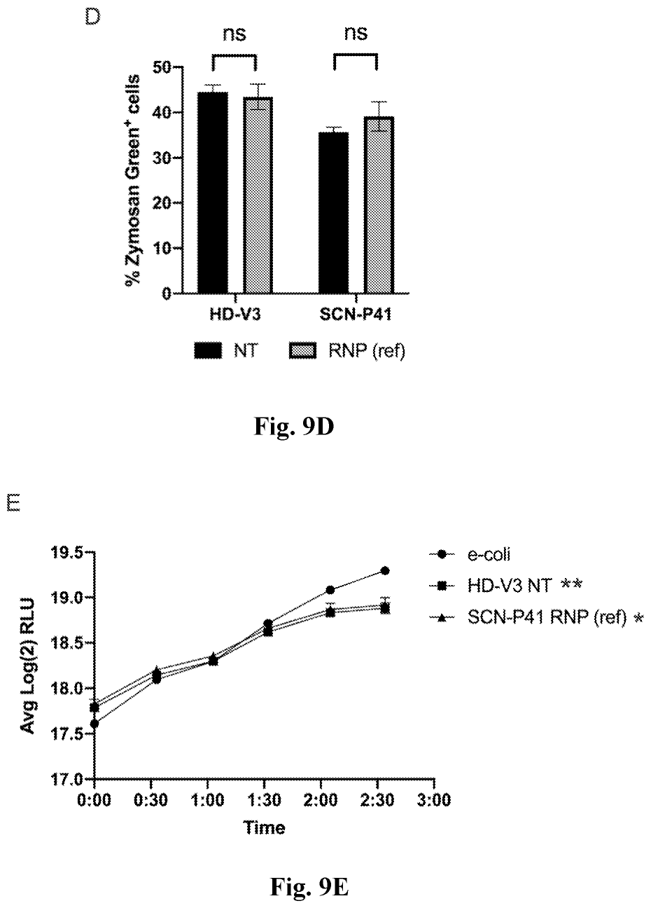

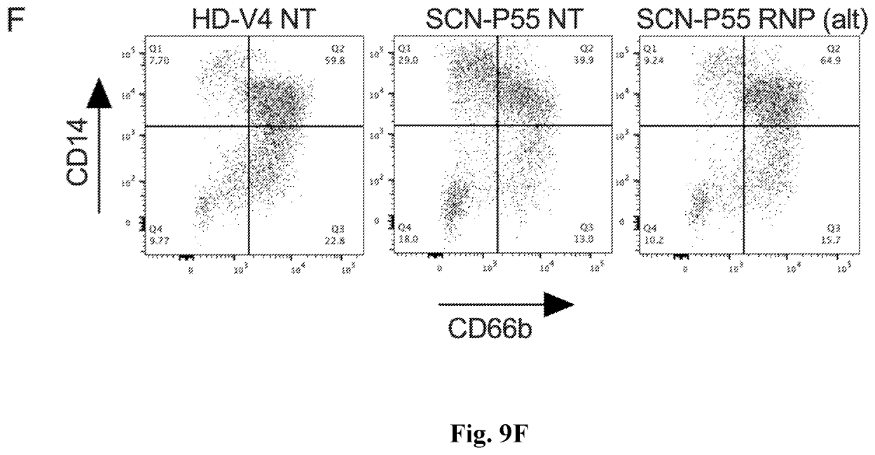

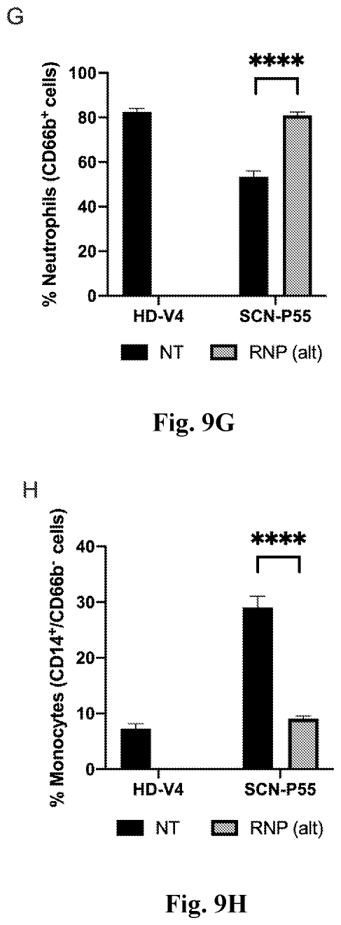

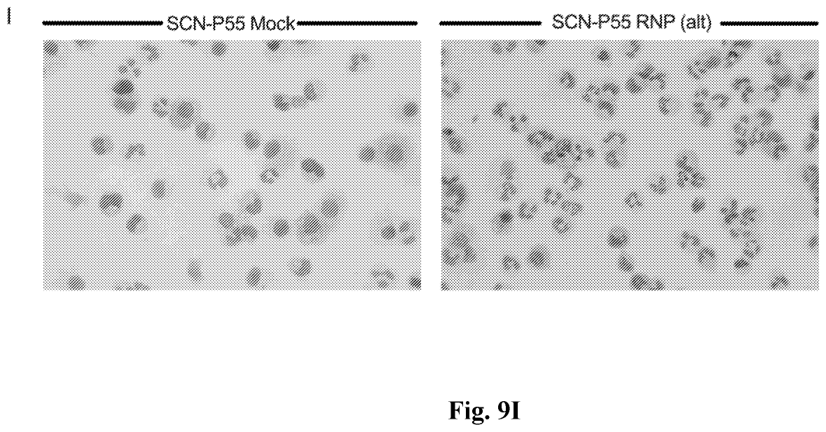

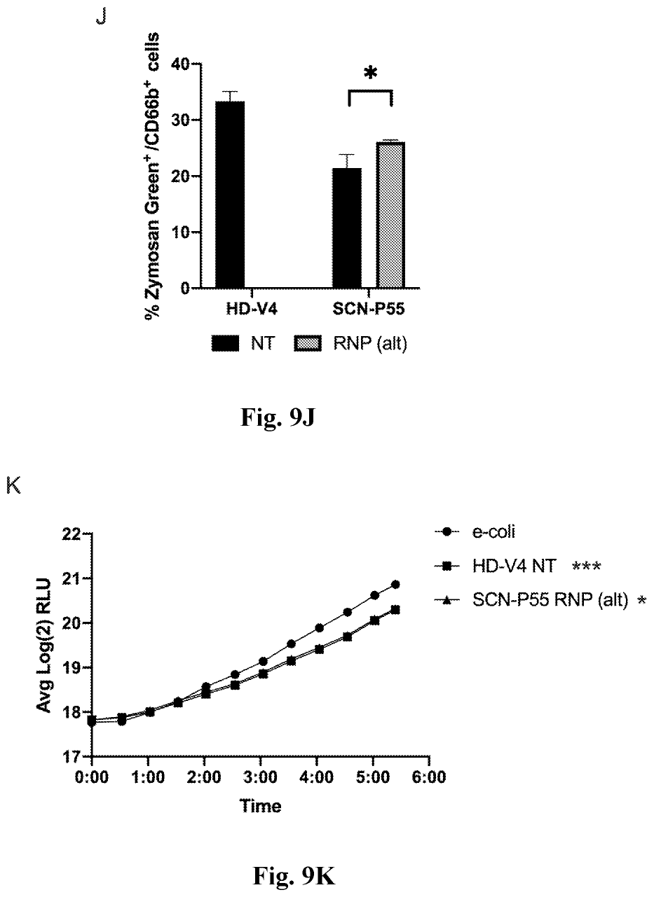

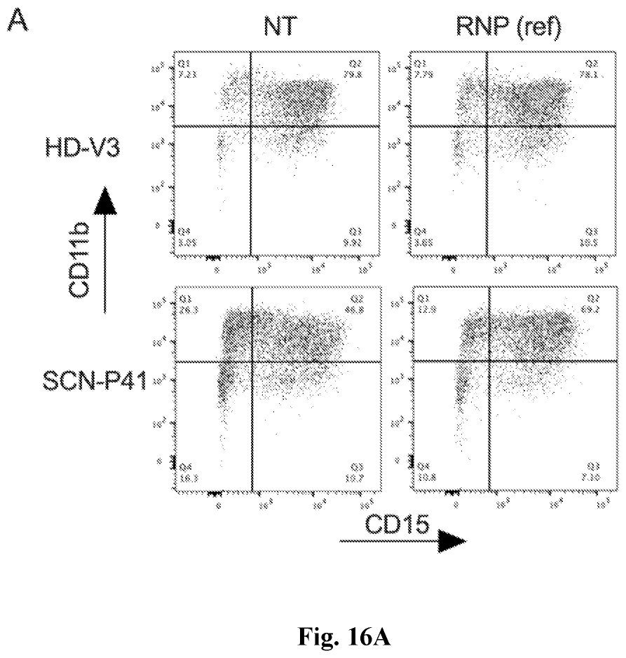

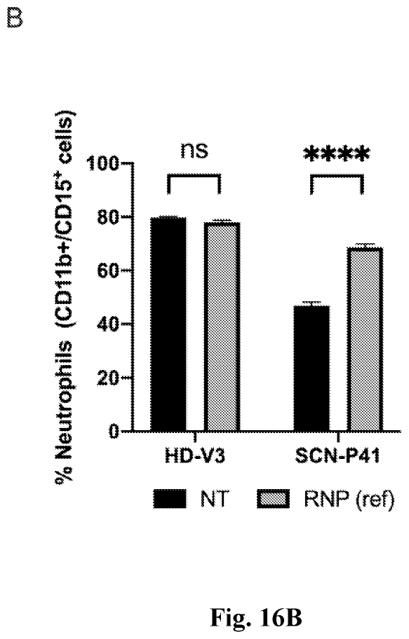

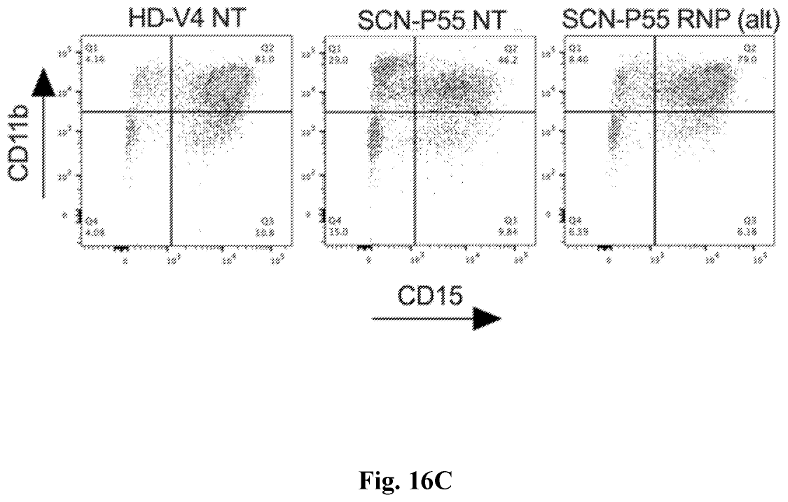

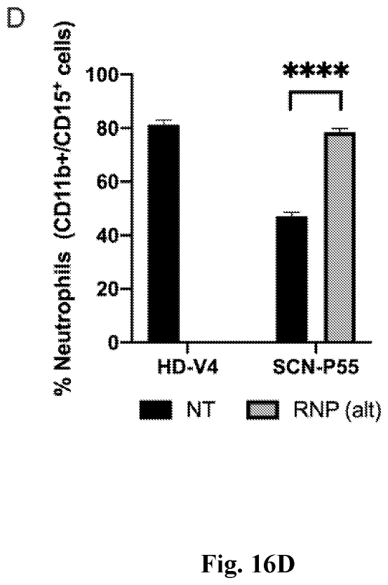

OMNI Variant 3795 facilitated editing boosts neutrophil differentiation and maturation in vitro. To demonstrate the functional outcome of our SNP-based single-allelic editing approach we evaluated neutrophil differentiation and maturation capacities of RNP(ref)-edited and non-treated healthy and patient-derived HSCs in vitro, at day 14 of differentiation. Flow cytometric analysis showed that about 74% of the HD-V3 HSCs, subjected to the differentiation protocol, differentiated into neutrophils (CD66b⁺; two right quarters (Q2+Q3) of the dot plot), compared to only 36% of the SCN-P41-derived HSCs. To the contrary, only about 15% of HD-V3 HSCs differentiated into monocytes (CD14⁺/CD66b⁻; upper left quarter (Q1) of the dot plot) compared to 38% of the SCN-P41-derived HSCs. These observations are consistent with the characteristic hematopoietic defect in SCN patients. (FIGS. 9A-9C; NT: HD-V3 versus SCN-P41). SCN-P41-derived HSCs treated with RNP(ref) depicted a 74% increase in neutrophils (CD66b⁺ cells) and a 2 fold reduction in the monocytic subset (CD14⁺/CD66b⁻) (FIGS. 9A-9C; SCN-P41: NT versus RNP(ref)). Similar increase in neutrophil count was observed in SCN-P41-derived edited HSCs by flow cytometric analysis of CD11b⁺/CD15⁺ cells (FIGS. 16A and 16B). RNP(ref)-mediated editing in HSCs of HD-V3 did not affect the monocytic and neutrophilic subsets, supporting the safety of this composition (FIGS. 9A-9C; HD-V3: NT versus RNP(ref)).

After demonstrating our allele-specific editing approach significantly improved the cellular abnormalities associated with SCN, we next assessed neutrophilic functions in RNP(ref)-treated and non-treated healthy (V3) and patient (P41) HSC-derived neutrophils. In vitro phagocytic capacity was tested by measurement of phagosomal uptake of zymosan green particles by neutrophils from the different groups. Flow cytometric analysis revealed equivalent levels of phagocytosis in RNP(ref)-treated and non-treated neutrophils from both HD-V3 and SCN-P41 (FIG. 9D). In addition, anti-bacterial killing capacity was examined by incubating neutrophils derived from non-treated healthy HSCs (HD-V3, NT) and RNP(ref)-treated patient HSCs (SCN-P41, RNP(ref)) with E-coli bacteria, expressing bacterial luciferase gene, and tracking real time changes in light emission, expressed as relative light units (RLUs). The two groups were compared with bacteria only (E-coli control without neutrophils). Healthy and RNP(ref)-treated patient derived neutrophils exhibited efficient bacterial killing as indicated by a 23-25% decrease in bacterial unit (RLU) in comparison to bacteria only control. Experiment was performed with 50,000 and 100,000 HSCs (FIG. 9E).

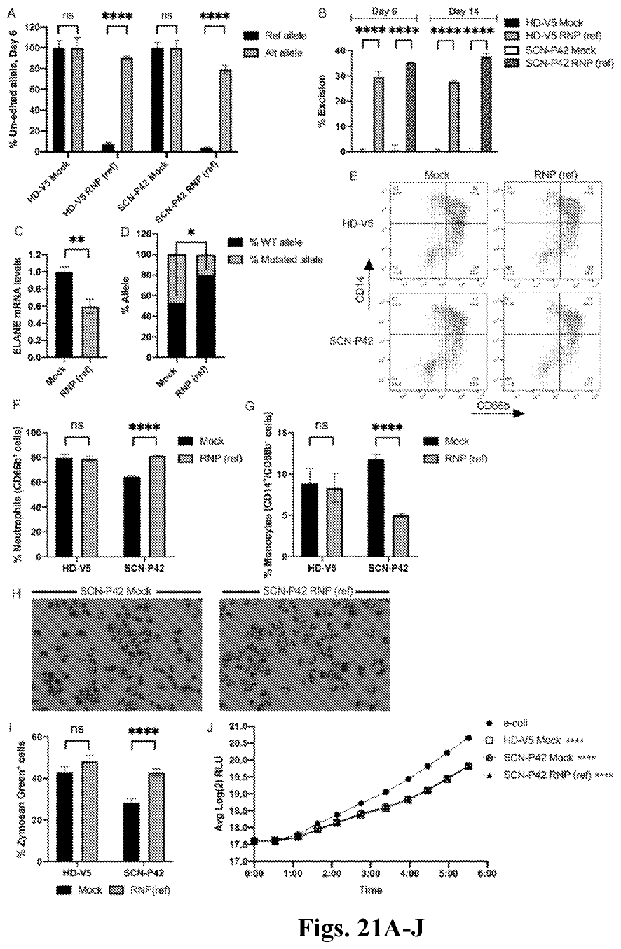

An experiment using the RNP(ref) composition was conducted on cells from another patient (SCN-P42) and depicted similar excision, differentiation and functional results, including histological staining demonstrating the restoration of neutrophil differentiation (FIGS. 21A-21J). Thus, specific excision of ELANE mutated allele by RNP(ref) composition ameliorated the aberrant phenotype of attenuated differentiation towards neutrophils and preserved neutrophil basic functions. A similar analysis was performed on the RNP(alt) composition. Flow cytometric analysis showed about 83% of HD-V4-derived HSCs differentiated into neutrophils (CD66b⁺; two right quarters (Q2+Q3) of the dot plot) and about 7% differentiated into monocytes (CD14⁺/CD66b⁻; upper left quarter (Q1) of the dot plot). SCN-P55-derived HSCs showed lower differentiation towards neutrophils (about 53%) and higher differentiation to monocytes (about 29%) (FIGS. 9F-9H; NT: HD-V4 versus SCN-P55), representing a typical SCN hematopoietic defect. SCN-P55-derived HSCs treated with RNP(alt) presented a 1.5 fold increase in neutrophils (CD66b⁺ cells) and a 3 fold reduction in the monocytic subset (CD14⁺/CD66b⁻) (FIGS. 9F-9H; SCN-P41: NT versus RNP(alt)). A similar increase in neutrophil subset was observed in SCN-P55-derived edited HSCs by flow cytometric analysis of CD11b⁺/CD15⁺ cells (FIGS. 16C and 16D). Diff-Quik staining of SCN-P55-derived HSCs treated with RNP(alt) or electroporated without a nuclease composition (SCN-P55 Mock) revealed higher numbers of cells with classical polymorphonuclear neutrophilic morphology in the RNP(alt)-treated group compared with Mock group (FIG. 9I). Flow cytometric analysis of zymosan green particles revealed slightly higher levels of phagocytosis in RNP(alt)-treated neutrophils compared to non-treated neutrophils from SCN-P55 (FIG. 9J). In addition, healthy non-treated (HD-V4, NT) and patient RNP(alt)-treated (SCN-P55, RNP(alt)) neutrophils exhibited efficient bacterial killing as indicated by a significant 30% decrease in bacterial unit (RLU) in comparison to bacteria only control (FIG. 9K).

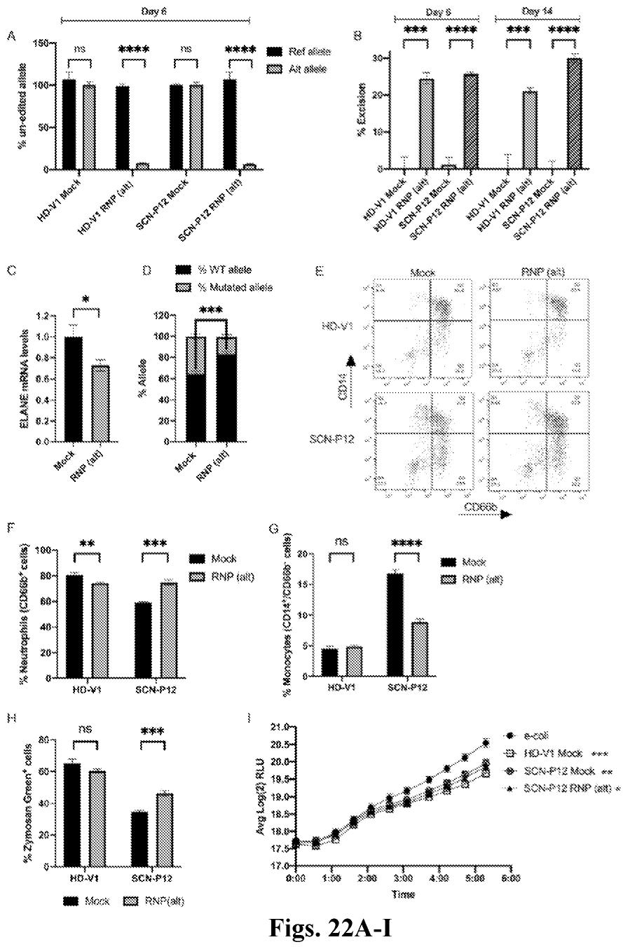

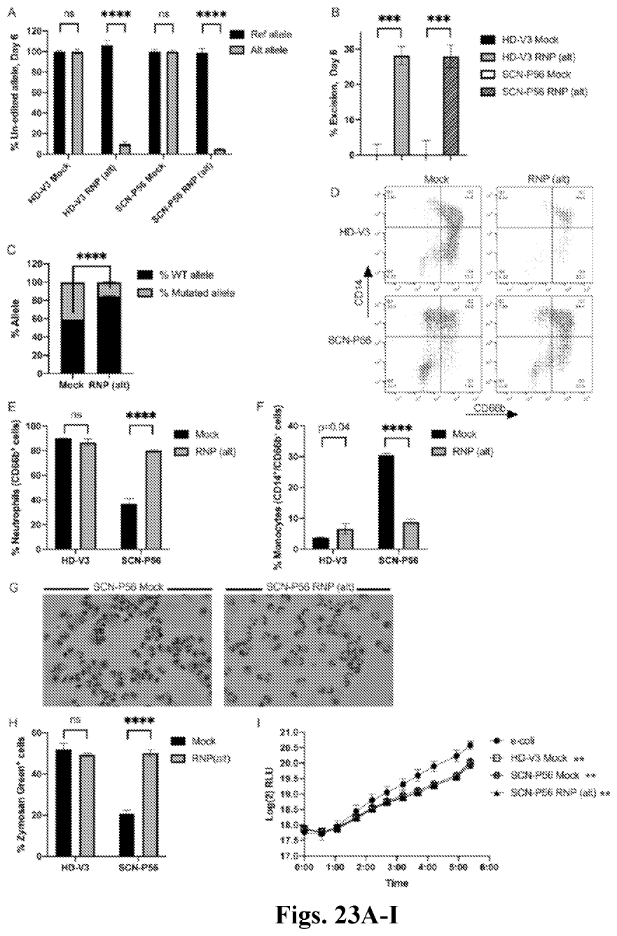

An experiment using the RNP(alt) composition was conducted on cells from another patient (SCN-P12) and depicted similar excision, differentiation and functional results (FIGS. 22A-22I). Moreover, another experiment performed on patient cells (SCN-P56) harboring a mutation on exon 2, located upstream to the mutations found in previous patients (exons 4 and 5), showed similar results (FIGS. 23A-23I). The results described herein indicate a single-allelic knock-out of ELANE mutated allele, using RNP(ref) and RNP(alt) novel nuclease compositions, is safe and effective both in boosting neutrophil differentiation and in maintaining essential neutrophilic core functions.

OMNI Variant 3795 showed high fidelity without traceable off targets. This is the first report demonstrating specific mono-allelic gene editing, which is not mediated by a gain of PAM sequence. This unique feature of our novel nuclease could be implemented in various indications that are dominant, dominant negative, and compound heterozygous, covering most genetic disorders that other technologies cannot address.

OMNI Variant 3795 composition showed excision efficiency of about 25%. Notably, given the high allele specificity of this nuclease composition, it is estimated that about 50% of the cell population had undergone excision at the mutant allele. With respect to the functional aspects, ELANE mono-allelic excision significantly enhanced neutrophil differentiation in-vitro. It also reduced the aberrant numbers of monocytes, consistent with the hematopoietic defect of SCN patients. Excised neutrophils showed normal phagocytic and bacterial killing capacities, indicating the editing was both effective and safe.

Thus, this Example presents a novel CRISPR/Cas9-based strategy of specific mono-allelic excision. Such a strategy was found to be efficient, functional, accurate and safe.

### Example 3: Materials & Methods

Human HSC isolation: HSCs were isolated from SCN patients' bone marrow and healthy donors' mobilized peripheral blood.

CRISPR/Cas9 OMNI Variant 3795 ELANE gene editing: Ribonucleic protein (RNP) system at a molar ratio of 1:2.5 (OMNI Variant 3795 nuclease:sgRNA) was used. Human CD34⁺ cells were electroporated using the CA-137 program (Lonza 4D, Nucleofector™.

Digital Droplet PCR (excision, allele specificity): Excision and allele specificity were measured using Digital Droplet PCR™ on genomic DNA. For excision reaction, amplification of two regions in ELANE gene, exon 1 and exon 5 was performed, using two different probes FAM(X1) and HEX(X1), respectively. The ratio between the probe signals was translated to excision efficiency. For allele specificity, a FAM probe (binding the alternative allele) and a HEX probe (binding the reference allele) were used (FAM+HEX). The ratio between the two probes was normalized to endogenous genes.

Assessment of mutated:wild-type allele ratio: cDNAs were mapped using next-generation sequencing (NGS) targeting exon 4 and 5 harboring S126L and R220Q mutations in patients 41 and 55, respectively. Relative ratio of mutated:wild-type alleles in treated cells was calculated and compared to that of non-treated cells.

Differentiation assay: Edited and non-treated HSCs were subjected to a differentiation protocol adopted from Nasri et al. On day 14, cells were analyzed by flow cytometry for monocytic (CD14⁺/CD66b⁻) and neutrophilic (CD66b⁺) subsets.

Bacterial killing assay: Day 13 differentiated HSCs were evaluated for their bacterial killing capacity as described in J. T. Atosuo. Relative light units (RLUs) were measured over 5 hrs. Last time point presented is when RLU levels reached plateau. Wells without differentiated HSCs (E. coli only) and with phagocytosis inhibitor, Cytochalasin D, (data not shown) served as controls.

Phagocytosis assay: Phagocytosis capacity was evaluated using the EZCell™ Phagocytosis Assay Kit (Green Zymosan), (BioVision, Cat no. K397). Cells were analyzed by flow cytometry for internalization of opsonized fluorescent Zymosan Green particles.

Further details on FACS antibodies, probes, sequences, assays, protocols, and additional methods are provided in Supplemental Methods.

### Example 3: Supplemental Methods

Human SCN Patient HSC isolation: Three to 6 mls of freshly collected bone marrow was shipped overnight at ambient temperature. Hematopoietic stem and progenitor cells, HSPC's, were initially enriched using RosetteSep Human Bone Marrow Progenitor Cell Pre-Enrichment Cocktail, (Cat. No. 15027) and Lymphoprep (Cat. no. 07801) according to manufacturer's protocol. The HSC enriched cell population was expanded by culturing for 4 days in CD34⁺ expansion media (StemSpan SFEMII media (Cat. no. 09655) supplemented with 1% Penn Strep (Cat. no 03-031-1B, Biological Industries), 1× StemSpan CD34 Expansion Supplement (10×) (Cat. no. 02691), and 1.0 μM UM729 (Cat. no. 72332), at 37° C. 5% CO₂. After expansion, CD34⁺ cells were further enriched using EasySep Human CD34 Positive Selection Kit II (Cat. no. 17856) according to manufacturer's protocol. Enriched CD34⁺ cells were cryopreserved at 1×10⁶ cells/ml in Cryostor CS10 (cat. no. 07931). Cells were stored in liquid nitrogen, vapor phase. All catalog numbers refer to materials from StemCell Technologies unless indicated otherwise.

Human healthy donor HSC isolation: Cryopreserved healthy human CD34⁺ progenitor cells from mobilized peripheral blood were obtained from Lonza (Cat no. 4Y-101C). Cells were suspended in CD34⁺ expansion media at 50,000 cells/ml and expanded for 4 days at 37° C., 5% CO₂ prior to electroporation.

CRISPR/Cas9 OMNI Variant 3795 ELANE gene editing: Editing of HSCs was carried out using a ribonucleic protein (RNP) system at a molar ratio of 1:2.5 (nuclease:sgRNA), including 17 μg nuclease and 262 pmol of each guide. Nuclease and sgRNA complex were incubate at 25° C. for 10 minutes. Human CD34⁺ cells were washed once with PBS. 2×10⁵ CD34⁺ cells were suspended into 20 μl of P3 electroporation buffer (Lonza P3 kit S) and were added to RNPs mix. After electroporation, using the CA-137 program (Lonza 4D, Nucleofector™), the cells were transferred to pre-warmed CD34⁺ expansion media at a concentration of 1.25.0×10⁵ cells/ml. Guides were manufactured by Agilent. Guide sequences are summarized in the table below:

| sgRNA name | Guide sequence (scaffold + spacer) |
|---|---|
| sgRef | GUGUCAAGCCCCAGAGGCCACAgUUUGAGAGUUA |
| | UGAAAAUGACGAGUUCAAAUAAAAAUUUAUUCAA |
| | ACCGCCUAUUUAUAGGCCGCAGAUGUUCUGCUUU |
| | (SEQ ID NO: 35) |
| sgAlt | GUGUCAAGCCCCAGAGGACACAgUUUGAGAGUUA |
| | UGAAAAUGACGAGUUCAAAUAAAAAUUUAUUCAA |
| | ACCGCCUAUUUAUAGGCCGCAGAUGUUCUGCUUU |
| | (SEQ ID NO: 36) |
| sgConstant | GCAGUCCGGGCUGGGAGCGGGUgUUUGAGAGUUA |
| | UGAAAAUGACGAGUUCAAAUAAAAAUUUAUUCAA |
| | ACCGCCUAUUUAUAGGCCGCAGAUGUUCUGCUUU |
| | (SEQ ID NO: 37) |

Digital Droplet PCR for percentage excision and allele specificity: Percentage excision and allele specificity were measured using Digital Droplet PCR™ (ddPCR™, Bio-Rad, Hercules, CA, USA) on genomic DNA that was extracted using QIAamp DNA Micro Kit, Qiagen (Cat no. 56304). According to manufacturer's protocol.

ddPCR reaction contained 1× ddPCR Supermix for probe without dUTP (#1863024), 25-100 ng of digested DNA using HindIII (diluted in X1 Cutsmart Buffer to 4 U/μL) and suitable primers/probes. For excision reaction, amplification of two regions in ELANE gene, exon 1 and exon 5 was performed, using two different probes labeled with FAM (X1) and HEX (X1), respectively. The ratio between the HEX and the FAM signals was translated to excision efficiency.

For allele specificity, two competitive probes: a FAM probe, which binds the alternative allele, and a HEX probe, which binds the reference allele were used (FAM+HEX). The ratio between the concentrations of the two in heterozygote non-treated cell is 1, which was normalized to the endogenous genes RPP30 and STAT1 for each gDNA sample. Reaction total volume was 22 μL. The binding of each probe to DNA extracted from healthy donor cells that were homozygous to either the reference or alternative forms of the SNP was measured by ddPCR, confirming the probes do not cross react, (FIG. 19A). Moreover, healthy donor cells homozygous to the reference form of the SNP depicted efficient excision when treated with RNP(ref) composition, compared to treatment with RNP(alt) composition that resulted in excision levels comparable to non-treated cells. This further demonstrates the specific targeting of the sgRNAs (FIG. 19B).

Genomic DNA in the ddPCR mixture was partitioned into individual droplets using QX100 Droplet Generator, transferred to a 96-deep well PCR plate and amplified in a Bio-Rad PCR thermocycler. Bio-Rad Droplet Reader and QuantaSoft Software were used to read and analyzed the experiment following manufacturer's guidelines (Bio-Rad).

The primers and probes were manufactured by Bio-Rad and are detailed in the table below:

| Probe name | Catalog number |
|---|---|
| ELANE ddPCR assay | dHsaCNS328407057 |
| Exon1 (FAM) | |
| ELANE ddPCR assay | qHsaCEP0055470 |
| Exon5 (HEX) | |
| rs1683564 Editing | dHsaMDS873573221 |
| (FAM + HEX) | |
| ddPCR assay | |
| RPP30 | dHsaMDS117591774 |
| STAT1 | dHsaCNS850507320 |

Assessment of mutated:wild-type allele ratio: cDNAs from HSCs treated with either RNP(ref) or RNP(alt) were mapped using next-generation sequencing (NGS) targeting exon 4 and 5 harboring S126L and R220Q mutations in patients 41 and 55, respectively. The raw FASTQ files were analyzed, and BAM files (text-based format for storing biological sequences) were generated using FASTQ to BAM script. The relative ratio of the mutated allele to the wild-type allele was calculated and compared to the non-treated cells in both patients. Primers are detailed in the table below:

| Mutation | Sequence |
|---|---|
| S126L | TCGTCGGCAGCGTCAGATGTGTATAAGA |
| (P41) | GACAGNNNTACGACCCCGTAAACTTGCT |
| | GTCTCGTGGGCTCGGAGATGTGTATAAG |
| | AGACAGNNNCGGAGCGTTGGATGATAGA |
| | G |
| R220Q | TCGTCGGCAGCGTCAGATGTGTATAAGA |
| (P55) | GACAGNNNTCGCAGTCCAGCTTCCCCAC |
| | GTCTCGTGGGCTCGGAGATGTGTATAAG |
| | AGACAGNNNACAGCCAAGGAGCATCAAA |
| | C |

Differentiation assay: Edited and non-treated HSCs were allowed to recover for 3 days in CW34⁺ expansion media and were subjected to a differentiation protocol adopted from Nasri et al.²³ In brief, HSCs were cultured for 7 days in RPMI (Cat. no 11875093, Gibco™) supplemented with 1% Glutamax (Cat. no 35050061, Gibco™), 10% FBS (Cat. no 04-001-1A, Biological Industries), 5 ng/ml IL-3 (Cat. no 200-03), SCF (Cat. no 300-07), GM-CSF (Cat. no 300-03) & 10 ng/ml G-CSF (Cat. no 300-23), all from PeproTech, for proliferation and myeloid progenitor differentiation followed by a 7-day culture in RPMI, 1% Glutamax, 10% FBS, 1% Penn Strep (Cat. no 03-031-1B, Biological Industries), 10 ng/mil G-CSF for neutrophil differentiation and maturation. On day 14 cells were analyzed by flow cytometry for monocytic (CD14⁺/CD66b⁻) and neutrophilic (CD66b⁺) subsets. CD66b anti-human, Pacific Blue (Cat No. 305112, Biolegend) and CD14, anti-human, APC (Cat No. 130-110-520, Miltenyi Biotec) were used.

Cytospin staining: 8×10⁴ HSCs at day 15 of differentiation were spun onto Cytoslide microscope slides (ThermoFisher) using Cytospin 4 low speed cytocentrifuge (Thermo Scientific) and stained with Diff-Quick staining system (MilliporeSigma) according to manufacturer's recommendations. Microphotographs were taken on LEITZ LABORLUX S polarizing light microscope at 400× magnification using Nikon DSLR digital camera.

Bacterial killing assay: Day 13 differentiated HSCs (subjected to a differentiation protocol adopted from Nasri et al.) from healthy donors and SCN patients (edited with either RNP(ref), RNP(alt) or non-treated), were evaluated for their bacterial killing capacity as described in J. T. Atosuo. Briefly, 100,000 differentiated HSCs were incubated in the presence of 200,000 Luciferase expressing bacterial cells, pAKLUX2, per well (Addgene, Cat No. 14080). Cells were cultured in 200 ul HBSS++, 10% FBS at 37° C. At 30-minute intervals luminescence was measured by transferring the plate to a Luminometer (Berthold CentroXS3 LB960) and measuring luminescence for 0.5 see per well). A real time change in light emission, relative light units (RLUs), was measured over 5 hrs. Last time point presented is when RLU levels reached plateau. Wells without differentiated HSCs (E. coli only) and with 10 ng/ml phagocytosis inhibitor (data not shown), Cytochalasin D (Santa Cruz Biotec, Cat No. sc-20144) served as controls.

Phagocytosis assay: Phagocytosis capacity was evaluated using the EZCell™ Phagocytosis Assay Kit (Green Zymosan), (BioVision, Cat no. K397 according to manufacturer's protocol). Day 14 differentiated HSCs (subjected to a differentiation protocol adopted from Nasri et al.) from healthy donors and SCN patients (edited with either RNP(ref), RNP(alt) or non-treated), were resuspended in HBSS++/10% FBS (0.5×10⁶ cells/ml) and incubated for 1.5 hours at 37° C. in the presence of 5 ml opsonized Alexa Fluor 488-conjugated zymosan particles per 200 ml suspended cells. As a negative control, cells were incubated with 10 ng/nil of the phagocytosis inhibitor, Cytochalasin D (Santa Cruz Biotec, Cat No. sc-201442) 1 hour prior and during incubation with Zymosan Green reagent. Cells were then washed and incubated in a quencher solution, based on kit's instructions, to remove fluorescence from particles that were not internalized. Cells were then analyzed by flow cytometry for internalization of opsonized fluorescent Zymosan Green particles.

Inversions: Inversion events were detected and quantified by a Droplet Digital PCR (ddPCR) mutation assay. First, a perfect inversion was mimicked using a SnapGene software and verified by NGS. Then, total inversion events were quantified by ddPCR using EvaGreen dye, a fluorescent DNA-binding dye that binds dsDNA (BIO-RAD, catalog number 186-4034, according to manufacturer's protocol). Specific primers were designed to amplify inverted variations of the excised fragment (See illustration in FIG. 11). Fluorescent signals were normalized to amplification of ELANE exon 1 region that was not affected by excision (performed by two different sets of primers. Averaged normalized data is presented). Primers are detailed in the table below:

| Primer name | Sequence |
|---|---|
| Exon 4 primer | ACGTCTGCACTCTCGTGAGG |
| Exon 5 primer | TTCAGGCTCCACCCAGTTTGTC |
| (inverted) | |
| Exon 1 primers | Mix3: |
| | GCACAGGGCTATAAGAGGAGC |
| | GCGGGAGGTTGGACTCAAAA |
| | Mix6: |
| | GGGAGAGGAAGTGGAGGGC |
| | GAGGGTCATGGTGGGGCT |

Excision levels in Long Term HSC population: HSCs of two healthy donors (MLP1 (3055934); heterozygous to the alternative form of the SNP and MLP2 (3055940), homozygous to the alternative form of the SNP), isolated from leukopaks purchased from AllCells, were edited a day after thaw according to ‘CRISPR/Cas9 OMNI Variant 3795 ELANE gene editing’ section above, with minor changes. Cell number was 2M cells/electroporation. Upscale of guides and nuclease was performed accordingly. A molar ratio of 1:2.5 (nuclease:sgRNA) was used including 85 μg nuclease and 1310 pmol of each guide. Nucleofection was performed in P3 nucleofection solution (Lonza) and Lonza 4D-Nucleofector™ X Kit L (program CA-137). Three days after editing, cells were sorted in a FACS ARIA™ II SORP Flow Cytometer Cell (BD), using CD90-APC-Vio770, human (130-114-863 Milteny). Sorted cells were incubated for 7 days in a proliferation medium according to Nasri et al. Excision levels of CD90⁺, CD90⁻ and total population were evaluated using ddPCR as described in ddPCR section.

Mutation-SNP linkage: First, ELANE mutation and possible SNPs were identified in cells from SCN patients by targeted short read NGS. Then, a part of the gene encompassing both the mutation and the SNP was amplified by a PCR reaction with linkage primers and cloned into bacteria. Each clone bore an amplicon from one allele. A plasmid of multiple clones was Sanger sequenced (with T7 and SP6 primers) for the mutation and the SNP regions. If the mutation and the SNP were in cis, they were found at the same clone, if in trans, the mutation and SNP were found in different clones. Primers are detailed in the table below:

| Primer | |
|---|---|
| name | Sequence |
| rs10414837 | FP: |
| | TCGTCGGCAGCGTCAGATGTGTATAAGAGA |
| | CAGNNNGGTGGGTCCTCAGTGACTCT |
| | RP: |
| | GTCTCGTGGGCTCGGAGATGTGTATAAGAG |
| | ACAGNNNGGGAATTCCAGCCTGACCAA |
| rs3761005 | FP: |
| | TCGTCGGCAGCGTCAGATGTGTATAAGAGA |
| | CAGNNNGAGTGAGGACCAAGCCTGAG |
| | RP: |
| | GTCTCGTGGGCTCGGAGATGTGTATAAGAG |
| | ACAGNNNAGGGCCATTGTCTCCCTAAC |
| rs1683564 | FP: |
| | TCGTCGGCAGCGTCAGATGTGTATAA |
| | GAGACAGNNNTCCTGCTACCCTCCCTTCTT |
| | RP: |
| | GTCTCGTGGGCTCGGAGATGTGTATA |
| | AGAGACAGNNNTTTTAGGAGGGGCCCACTG |
| | A |
| Linkage | FP: |
| | GAGGGTCATCATCACTGCCC |
| | RP: |
| | GCCAGACTCACACCAGAGTCGACAAG |
| | T |
| T7 | TAATACGACTCACTATAGGG |
| SP6 | ATTTAGGTGACACTATAG |
| R220Q | FP: |
| | TCGTCGGCAGCGTCAGATGTGTATAAGAGA |
| | CAGNNNTCGCAGTCCAGCTTCCCCAC |
| mutation | RP: |
| | GTCTCGTGGGCTCGGAGATGTGTATAAGAG |
| | ACAGNNNACAGCCAAGGAGCATCAAAC |
| S126L | FP: |
| | TCGTCGGCAGCGTCAGATGTGTATAAGAGA |
| | CAGNNNGAGGGTCATCATCACTGCCC |
| mutation | RP: |
| | GTCTCGTGGGCTCGGAGATGTGTATAAGAG |
| | ACAGNNNAGTCCGGGCTGGGAGCGGGT |

Heterozygosity frequency of SNPs in the healthy and patient populations: Variant call files encompassing the ELANE gene region (±3 kb of ELANE gene) were downloaded from the 1000 Genomes Project Consortium (phase 3) using the Data Slicer tool and analyzed in the R statistical computing environment. 3501 genotypes were available from 3501 individuals. Familial relationship was omitted from the analysis, which resulted in 2407 genotypes from unrelated individuals. The allele frequency for all common polymorphism (>1% MAF) was calculated. Three SNPs were chosen (rs3761005, rs1683564, and rs10414837 polymorphisms) to optimize the population coverage for allele-specific ELANE knock-out. The percentage of the population being heterozygous for at least one of the three chosen SNPs was calculated.

53 patients' samples were sequenced for the pathogenic mutation and the three chosen SNPs. A total of 46 bone marrow samples and 7 iPSC lines were used. 44 of the samples were from SCN patients and nine of them were from patients with Cyclic Neutropenia. Heterozygosity frequency of each of the three chosen SNPs and the percentage of the population being heterozygous for at least one of them was calculated.

Statistical methods: The two-sample T-test for independent samples or the ANOVA model, as appropriate, was applied for testing the statistical significance of the difference in continuous variables between treatment groups. The two-ways Analysis of Variance with repeated measurements was used to analyze killing assays. Chi-square test or Fisher's Exact test, as appropriate, was applied to test the statistical significance of the difference in heterozygosity between healthy and patient populations. All tests were two-tailed, and a p-value of 5% or less was considered statistically significant. The data was analyzed using Prism software (GraphPad version 9.0.2).

SUPPLEMENTAL TABLE 1

| Patient | | | ELANE | Location of | | | |
|---|---|---|---|---|---|---|---|
| # | Diagnosis | Source | mutation | mutation | rs10414837 | rs3761005 | rs1683564 |
| 1 | SCN | Bone Marrow | S126L | 4 | CC | TA | CC |
| 2 | SCN | Bone Marrow | G210V | 5 | CT | TT | CC |
| 3 | SCN | Bone Marrow | C555 | 2 | CC | TA | CC |
| 4 | SCN | Bone Marrow | G221ter | 5 | CC | TA | AA |
| 5 | SCN | Bone Marrow | D230fs | 5 | CC | AA | CC |
| 6 | SCN | Bone Marrow | A57T | 2 | CT | TA | AA |
| 7 | SCN | Bone Marrow | R103P | 3 | CC | TT | AA |
| 8 | SCN | Bone Marrow | T128del | 4 | CT | AA | CA |
| 9 | SCN | Bone Marrow | V83D | 3 | CT | TT | AA |
| 10 | SCN | Bone Marrow | L84P | 3 | CT | TA | CA |
| 11 | SCN | Bone Marrow | L8RP | 3 | CT | TT | CA |
| 12 | SCN | Bone Marrow | M154R | 4 | CC | TT | CA |
| 13 | SCN | Bone Marrow | G221ter | 5 | CT | TT | AA |
| 14 | SCN | Bone Marrow | G203R | 5 | CT | TA | CA |
| 15 | SCN | Bone Marrow | G214R | 5 | CC | AA | CA |
| 16 | SCN | Bone Marrow | P139L | 4 | CC | TA | CC |
| 17 | SCN | Bone Marrow | Y228tr | 5 | CT | TT | AA |
| 18 | SCN | Bone Marrow | P139L | 4 | CC | TA | CA |
| 19 | SCN | Bone Marrow | W156R | 4 | CT | TA | CC |
| 20 | SCN | Bone Marrow | P139L | 4 | CC | AA | CA |
| 21 | CyN | iPSC | P139L | 4 | CC | TT | CC |
| 22 | SCN | iPSC | I120N | 3 | CT | TT | AA |
| 23 | SCN | iPSC | G214R | 5 | CC | TA | CC |
| 24 | CyN | iPSC | S46F | 2 | CC | TA | CA |
| 25 | SCN | Bone Marrow | IVS4 + 5G > A | INT4 | CC | TT | CA |
| 26 | SCN | Bone Marrow | S126L | 4 | CC | TA | CA |
| 27 | SCN | Bone Marrow | R103L | 3 | CC | TA | CA |
| 28 | SCN | Bone Marrow | M66R | 2 | TT | TT | CC |
| 29 | SCN | Bone Marrow | C20BX | 5 | CT | TA | CC |
| 30 | SCN | Bone Marrow | M1R | 1 | CC | TT | CA |
| 31 | SCN | Bone Marrow | P234fs | 5 | CC | AA | CC |
| 32 | SCN | Bone Marrow | 160T | 2 | CC | TT | CA |
| 33 | CyN | iPSC | IVS4 + 1 sd | INT4 | CT | TT | CC |
| 34 | SCN | iPSC | P139L | 4 | CT | TT | AA |
| 35 | SCN | iPSC | R191S | 4 | TT | TT | CC |
| 41 | SCN | Bone Marrow | S126L | 4 | CC | AA | CA |
| 42 | SCN | Bone Marrow | S126L | 4 | CC | AA | CA |
| 43 | CyN | Bone Marrow | IVS4 + 5G > A | INT4 | CC | TT | CC |
| 44 | CyN | Bone Marrow | IVS4 + 1 G > A | INT4 | CT | TA | CA |
| 45 | CyN | Bone Marrow | R220Q | 5 | CT | TT | CC |
| 46 | CyN | Bone Marrow | IVS4 + 3A > T | INT4 | CC | TA | CC |
| 47 | CyN | Bone Marrow | W241ter | 5 | CT | AA | CC |
| 48 | SCN | Bone Marrow | S126L | 4 | CT | TA | CC |
| 49 | SCN | Bone Marrow | S126L | 4 | CT | TA | CC |
| 50 | SCN | Bone Marrow | P139L | 4 | CT | TA | CC |
| 51 | SCN | Bone Marrow | S46F | 2 | CC | TA | CC |
| 52 | SCN | Bone Marrow | IVS3 − 8 | INT3 | CT | TA | CC |
| 53 | SCN | Bone Marrow | P139L | 4 | CC | TA | CA |
| 54 | SCN | Bone Marrow | R220Q | 5 | CC | TA | CA |
| 55 | SCN | Bone Marrow | R220Q | 5 | CT | TA | CA |
| 56 | SCN | Bone Marrow | A57V | 2 | CT | AA | CA |
| 57 | SCN | Bone Marrow | R191S | 4 | TT | TT | CC |
| 58 | CyN | Bone Marrow | S126L | 4 | CC | AA | CA |

Supplemental Table 1. Coverage of the three SNPs by the patient population. Sequencing results of the three SNPs in ELANE gene in samples obtained from SCN and Cyclic Neutropenia (CyN) patients. The pathogenic mutations in ELANE gene were also verified by sequencing. Green cells depict heterozygous SNPs.

### Example 4: Contribution of Each of the Variant 3795 Mutations to the Specificity and Fidelity of the Nuclease

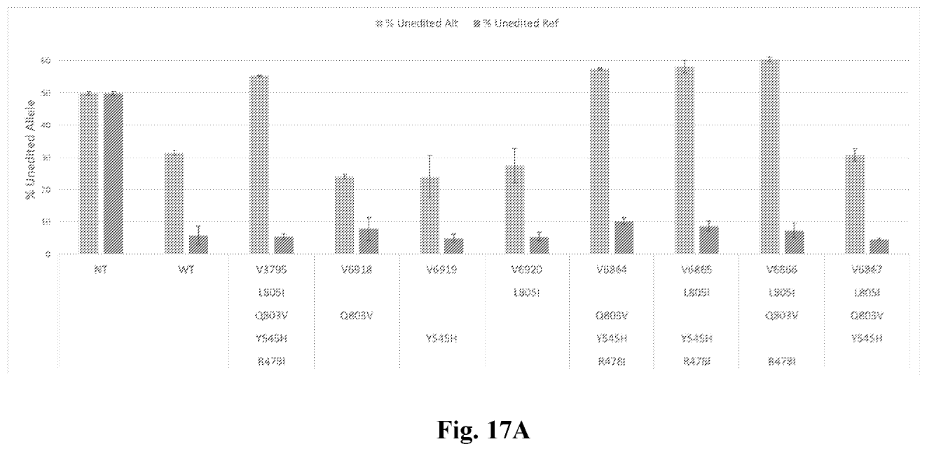

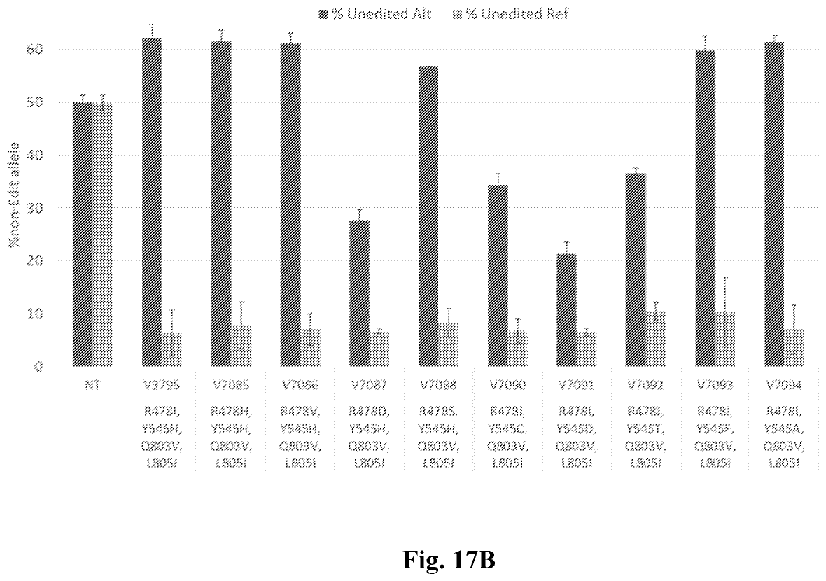

Variant 3795 is a specificity variant of CRISPR-based nuclease with extremely low off-target activity (<0.05%) and an ability to discriminate between two alleles of the same gene which have a single mismatch between them. The variant includes four mutations: R478I, Y545H, Q803V and L805I. To investigate the effect and contribution to the activity and specificity of each mutation found in OMNI-50 V3795, three out of the four single mutant variants were expressed and purified, as well as the four triple mutant variants, in which one of the four mutations is missing in each variant (Table A). RNP complexes were prepared using WT OMNI-50 nuclease, the V3795 variant, and the seven variants by assembly with sgRNA g62-Ref. These RNP complexes were electroporated into LCL cells that are heterozygote for the target of the guide sequence (Table B). The editing levels of the targeted allele (REF) and non-targeted allele (ALT) were measured by NGS. As can be seen in FIGS. 17A-17B, the discrimination towards the targeted allele (REF) of V3795 is contributed mostly by the R478I mutation (Table E). Only variants containing the R478I mutation (e.g. V6864, V6865, V6866, V3795) showed clear preference toward the targeted allele, while removing this mutation abolished the discrimination (e.g. V6867).

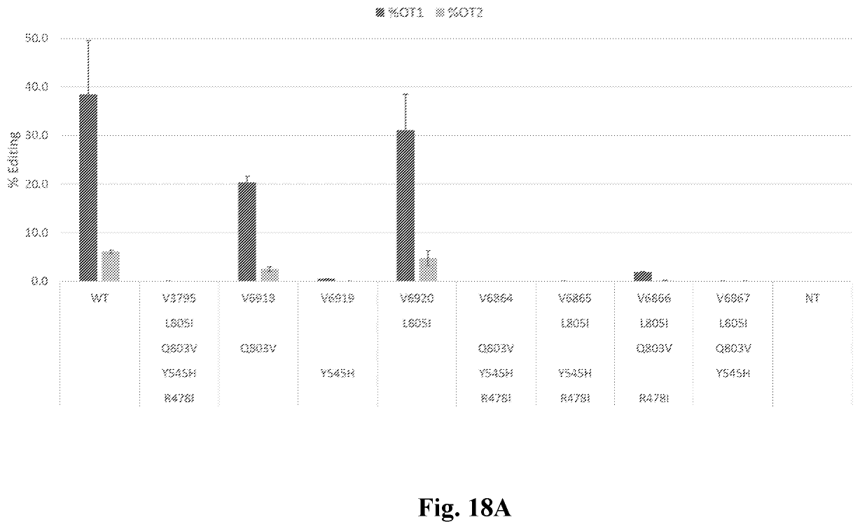

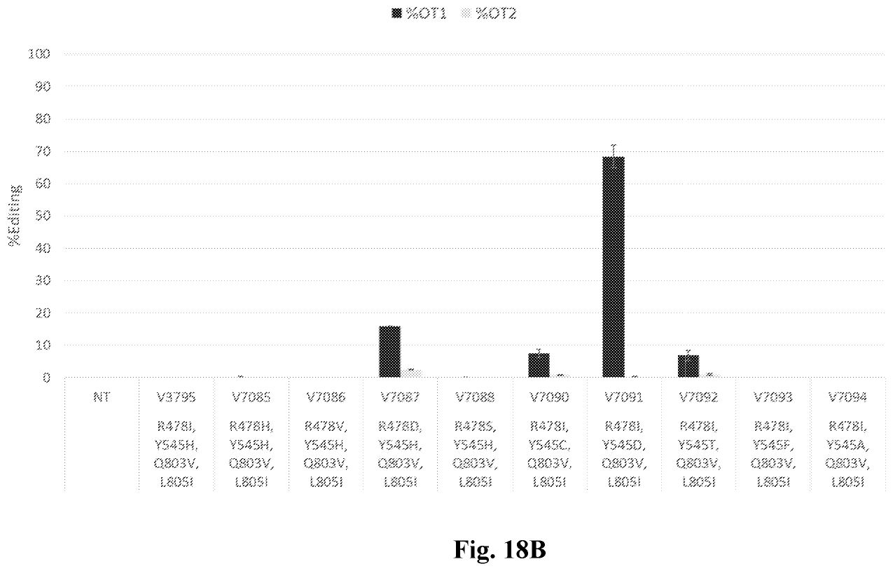

The specificity effect was validated by measuring the editing levels at two known off-target sites of g62 by NGS (g62OT1 and g62OT2, Table C). OMNI-50 V3795 has extremely low levels of editing at these sites in comparison to WT OMNI-50 nuclease (FIGS. 18A-18B, Table F). In all the three single mutants lower than WT editing levels of the off-target sites were observed, indicating a contribution of the three mutations to the reduction in off-targets. However, the Y545H mutation had the most significant reduction in the off-target editing (OT1 0.5% and OT2 0.04%, Table F). All of the triple variants also exhibited extremely low off-target activity, similar to the V3795 variant nuclease. Remarkably, when the Y545H mutation was excluded from the set of mutations (e.g. V6866) the off-target editing slightly increased (OT1 1.90% OT2 0.2%, FIGS. 18A-18B, Table F). Altogether, these results led to the conclusion that the V3795 variant nuclease's high level of specificity is attributed to the R478I and Y545H mutations.

Once it was established that R478I and Y545H are responsible for the observed specificity of the V3795 variant nuclease, the effect of other substitutions was tested. For each of these positions, RNPs were generated with the g62-Ref sgRNA and several variant nucleases containing substitutions from different biochemical groups in the background of the other three mutations (Table A). The effect on discrimination and off-targets was measured as above (FIGS. 17A-17B and 18A-18B).

Regarding position 478: Charged amino acids, such as aspartic Acid (D) in variant V7087, abolishes the discrimination and increases the off-target editing (FIG. 18B). A similar effect on discrimination is seen for arginine (R, the reference amino acid) in the triple variant V6867 (FIGS. 17A-17B). However, large hydrophobic amino acids such as valine (V) in V7086 as well as isoleucine (I) in V3795 retain high discrimination and reduced off-targets. An aromatic amino acid such as histidine (H) in V7085 and a polar uncharged amino acid such as serine (S) in V7088 also supports a similar phenotype as OMNI-50 V3795.

Regarding position 545: Substitution to amino acids such as phenylalanine (F) in V7093, as well as histidine (H) in V3795, retain high discrimination and low off-targets similar to the variant V3795 (FIGS. 17A-17B and 18A-18B). Alanine (A) is a small hydrophobic residue and is used as a substitute in in V7094, which also showed a similar phenotype as V3795 (FIGS. 17B and 18B).

### Example 4: Materials & Methods

### HTP Protein Expression and Purification

Wild-type OMNI-50 nuclease and its variants were cultured in auto-induction TB media at 37° C., 350 rpm agitation for 3.5 hours, and then shifted to 18° C. for 17-20 hours. Cells were harvested by centrifugation at 4000×g and stored at −80° C. OMNI-50 variants and WT OMNI-50 cell pellets were thawed and incubated in lysis buffer for 30 minutes. Crude lysate was clarified by centrifugation at 4000×g for 1 hour, 4° C. Cleared protein lysates were incubated with Sepharose 6 Ni-NTA resin (Cytiva). Protein-bound Ni-NTA resin was loaded on a 96 Filter well plate and washed with buffer (HEPES 20 mM, NaCl 0.6 M, Imidazole 60 mM) to remove contaminants. Protein was eluted from the resin with high concentrations of imidazole buffer (HEPES 20 mM, NaCl 0.6 M, Imidazole 0.4 M). Eluted proteins were desalted using 96 filter plates containing 1800 μl of G-25 Sephadex resin equilibrated with storage buffer. Desalted proteins were then concentrated (Amicon-ultra 0.5 ml 50 kDa, Millipore) and sterile filtered (Ultrafree-MC 0.22 m PVDF filters). Purified variants were then stored at −80° C. and analyzed for concentration, purity, and in vitro activity before transfection to cells.

### RNP Electroporation of LCL Cells

An RNP mixture was prepared by mixing 124 pmol of sgRNA and 105 pmol of nuclease. LCL cells were centrifuged at room temperature, at 300×g for 5 minutes and washed with PBS. The pellet was re-suspended in an appropriate volume of Lonza Nucleofection SG electroporation solution and transferred to the RNP mixture. Electroporation was performed using 4D-Nucleofector device (Lonza Bioscience). Pre-warmed BLCL medium was added to the cuvette immediately after electroporation. Cells were incubated for 2-4 days (37° C., 5% CO₂).

### NGS Analysis

At 72 hours, cells were harvested and their genomic DNA content was used in a PCR reaction which amplified the corresponding putative genomic targets. Amplicons were subjected to next-generation sequencing (NGS) and the resulting sequences were then used to calculate the percentage of editing events at each target site. Short insertions or deletions (indels) around the cut site are the typical outcome of DNA repair following nuclease induced DNA cleavage. The calculation of percent editing was therefore deduced from the fraction of indel-containing sequences within each amplicon.

TABLE A
CRISPR Nucleases

| | | Amino acid | |
|---|---|---|---|
| | | sequence | DNA sequence |
| OMNI-50 | Mutations Relative to WT | (starting at | (starting at |
| Variant | OMNI-50 | second position) | second position) |
| Wild-type | None | SEQ ID NO: 62 | SEQ ID NO: 71 |
| V3795 | R478I, Y545H, Q803V, L805I | SEQ ID NO: 63 | SEQ ID NO: 72 |
| V6918 | Q803V | SEQ ID NO: 64 | SEQ ID NO: 73 |
| V6919 | Y545H | SEQ ID NO: 65 | SEQ ID NO: 74 |
| V6920 | L805I | SEQ ID NO: 66 | SEQ ID NO: 75 |
| V6864 | R478I, Y545H, Q803V | SEQ ID NO: 67 | SEQ ID NO: 76 |
| V6865 | R478I, Y545H, L805I | SEQ ID NO: 68 | SEQ ID NO: 77 |
| V6866 | R478I, Q803V, L805I | SEQ ID NO: 69 | SEQ ID NO: 78 |
| V6867 | Y545H, Q803V, L805I | SEQ ID NO: 70 | SEQ ID NO: 79 |
| V7085 | R478H, Y545H, Q803V, L805I | SEQ ID NO: 89 | SEQ ID NO: 98 |
| V7086 | R478V, Y545H, Q803V, L805I | SEQ ID NO: 90 | SEQ ID NO: 99 |
| V7087 | R478D, Y545H, Q803V, L805I | SEQ ID NO: 91 | SEQ ID NO: 100 |
| V7088 | R478S, Y545H, Q803V, L805I | SEQ ID NO: 92 | SEQ ID NO: 101 |
| V7090 | R478I, Y545C, Q803V, L805I | SEQ ID NO: 93 | SEQ ID NO: 102 |
| V7091 | R478I, Y545D, Q803V, L805I | SEQ ID NO: 94 | SEQ ID NO: 103 |
| V7092 | R478I, Y545T, Q803V, L805I | SEQ ID NO: 95 | SEQ ID NO: 104 |
| V7093 | R478I, Y545F, Q803V, L805I | SEQ ID NO: 96 | SEQ ID NO: 105 |
| V7094 | R478I, Y545A, Q803V, L805I | SEQ ID NO: 97 | SEQ ID NO: 106 |

TABLE B
sg62 Guide Molecule

| sgRNA Name | sgRNA Sequence (Spacer and Scaffold) |
|---|---|
| sg62 | GUGUCAAGCCCCAGAGGCCACAGUUUGAGA |
| | GUUAUGAAAAUGACGAGUUCAAAUAAAAAU |
| | UUAUUCAAACCGCCUAUUUAUAGGCCGCAG |
| | AUGUUCUGCUUU (SEQ ID NO: 80) |

TABLE C
Guide Molecule Target Sequences

| Target | DNA sequence | PAM |
|---|---|---|
| g62 | GTGTCAAGCCCCAGAGGCCACA | GGGACA |
| | (SEQ ID NO: 81) | |
| g62OT1 | GCCAAACCCCAAAGGCCACA | CGGTGT |
| | (SEQ ID NO: 82) | |
| g62OT2 | GCCAAGCCTCAGAGGCCACA | GGGCAG |
| | (SEQ ID NO: 83) | |
| g62Alt | GTGTCAAGCCCCAGAGGACACA | GGGACA |
| | (SEQ ID NO: 84) | |

TABLE D
pET9a Plasmid Utilized

| Plasmid | Purpose | Elements |
|---|---|---|
| pET9a | Expressing OMNI | T7 promoter - SV40 NLS - Linker - |
| | polypeptide in the | OMNI ORF (Human optimized) - HA Tag - |
| | bacterial system | SV40 NLS - 8XHisTag - T7 terminator |

TABLE D
Appendix: pET9a Elements

| Element | Protein Sequence | DNA sequence |
|---|---|---|
| HA Tag | YPYDVPDYA | TACCCATACGATGT |
| | (SEQ ID NO: 85) | TCCAGATTACGCT |
| | | (SEQ ID NO: 86) |
| SV40 NLS | PKKKRKV | CCAAAAAAGAAAAG |
| | (SEQ ID NO: 87) | AAAAGTT (SEQ ID |
| | | NO: 88) |

TABLE E

| | | | | | |
|---|---|---|---|---|---|
| Sample | % Unedited | | % Unedited | | Graphical |
| Name | ALT | SD | REF | SD | Representation |

Activity in LCL Cells as displayed in FIG. 17A

| | | | | | |
|---|---|---|---|---|---|
| NT | 49.94 | 0.57 | 49.92 | 0.54 | FIG. 17A |
| WT | 31.48 | 0.87 | 5.87 | 2.98 | FIG. 17A |
| V3795 | 55.31 | 0.27 | 5.52 | 0.81 | FIG. 17A |
| V6918 | 24.21 | 0.67 | 7.86 | 3.64 | FIG. 17A |
| V6919 | 24.08 | 6.60 | 4.98 | 1.33 | FIG. 17A |
| V6920 | 27.60 | 5.28 | 5.43 | 1.39 | FIG. 17A |
| V6864 | 57.41 | 0.30 | 10.34 | 1.08 | FIG. 17A |
| V6865 | 58.18 | 1.95 | 8.71 | 1.61 | FIG. 17A |
| V6866 | 60.44 | 0.76 | 7.30 | 2.38 | FIG. 17A |
| V6867 | 30.83 | 1.97 | 4.68 | 0.28 | FIG. 17A |

Activity in LCL Cells as displayed in FIG. 17B

| | | | | | |
|---|---|---|---|---|---|
| NT | 49.91 | 1.43 | 49.90 | 1.40 | FIG. 17B |
| V3795 | 62.19 | 2.45 | 6.40 | 4.32 | FIG. 17B |
| V7085 | 61.51 | 2.16 | 7.83 | 4.48 | FIG. 17B |
| V7086 | 61.08 | 2.06 | 7.04 | 3.03 | FIG. 17B |
| V7087 | 27.70 | 2.00 | 6.68 | 0.34 | FIG. 17B |
| V7088 | 56.73 | 0.04 | 8.23 | 2.71 | FIG. 17B |
| V7090 | 34.38 | 2.13 | 6.72 | 2.33 | FIG. 17B |
| V7091 | 21.23 | 2.37 | 6.59 | 0.72 | FIG. 17B |
| V7092 | 36.66 | 0.91 | 10.43 | 1.73 | FIG. 17B |
| V7093 | 59.74 | 2.75 | 10.37 | 6.44 | FIG. 17B |
| V7094 | 61.44 | 1.07 | 7.04 | 4.59 | FIG. 17B |

TABLE F
Off-target Editing as displayed in FIG. 18A

| Sample | % Editing | | % Editing | | Graphical |
|---|---|---|---|---|---|
| Name | OT1 | SD | OT2 | SD | Representation |
| NT | 0.04 | 0.01 | 0.03 | 0.01 | FIG. 18A |
| WT | 38.5 | 11.10 | 6.14 | 0.31 | FIG. 18A |
| V3795 | 0.05 | 0.01 | 0.02 | 0.00 | FIG. 18A |
| V6918 | 20.4 | 1.30 | 2.52 | 0.49 | FIG. 18A |
| V6919 | 0.5 | 0.05 | 0.04 | 0.02 | FIG. 18A |
| V6920 | 31.0 | 7.39 | 4.78 | 1.56 | FIG. 18A |
| V6864 | 0.04 | 0.01 | 0.0 | 0.01 | FIG. 18A |
| V6865 | 0.07 | 0.03 | 0.0 | 0.01 | FIG. 18A |
| V6866 | 1.90 | 0.11 | 0.2 | 0.06 | FIG. 18A |
| V6867 | 0.07 | 0.03 | 0.1 | 0.05 | FIG. 18A |

TABLE F
Off-target Editing as displayed in FIG. 18B

| Sample | % Editing | | % Editing | | Graphical |
|---|---|---|---|---|---|
| Name | OT1 | SD | OT2 | SD | Representation |
| NT | 0.03 | 0.01 | 0.02 | 0.00 | FIG. 18B |
| V3795 | 0.04 | 0.01 | 0.03 | 0.01 | FIG. 18B |
| V7085 | 0.18 | 0.21 | 0.04 | 0.02 | FIG. 18B |
| V7086 | 0.13 | 0.00 | 0.03 | 0.00 | FIG. 18B |
| V7087 | 15.92 | 0.00 | 2.56 | 0.10 | FIG. 18B |
| V7088 | 0.12 | 0.08 | 0.04 | 0.03 | FIG. 18B |
| V7090 | 7.49 | 1.27 | 0.88 | 0.14 | FIG. 18B |
| V7091 | 68.40 | 3.49 | 0.30 | 0.18 | FIG. 18B |
| V7092 | 6.87 | 1.53 | 1.05 | 0.33 | FIG. 18B |
| V7093 | 0.05 | 0.01 | 0.02 | 0.00 | FIG. 18B |
| V7094 | 0.10 | 0.00 | 0.03 | 0.01 | FIG. 18B |

### REFERENCES

1. Ahmad and Allen (1992) “Antibody-mediated Specific Binging and Cytotoxicity of Liposome-entrapped Doxorubicin to Lung Cancer Cells in Vitro”, Cancer Research 52:4817-20.
2. Anderson (1992) “Human gene therapy”, Science 256:808-13.
3. Atosuo J T, Lilius E-M. The Real-Time-Based Assessment of the Microbial Killing by the Antimicrobial Compounds of Neutrophils. Scientific World Journal. 2011; 11:2382-2390.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) “Gene transfer with synthetic cationic amphiphiles: Prospects for gene therapy”, Bioconjugate Chem 5:382-89.
6. Blaese et al. (1995) “Vectors in cancer therapy: how will they deliver”, Cancer Gene Ther. 2:291-97.
7. Blaese et al. (1995) “T lymphocyte-directed gene therapy for ADA-SCID: initial trial results after 4 years”, Science 270(5235):475-80.
8. Briner et al. (2014) “Guide RNA functional modules direct Cas9 activity and orthogonality”, Molecular Cell 56:333-39.
9. Buchschacher and Panganiban (1992) “Human immunodeficiency virus vectors for inducible expression of foreign genes”, J. Virol. 66:2731-39.
10. Burstein et al. (2017) “New CRISPR-Cas systems from uncultivated microbes”, Nature 542:237-41.
11. Canver et al., (2015) “BCL11A enhancer dissection by Cas9-mediated in situ saturating mutagenesis”, Nature Vol. 527, Pgs. 192-214.
12. Chang and Wilson (1987) “Modification of DNA ends can decrease end-joining relative to homologous recombination in mammalian cells”, Proc. Natl. Acad. Sci. USA 84:4959-4963.
13. Charlesworth et al. (2019) “Identification of preexisting adaptive immunity to Cas9 proteins in humans”, Nature Medicine, 25(2), 249.
14. Chung et al. (2006) “Agrobacterium is not alone: gene transfer to plants by viruses and other bacteria”, Trends Plant Sci. 11(1):1-4.
15. Coelho et al. (2013) “Safety and efficacy of RNAi therapy for transthyretin amyloidosis” N. Engl. J. Med. 369, 819-829.
16. Crystal (1995) “Transfer of genes to humans: early lessons and obstacles to success”, Science 270(5235):404-10.
17. Dillon (1993) “Regulation gene expression in gene therapy” Trends in Biotechnology 11(5):167-173.
18. Dranoff et al. (1997) “A phase I study of vaccination with autologous, irradiated melanoma cells engineered to secrete human granulocyte macrophage colony stimulating factor”, Hum. Gene Ther. 8(1):111-23.
19. Dunbar et al. (1995) “Retrovirally marked CD34-enriched peripheral blood and bone marrow cells contribute to long-term engraftment after autologous transplantation”, Blood 85:3048-57.
20. Ellem et al. (1997) “A case report: immune responses and clinical course of the first human use of granulocyte/macrophage-colony-stimulating-factor-transduced autologous melanoma cells for immunotherapy”, Cancer Immunol Immunother 44:10-20.
21. Gao and Huang (1995) “Cationic liposome-mediated gene transfer” Gene Ther. 2(10):710-22.
22. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
23. Han et al. (1995) “Ligand-directed retro-viral targeting of human breast cancer cells”, Proc. Natl. Acad. Sci. USA 92(21):9747-51.
24. Humbert et al., (2019) “Therapeutically relevant engraftment of a CRISPR-Cas9-edited HSC-enriched population with HbF reactivation in nonhuman primates”, Sci. Trans. Med., Vol. 11, Pgs. 1-13.
25. Inaba et al. (1992) “Generation of large numbers of dendritic cells from mouse bone marrow cultures supplemented with granulocyte/macrophage colony-stimulating factor”, J Exp Med. 176(6):1693-702.
26. Jinek et al. (2012) “A programmable dual-RNA-guided DNA endonuclease in adaptive bacterial immunity”, Science 337(6096):816-21.
27. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
28. Judge et al. (2006) “Design of noninflammatory synthetic siRNA mediating potent gene silencing in vivo”, Mol Ther. 13(3):494-505.
29. Kohn et al. (1995) “Engraftment of gene-modified umbilical cord blood cells in neonates with adenosine deaminase deficiency”, Nature Medicine 1:1017-23.
30. Kremer and Perricaudet (1995) “Adenovirus and adeno-associated virus mediated gene transfer”, Br. Med. Bull. 51(1):31-44.
31. Macdiarmid et al. (2009) “Sequential treatment of drug-resistant tumors with targeted minicells containing siRNA or a cytotoxic drug”, Nat Biotechnol. 27(7):643-51.
32. Makaryan V. et al. The diversity of mutations and clinical outcomes for ELANE-associated neutropenia. Curr. Opin. Hematol. 2015; 22(1):3-11.
33. Malech et al. (1997) “Prolonged production of NADPH oxidase-corrected granulocytes after gene therapy of chronic granulomatous disease”, PNAS 94(22):12133-38.
34. Maxwell et al. (2018) “A detailed cell-free transcription-translation-based assay to decipher CRISPR protospacer adjacent motifs”, Methods 14348-57
35. Miller et al. (1991) “Construction and properties of retrovirus packaging cells based on gibbon ape leukemia virus”, J Virol. 65(5):2220-24.
36. Miller (1992) “Human gene therapy comes of age”, Nature 357:455-60.
37. Mitani and Caskey (1993) “Delivering therapeutic genes—matching approach and application”, Trends in Biotechnology 11(5):162-66.
38. Nabel and Felgner (1993) “Direct gene transfer for immunotherapy and immunization”, Trends in Biotechnology 11(5):211-15.
39. Nasri M. et al. CRISPR/Cas9-mediated ELANE knockout enables neutrophilic maturation of primary hematopoietic stem and progenitor cells and induced pluripotent stem cells of severe congenital neutropenia patients. Haematologica. 2020; 105(3):598-609.
40. Nehls et al. (1996) “Two genetically separable steps in the differentiation of thymic epithelium” Science 272:886-889.
41. Remy et al. (1994) “Gene Transfer with a Series of Lipohilic DNA-Binding Molecules”, Bioconjugate Chem. 5(6):647-54.
42. Sentmanat et al. (2018) “A Survey of Validation Strategies for CRISPR-Cas9 Editing”, Scientific Reports 8:888, doi:10.1038/s41598-018-19441-8.
43. Sommerfelt et al. (1990) “Localization of the receptor gene for type D simian retroviruses on human chromosome 19”, J. Virol. 64(12):6214-20.
44. Van Brunt (1988) “Molecular framing: transgenic animals as bioactors” Biotechnology 6:1149-54.
45. Vigne et al. (1995) “Third-generation adenovectors for gene therapy”, Restorative Neurology and Neuroscience 8(1,2): 35-36.
46. Wagner et al. (2019) “High prevalence of Streptococcus pyogenes Cas9-reactive T cells within the adult human population” Nature Medicine, 25(2), 242
47. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
48. Yu et al. (1994) “Progress towards gene therapy for HIV infection”, Gene Ther. 1(1):13-26.
49. Zetsche et al. (2015) “Cpf1 is a single RNA-guided endonuclease of a class 2 CRIPSR-Cas system” Cell 163(3):759-71.
50. Zuris et al. (2015) “Cationic lipid-mediated delivery of proteins enables efficient protein based genome editing in vitro and in vivo” Nat Biotechnol. 33(1):73-80.

1. A non-naturally occurring OMNI-50 nuclease variant having a wild-type OMNI-50 protein sequence (SEQ ID NO: 1) comprising an amino acid substitution in at least one of the following positions: R478, Y545, R61, Y437, A493, G606, K688, L690, E695, L718, R788, Q803, L805, L844, V981, K965, and K1036.

2. The OMNI-50 nuclease variant of claim 1, comprising an amino acid substitution at position R478 and/or position Y545.

3. The OMNI-50 nuclease variant of claim 2, comprising an amino acid substitution at each of positions R478 and Y545.

4. The OMNI-50 nuclease variant of claim 2, wherein the amino acid substitution at position R478 is any one of the following substitutions: R478D, R478E, R478S, R478T, R478N, R478Q, R478G, R478P, R478C, R478A, R478V, R478I, R478L, R478M, R478F, R478Y, or R478W, preferably R478V, R478H, R478L, R478M, R478P, R478F, R478W, R478Y, R478S, R478C, R478T, R478N, or R478Q.

5. The OMNI-50 nuclease variant of claim 2, wherein the amino acid substitution is at position R478 and the amino acid substituted for arginine is an amino acid having a negatively charged R-group or an R-group lacking a charge.

6. The OMNI-50 nuclease variant of claim 2, wherein the amino acid substitution is at position R478 and the amino acid substituted for arginine is a polar amino acid or non-polar amino acid.

7. The OMNI-50 nuclease variant of claim 2, wherein the amino acid substitution is at position R478 and the amino acid substituted for arginine is a non-polar amino acid.

8. The OMNI-50 nuclease variant of claim 2, wherein the amino acid substitution at position Y545 is any one of the following substitutions: Y545D, Y545E, Y545S, Y545T, Y545N, Y545Q, Y545G, Y545P, Y545C, Y545A, Y545V, Y545I, Y545L, Y545M, Y545F, Y545R, Y545K, Y545H, or Y545W.

9. The OMNI-50 nuclease variant of claim 2, wherein the amino acid substitution is at position Y545 and the amino acid substituted for tyrosine is an amino acid having a negatively charged R-group or a positively charged R-group.

10. The OMNI-50 nuclease variant of claim 2, wherein the amino acid substitution is at position Y545 and the amino acid substituted for tyrosine is a non-polar amino acid.

11. The OMNI-50 nuclease variant of claim 2, wherein the amino acid substitution is at position Y545 and the amino acid substituted for tyrosine lacks a phenyl ring or is phenylalanine.

12. The OMNI-50 nuclease variant of claim 3, wherein the amino acid substitutions are R478I and Y545H.

13. The OMNI-50 nuclease variant of claim 1, wherein the amino acid substitution is any one of the following substitutions: R478I, Y545H, Q803V, L805I, R61A, Y437W, R788K, L844N, V981M, G606P, L690V, E695Q, R478T, A493G, K688E, L718C, K965V, and K1036V.

14. The OMNI-50 nuclease variant of claim 1, comprising an amino acid substitution at each of positions R478, Y545, Q803, and L805.

15. The OMNI-50 nuclease variant of claim 14, wherein the amino acid substitutions are R478I, Y545H, Q803V, and L805I.

16. The OMNI-50 nuclease variant of claim 1, comprising an amino acid substitution at each of positions R61, Y437, R788, L844, and V981.

17. The OMNI-50 nuclease variant claim 16, wherein the amino acid substitutions are R61A, Y437W, R788K, L844N, and V981M.

18. The OMNI-50 nuclease variant of claim 1, comprising an amino acid substitution at each of positions G606, L690, and E695.

19. The OMNI-50 nuclease variant of claim 18, wherein the amino acid substitutions are G606P, L690V, and E695Q.

20. The OMNI-50 nuclease variant of claim 1, comprising an amino acid substitution at each of positions R478, A493, K688, L718, K965, and K1036.

21. The OMNI-50 nuclease variant of claim 20, wherein the amino acid substitutions are R478T, A493G, K688E, L718C, K965V, and K1036V.

22. The OMNI-50 nuclease variant of claim 1, comprising an amino acid substitution at position Q803.

23. The OMNI-50 nuclease variant of claim 22, wherein the amino acid substitution is Q803V.

24. The OMNI-50 nuclease variant of claim 1, comprising an amino acid substitution at position Y545.

25. The OMNI-50 nuclease variant of claim 24, wherein the amino acid substitution is Y545H.

26. The OMNI-50 nuclease variant of claim 1, comprising an amino acid substitution at position L805.

27. The OMNI-50 nuclease variant of claim 26, wherein the amino acid substitution is L805I.

28. The OMNI-50 nuclease variant of claim 1 comprising an amino acid substitution at each of positions R478, Y545, and Q803.

29. The OMNI-50 nuclease variant of claim 28, comprising an amino acid substitutions are R478I, Y545H, AND Q803V.

30. The OMNI-50 nuclease variant of claim 1, comprising an amino acid substitution at each of positions R478, Q803, and L805.

31. The OMNI-50 nuclease variant of claim 30, wherein the amino acid substitutions are R478I, Q803V, and L805I.

32. The OMNI-50 nuclease variant of claim 1, comprising an amino acid substitution at each of positions R478, Q803, and L805.

33. The OMNI-50 nuclease variant of claim 32, wherein the amino acid substitutions are R478I, Q803V, and L805I.

34. The OMNI-50 nuclease variant of claim 1, comprising an amino acid substitution at each of positions Y545, Q803, and L805.

35. The OMNI-50 nuclease variant claim 34, wherein the amino acid substitutions are R478I, Y545H, and L805I.

36. The OMNI-50 nuclease variant of claim 1, having an amino acid sequence selected from the group consisting of SEQ ID NOs: 2-5, 63-70, and 89-97.

37. The OMNI-50 nuclease variant of claim 1, having at least 80% sequence identity to the wild-type OMNI-50 protein sequence (SEQ ID NO:1).

38. The OMNI-50 nuclease variant of claim 1, further comprising a nuclear localization sequence (NLS).

39. The OMNI-50 nuclease variant of claim 1, wherein the variant exhibits increased specificity toward a DNA target site when complexed with a guide RNA molecule that targets variant to the said DNA target site relative to a wild-type OMNI-50 nuclease complexed with the guide RNA molecule.

40. A CRISPR system comprising the OMNI-50 nuclease variant of claim 1, wherein the OMNI-50 nuclease variant is complexed with a guide RNA molecule that targets a DNA target site, and wherein the CRISPR system displays reduced off-target editing activity relative to a wild-type CRISPR system comprising a wild-type OMNI-50 nuclease protein and the guide RNA molecule.

41. A method for gene editing having reduced off-target editing activity, comprising contacting a DNA target site with an active CRISPR system comprising an OMNI-50 nuclease variant protein of claim 1.

42. The method of claim 41, wherein the active CRISPR system displays reduced off-target editing activity relative to a wild-type CRISPR system comprising a wild-type OMNI-50 nuclease protein.

43. The method of claim 41, wherein the gene editing occurs in a eukaryotic cell or prokaryotic cell.

44. The method of claim 43, wherein the eukaryotic cell is a plant cell or mammalian cell.

45. The method of claim 44, wherein the mammalian cell is a human cell.

46. The method of claim 41, wherein the DNA target site is located within or in proximity to a pathogenic allele of a gene.

47. The method of claim 46, wherein the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, RPE65, LDLR, ANGPTL3, B2M, TRAC, TCF4, TGFBi, PAX6, C3, LRRK2, SARM1, SAMD9, SAMD9L, HAVCR2, CD3E, APLP2, CISH, TIGIT, TNNT2, TNN, MYH7, and HLA-E.

48. The method of claim 41 wherein the DNA target is repaired with an exogenous donor molecule.

49. The method of claim 41, wherein the off-target editing activity is reduced by at least 2-fold, 10-fold, 10²-fold, 10³-fold, 10⁴-fold, 10⁵-fold, or 10⁶-fold.

50. A modified cell obtained by the method of claim 43.

51. The modified cell of claim 50, wherein the cell is capable of engraftment.

52. The modified cell of claim 51, wherein the cell is capable of giving rise to progeny cells after engraftment.

53. The modified cell of claim 51, wherein the cell is capable of giving rise to progeny cells after an autologous engraftment.

54. The modified cell of claim 51, wherein the cell is capable of giving rise to progeny cells for at least 12 months or at least 24 months after engraftment.

55. The modified cell of claim 51, wherein the cell is selected from the group consisting of a hematopoietic stem cell, a progenitor cell, a CD34+ hematopoietic stem cell, a bone marrow cell, and a peripheral mononucleated cell.

56. A composition comprising a modified cell of claim 50 and a pharmaceutically acceptable carrier.

57. An in vitro or ex vivo method of preparing the composition of claim 56, comprising mixing the cells with the pharmaceutically acceptable carrier.

58. A polynucleotide molecule encoding the OMNI-50 variant protein claim 1.


SEQUENCE LISTING (106 sequences)

SEQ ID NO: 1 - protein, 1370 aa, unidentified
  REGION 1..1370: note = Wild-type OMNI-50 nuclease
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 2 - protein, 1370 aa, synthetic construct
  REGION 1..1370: note = Variant 3795
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 3 - protein, 1370 aa, synthetic construct
  REGION 1..1370: note = Variant 3942
MTKVKDYYIGLDIGTSSVGWAVTDEAYNVLKFNSKKMWGVRLFDDAKTAEERRGQRGARRALDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPWQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMAKGKEEVKARKESRKNQLLKFYKDGKKAFISEIGEERYSYLLSEIEGEEENKFRWDNNYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIMYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 4 - protein, 1370 aa, synthetic construct
  REGION 1..1370: note = Variant 2902
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSPTDGDFKQGLNSYNDFKAIVGDKVDSDDYRDKIEEIIKLIVLYGDDKSYLQKKIKAGYGKYFTDSEIKKMAGLNYKDWGRLSKKVLTGLQGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 5 - protein, 1370 aa, synthetic construct
  REGION 1..1370: note = Variant 3954
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFTIPYYVGPLNPNSKYGWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSEKLLTGLEGANKITGERGSIIHFMREYNLNCMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATVETANLLKTICKNSEIVYSKAENASRFRQEFDIVKCRAVNDLHHMHDAYINIIVGNVYNTKFTKDPMNFVKVQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 6 - DNA, 4110 bp, unidentified
  misc_feature 1..4110: note = Wild-type OMNI-50 nuclease DNA sequence
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 7 - DNA, 4110 bp, synthetic construct
  misc_feature 1..4110: note = Variant 3795 DNA sequence
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 8 - DNA, 4110 bp, synthetic construct
  misc_feature 1..4110: note = Variant 3942 DNA sequence
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaaaaccgcggaagagcgccgtggccagcgcggcgcgcgccgtgcgctggatcgcaaaaaggaacgcctcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaaaagctcggataacggcgtgattccgtggcagctgaaactgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctaagggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacaactacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcatgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 9 - DNA, 4110 bp, synthetic construct
  misc_feature 1..4110: note = Variant 2902 DNA sequence
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctccgaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaaggttctgacgggactccagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 10 - DNA, 4110 bp, synthetic construct
  misc_feature 1..4110: note = Variant 3954 DNA sequence
  source 1..4110: mol_type = other DNA
atgaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctcaatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaaaagctcggataacggcgtgattccgtatcagctgaaactgatggaattgaacaaaatactcgagaatcagtcaaagcatcacgagttcctcaatgtcagcgacgagtacggctccgtgtgtgataaaatcgcatctatcatggaatttaccattccgtattacgtgggcccgctgaacccgaacagcaaatatggctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcagaaaagctgctgacgggactcgagggtgcaaacaagatcactggagagcggggctccattattcacttcatgagggaatataaccttaattgtatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacagtcgagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaaggtccaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 11 - protein, 1410 aa, synthetic construct
  REGION 1..1410: note = Wild-type OMNI-50 nuclease with tags
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 12 - protein, 1410 aa, synthetic construct
  REGION 1..1410: note = Variant 3795 with tags
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 13 - protein, 1410 aa, synthetic construct
  REGION 1..1410: note = Variant 3942 with tags
MPKKKRKVGTTKVKDYYIGLDIGTSSVGWAVTDEAYNVLKFNSKKMWGVRLFDDAKTAEERRGQRGARRALDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPWQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMAKGKEEVKARKESRKNQLLKFYKDGKKAFISEIGEERYSYLLSEIEGEEENKFRWDNNYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIMYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 14 - protein, 1410 aa, synthetic construct
  REGION 1..1410: note = Variant 2902 with tags
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSPTDGDFKQGLNSYNDFKAIVGDKVDSDDYRDKIEEIIKLIVLYGDDKSYLQKKIKAGYGKYFTDSEIKKMAGLNYKDWGRLSKKVLTGLQGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 15 - protein, 1410 aa, synthetic construct
  REGION 1..1410: note = Variant 3954 with tags
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFTIPYYVGPLNPNSKYGWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSEKLLTGLEGANKITGERGSIIHFMREYNLNCMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATVETANLLKTICKNSEIVYSKAENASRFRQEFDIVKCRAVNDLHHMHDAYINIIVGNVYNTKFTKDPMNFVKVQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 16 - DNA, 4233 bp, synthetic construct
  misc_feature 1..4233: note = Wild-type OMNI-50 nuclease with tags DNA sequence
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 17 - DNA, 4233 bp, synthetic construct
  misc_feature 1..4233: note = Variant 3795 with tags DNA sequence
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 18 - DNA, 4233 bp, synthetic construct
  misc_feature 1..4233: note = Variant 3942 with tags DNA sequence
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaaaaccgcggaagagcgccgtggccagcgcggcgcgcgccgtgcgctggatcgcaaaaaggaacgcctcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaaaagctcggataacggcgtgattccgtggcagctgaaactgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctaagggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacaactacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcatgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 19 - DNA, 4233 bp, synthetic construct
  misc_feature 1..4233: note = Variant 2902 with tags DNA sequence
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctccgaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaaggttctgacgggactccagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 20 - DNA, 4233 bp, synthetic construct
  misc_feature 1..4233: note = Variant 3954 with tags DNA sequence
  source 1..4233: mol_type = other DNA
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctcaatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaaaagctcggataacggcgtgattccgtatcagctgaaactgatggaattgaacaaaatactcgagaatcagtcaaagcatcacgagttcctcaatgtcagcgacgagtacggctccgtgtgtgataaaatcgcatctatcatggaatttaccattccgtattacgtgggcccgctgaacccgaacagcaaatatggctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcagaaaagctgctgacgggactcgagggtgcaaacaagatcactggagagcggggctccattattcacttcatgagggaatataaccttaattgtatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacagtcgagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaaggtccaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaa

SEQ ID NO: 21 - protein, 7 aa, synthetic construct
  REGION 1..7: note = Nt-NLS
PKKKRKV

SEQ ID NO: 22 - protein, 9 aa, synthetic construct
  REGION 1..9: note = HA tag
YPYDVPDYA

SEQ ID NO: 23 - protein, 7 aa, synthetic construct
  REGION 1..7: note = Ct-NLS
PKKKRKV

SEQ ID NO: 24 - protein, 8 aa, synthetic construct
  REGION 1..8: note = X8 His-tag
HHHHHHHH

SEQ ID NO: 25 - DNA, 21 bp, synthetic construct
  misc_feature 1..21: note = Nt-NLS DNA sequence
  source 1..21: mol_type = other DNA
cctaagaagaagagaaaggtg

SEQ ID NO: 26 - DNA, 27 bp, synthetic construct
  misc_feature 1..27: note = HA tag DNA sequence
  source 1..27: mol_type = other DNA
tacccatacgatgttccagattacgcg

SEQ ID NO: 27 - DNA, 21 bp, synthetic construct
  misc_feature 1..21: note = Ct-NLS DNA sequence
  source 1..21: mol_type = other DNA
ccaaaaaagaaaagaaaagtt

SEQ ID NO: 28 - DNA, 24 bp, synthetic construct
  misc_feature 1..24: note = X8 His-tag DNA sequence
  source 1..24: mol_type = other DNA
catcatcaccatcaccatcatcat

SEQ ID NO: 29 - RNA, 20 nt, synthetic construct
  misc_feature 1..20: note = g62 REF 20 nt
  source 1..20: mol_type = other RNA
gtcaagccccagaggccaca

SEQ ID NO: 30 - RNA, 20 nt, synthetic construct
  misc_feature 1..20: note = g62 ALT 20 nt
  source 1..20: mol_type = other RNA
gtcaagccccagaggacaca

SEQ ID NO: 31 - RNA, 22 nt, synthetic construct
  misc_feature 1..22: note = sgRNA-564DS-ref 22 nt
  source 1..22: mol_type = other RNA
gtgtcaagccccagaggccaca

SEQ ID NO: 32 - RNA, 22 nt, synthetic construct
  misc_feature 1..22: note = sgRNA-564DS-alt 22 nt
  source 1..22: mol_type = other RNA
gtgtcaagccccagaggacaca

SEQ ID NO: 33 - RNA, 20 nt, synthetic construct
  misc_feature 1..20: note = g35 20 nt
  source 1..20: mol_type = other RNA
agtccgggctgggagcgggt

SEQ ID NO: 34 - RNA, 22 nt, synthetic construct
  misc_feature 1..22: note = sgRNA-Constant 22 nt
  source 1..22: mol_type = other RNA
gcagtccgggctgggagcgggt

SEQ ID NO: 35 - RNA, 102 nt, synthetic construct
  misc_feature 1..102: note = Synthetic sequence
  source 1..102: mol_type = other RNA
gtgtcaagccccagaggccacagtttgagagttatgaaaatgacgagttcaaataaaaatttattcaaaccgcctatttataggccgcagatgttctgcttt

SEQ ID NO: 36 - RNA, 102 nt, synthetic construct
  misc_feature 1..102: note = Synthetic sequence
  source 1..102: mol_type = other RNA
gtgtcaagccccagaggacacagtttgagagttatgaaaatgacgagttcaaataaaaatttattcaaaccgcctatttataggccgcagatgttctgcttt

SEQ ID NO: 37 - RNA, 102 nt, synthetic construct
  misc_feature 1..102: note = Synthetic sequence
  source 1..102: mol_type = other RNA
gcagtccgggctgggagcgggtgtttgagagttatgaaaatgacgagttcaaataaaaatttattcaaaccgcctatttataggccgcagatgttctgcttt

SEQ ID NO: 38 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnntacgaccccgtaaacttgct

SEQ ID NO: 39 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnncggagcgttggatgatagag

SEQ ID NO: 40 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnntcgcagtccagcttccccac

SEQ ID NO: 41 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnnacagccaaggagcatcaaac

SEQ ID NO: 42 - DNA, 20 bp, synthetic construct
  misc_feature 1..20: note = Synthetic sequence
  source 1..20: mol_type = other DNA
acgtctgcactctcgtgagg

SEQ ID NO: 43 - DNA, 22 bp, synthetic construct
  misc_feature 1..22: note = Synthetic sequence
  source 1..22: mol_type = other DNA
ttcaggctccacccagtttgtc

SEQ ID NO: 44 - DNA, 21 bp, synthetic construct
  misc_feature 1..21: note = Synthetic sequence
  source 1..21: mol_type = other DNA
gcacagggctataagaggagc

SEQ ID NO: 45 - DNA, 20 bp, synthetic construct
  misc_feature 1..20: note = Synthetic sequence
  source 1..20: mol_type = other DNA
gcgggaggttggactcaaaa

SEQ ID NO: 46 - DNA, 19 bp, synthetic construct
  misc_feature 1..19: note = Synthetic sequence
  source 1..19: mol_type = other DNA
gggagaggaagtggagggc

SEQ ID NO: 47 - DNA, 18 bp, synthetic construct
  misc_feature 1..18: note = Synthetic sequence
  source 1..18: mol_type = other DNA
gagggtcatggtggggct

SEQ ID NO: 48 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnnggtgggtcctcagtgactct

SEQ ID NO: 49 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnngggaattccagcctgaccaa

SEQ ID NO: 50 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnngagtgaggaccaagcctgag

SEQ ID NO: 51 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnnagggccattgtctccctaac

SEQ ID NO: 52 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnntcctgctaccctcccttctt

SEQ ID NO: 53 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnnttttaggaggggcccactga

SEQ ID NO: 54 - DNA, 20 bp, synthetic construct
  misc_feature 1..20: note = Synthetic sequence
  source 1..20: mol_type = other DNA
gagggtcatcatcactgccc

SEQ ID NO: 55 - DNA, 27 bp, synthetic construct
  misc_feature 1..27: note = Synthetic sequence
  source 1..27: mol_type = other DNA
gccagactcacaccagagtcgacaagt

SEQ ID NO: 56 - DNA, 20 bp, synthetic construct
  misc_feature 1..20: note = Synthetic sequence
  source 1..20: mol_type = other DNA
taatacgactcactataggg

SEQ ID NO: 57 - DNA, 18 bp, synthetic construct
  misc_feature 1..18: note = Synthetic sequence
  source 1..18: mol_type = other DNA
atttaggtgacactatag

SEQ ID NO: 58 - DNA, 56 bp, synthetic construct; identical to SEQ ID NO: 40
  variation 1..56: note = Synthetic sequence
  variation 34..36: note = n is a, c, g, or t
  source 1..56: mol_type = other DNA

SEQ ID NO: 59 - DNA, 57 bp, synthetic construct; identical to SEQ ID NO: 41
  variation 1..57: note = Synthetic sequence
  variation 35..37: note = n is a, c, g, or t
  source 1..57: mol_type = other DNA

SEQ ID NO: 60 - DNA, 56 bp, synthetic construct
  variation 1..56: note = Synthetic sequence
  variation 34..36: note = n is a, c, g, or t
  source 1..56: mol_type = other DNA
tcgtcggcagcgtcagatgtgtataagagacagnnngagggtcatcatcactgccc

SEQ ID NO: 61 - DNA, 57 bp, synthetic construct
  variation 1..57: note = Synthetic sequence
  variation 35..37: note = n is a, c, g, or t
  source 1..57: mol_type = other DNA
gtctcgtgggctcggagatgtgtataagagacagnnnagtccgggctgggagcgggt

SEQ ID NO: 62 - protein, 1369 aa, synthetic construct
  REGION 1..1369: note = Wild-type OMNI-50
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 63 - protein, 1369 aa, synthetic construct
  REGION 1..1369: note = V3795
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 64 - protein, 1369 aa, synthetic construct
  REGION 1..1369: note = V6918
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 65 - protein, 1369 aa, synthetic construct
  REGION 1..1369: note = V6919
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 66 - protein, 1369 aa, synthetic construct
  REGION 1..1369: note = V6920
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 67 - protein, 1369 aa, synthetic construct
  REGION 1..1369: note = V6864
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 68 - protein, 1369 aa, synthetic construct
  REGION 1..1369: note = V6865
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 69 - protein, 1369 aa, synthetic construct
  REGION 1..1369: note = V6866
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 70 - protein, 1369 aa, synthetic construct
  REGION 1..1369: note = V6867
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 71 - DNA, 4107 bp, synthetic construct
  misc_feature 1..4107: note = Wild-type OMNI-50
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 72 - DNA, 4107 bp, synthetic construct
  misc_feature 1..4107: note = V3795
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 73 - DNA, 4107 bp, synthetic construct
  misc_feature 1..4107: note = V6918
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgttctgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 74 - DNA, 4107 bp, synthetic construct
  misc_feature 1..4107: note = V6919
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 75 - DNA, 4107 bp, synthetic construct
  misc_feature 1..4107: note = V6920
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 76 - DNA, 4107 bp, synthetic construct
  misc_feature 1..4107: note = V6920
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 77 - DNA, 4107 bp, synthetic construct
  misc_feature 1..4107: note = V6865
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 78 - DNA, 4107 bp, synthetic construct
  misc_feature 1..4107: note = V6866
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 79 - DNA, 4107 bp, synthetic construct
  misc_feature 1..4107: note = V6867
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttcgtattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 80 - RNA, 102 nt, synthetic construct; identical to SEQ ID NO: 35
  misc_feature 1..102: note = sg62 spacer and scaffold sequence
  source 1..102: mol_type = other RNA

SEQ ID NO: 81 - DNA, 22 bp, synthetic construct
  misc_feature 1..22: note = g62 target
  source 1..22: mol_type = other DNA
gtgtcaagccccagaggccaca

SEQ ID NO: 82 - DNA, 20 bp, synthetic construct
  misc_feature 1..20: note = g62OT1
  source 1..20: mol_type = other DNA
gccaaaccccaaaggccaca

SEQ ID NO: 83 - DNA, 20 bp, synthetic construct
  misc_feature 1..20: note = g62OT2
  source 1..20: mol_type = other DNA
gccaagcctcagaggccaca

SEQ ID NO: 84 - DNA, 22 bp, synthetic construct
  misc_feature 1..22: note = g62Alt target
  source 1..22: mol_type = other DNA
gtgtcaagccccagaggacaca

SEQ ID NO: 85 - protein, 9 aa, synthetic construct
  REGION 1..9: note = HA Tag
YPYDVPDYA

SEQ ID NO: 86 - DNA, 27 bp, synthetic construct
  misc_feature 1..27: note = HA Tag
  source 1..27: mol_type = other DNA
tacccatacgatgttccagattacgct

SEQ ID NO: 87 - protein, 7 aa, synthetic construct
  REGION 1..7: note = SV40 NLS
PKKKRKV

SEQ ID NO: 88 - DNA, 21 bp, synthetic construct
  misc_feature 1..21: note = SV40 NLS
  source 1..21: mol_type = other DNA
ccaaaaaagaaaagaaaagtt

SEQ ID NO: 89 - protein, 1369 aa, synthetic construct
  REGION 1..1369: note = V7085 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFHIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 90 - protein, 1369 aa, synthetic construct
  REGION 1..1369: note = V7086 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFVIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 91 - protein, 1369 aa, synthetic construct
  REGION 1..1369: note = V7087 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFDIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 92 - protein, 1369 aa, synthetic construct
  REGION 1..1369: note = V7088 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFSIPYYVGPLNPNSKYAWIKKQKDSEITPWNFKDVVDLDSSREEFIDSLIGRCTYLKDEKVLPKASLLHNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 93 - protein, 1369 aa, synthetic construct
  REGION 1..1369: note = V7090 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLCNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 94 - protein, 1369 aa, synthetic construct
  REGION 1..1369: note = V7091 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLDNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 95 - protein, 1369 aa, synthetic construct
  REGION 1..1369: note = V7092 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLTNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 96 - protein, 1369 aa, synthetic construct
  REGION 1..1369: note = V7093 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLFNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 97 - protein, 1369 aa, synthetic construct
  REGION 1..1369: note = V7094 Variant
TKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFIIPYYVGPLNPNSKYAWIKKQKDSEITPWNFKDVVDLDSSREEFIDSLIGRCTYLKDEKVLPKASLLANEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNVLIKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 98 - DNA, 4107 bp, synthetic construct
  misc_feature 1..4107: note = V7085 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttcatattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 99 - DNA, 4107 bp, synthetic construct
  misc_feature 1..4107: note = V7086 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttgtgattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 100 - DNA, 4107 bp, synthetic construct
  misc_feature 1..4107: note = V7087 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttgatattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 101 - DNA, 4107 bp, synthetic construct
  misc_feature 1..4107: note = V7088 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttagcattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgcataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 102 - DNA, 4107 bp, synthetic construct
  misc_feature 1..4107: note = V7090 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtgtaacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 103 - DNA, 4107 bp, synthetic construct
  misc_feature 1..4107: note = V7091 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctggataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 104 - DNA, 4107 bp, synthetic construct
  misc_feature 1..4107: note = V7092 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgactaacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 105 - DNA, 4107 bp, synthetic construct
  misc_feature 1..4107: note = V7093 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtttaacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 106 - DNA, 4107 bp, synthetic construct
  misc_feature 1..4107: note = V7094 Variant
  source 1..4107: mol_type = other DNA
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggaatttattattccgtattacgtgggcccgctgaacccgaacagcaaatatgcgtggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctggctaacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaacgtcctgatcaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg